(19) United States
(12) Patent Application Publication
(10) Pub. No.: US 2012/0010266 A1
(43) Pub. Date: Jan. 12, 2012

(54) TBC1D7 AS TUMOR MARKER AND THERAPEUTIC TARGET FOR CANCER

(75) Inventors: Yusuke Nakamura, Tokyo (JP); Yataro Daigo, Tokyo (JP); Akira Togashi, Kanagawa (JP)

(73) Assignee: Oncotherapy Science Inc.,, Kanagawa (JP)

(21) Appl. No.: 13/061,118

(22) PCT Filed: Aug. 14, 2009
(86) PCT No.: PCT/JP2009/003895

§ 371 Date: Feb. 25, 2011

Related U.S. Application Data
(60) Provisional application No. 61/190,522, filed on Aug. 28, 2008.

(51) Int. Cl. A61K 31/713; C12N 15/113; C12N 15/63; C07K 7/08; G01N 33/566; C07K 14/47; G01N 33/574; C40B 30/04; C07K 16/18; A61P 35/00; C12Q 1/68; C07H 21/00

(52) U.S. Cl. 514/44 A; 435/6.13; 536/24.5; 435/320.1; 530/326; 536/23.53; 530/324; 435/6.12; 435/6.11; 435/7.1; 506/9; 530/389.1; 536/24.31; 536/24.33

(57) ABSTRACT

The present invention relates to the roles played by the TBC1D7 genes in cancer, in particular, lung cancer or esophageal cancer, or carcinogenesis and features a method for treating and/or preventing cancer, in particular, lung cancer or esophageal cancer by administering a double-stranded molecule against one or more of the TBC1D7 genes or a composition, vector or cell containing such a double stranded molecule. The present invention also features methods for diagnosing lung or assessing/determining the prognosis of a patient with lung, especially NSCLC or SCLC, or esophageal cancer, using one or more over-expressed genes selected from among TBC1D7. To that end, TBC1D7 may serve as a novel biomarker for lung cancer or esophageal cancer. Also, disclosed are methods of identifying compounds for treating and preventing lung or esophageal cancer, using as an index for their effect on the over-expression of one or more of TBC1D7 in the lung cancer or esophageal cancer.

### PRIORITY

The present application claims the benefit of U.S. Provisional Application No. 61/190,522, filed on Aug. 28, 2008, the entire content of which is incorporated by reference herein.

### TECHNICAL FIELD

The present invention relates to lung cancer, more particularly the diagnosis and treatment thereof.

### BACKGROUND ART

Lung cancer is one of the most common cancers in the world, and non-small cell lung cancer (NSCLC) accounts for 80% of those cases (Greenlee R T et al. CA Cancer J Clin 2001; 51: 15-36 (NPL 1)). Many genetic alterations involved in lung carcinogenesis have been reported, but the precise molecular mechanisms still remain unclear (Sozzi G. Eur J Cancer 2001; 37 Suppl 7:S63-73 (NPL 2)). In the last few decades newly developed cytotoxic agents including paclitaxel, docetaxel, gemcitabine, and vinorelbine have emerged to offer multiple therapeutic choices for patients with advanced NSCLC, however, those regimens provide a limited survival benefit compared with cisplatin-based therapies (Schiller J H. et al. N Engl J Med 2002; 346:92-8 (NPL 3)). Esophageal squamous cell carcinoma (ESCC) is one of the most lethal malignancies of the digestive tract, and the overall 5-years survival rate of lung cancer is only 15% (Shimada H, et al., Surgery. 2003 May; 133(5):486-94 (NPL 4)). The highest incidence of esophageal cancer was reported in the area called “Asian esophageal cancer belt”, which extends from the eastern shores of the Caspian Sea to central China (Mosavi-Jarrahi A & Mohagheghi M A. Asian Pac J Cancer Prey. 2006 July-September; 7(3):375-80 (NPL 5)). Although many genetic alterations involved in development and/or progression of lung and esophagus cancer have been reported, the precise molecular mechanism remains unclear (Sozzi G. Eur J Cancer. 2001 October; 37 Suppl 7:S63-73 (NPL 2)).

In addition to these cytotoxic drugs, several molecular-targeted agents such as monoclonal antibodies against VEGF (i.e., bevacizumab/anti-VEGF) or EGFR (i.e., cetuximab/anti-EGFR) as well as inhibitors for EGFR tyrosine kinase (i.e., gefitinib and erlotinib) have been developed and are applied in clinical practice (Thatcher N. et al. Lancet 2005; 366:1527-37. (NPL 6), Shepherd F A. et al. N Engl J Med 2005; 353:123-32 (NPL 7)). Each of the new regimens can provide survival benefits to a limited subset of the patients. Hence, new therapeutic strategies, such as development of more effective molecular-targeted agents applicable to the great majority of patients with less toxicity, are eagerly awaited.

Genome-wide analysis of expression levels of thousands of genes using cDNA microarrays is an effective approach for identifying unknown molecules involved in pathways of carcinogenesis, which are good candidate targets for the development of new therapeutics and diagnostics (Daigo Y and Nakamura Y. Gen Thorac Cardiovasc Surg 2008; 56:43-53 (NPL 8)). The present inventers isolated a number of potential molecular targets for diagnosis and/or treatment of lung cancer by means of genome-wide expression profile analyses of 101 cases of lung cancers and 19 ESCCs whose tumor-cell populations were purified by laser microdissection on a cDNA microarray containing 27,648 genes, and their comparison with the expression profile data of 31 normal human tissues (27 adult and 4 fetal organs) (Kikuchi T. et al. Oncogene 2003; 22:2192-205. (NPL 9), Kakiuchi S. et al. Mol Cancer Res 2003; 1:485-99. (NPL 10), Kakiuchi S. et al. Hum Mol Genet 2004; 13:3029-43. (NPL 11), Kikuchi T. et al. Int J Oncol v2006; 28:799-805. (NPL 12), Taniwaki M. et al. Int J Oncol 2006; 29:567-75. (NPL 13), Yamabuki T. et al. Int J Oncol 2006; 28:1375-84 (NPL 14)). To verify the biological and clinicopathological significance of the respective gene products, the present inventors have established a screening system by a combination of the tumor-tissue microarray analysis of clinical lung-cancer materials and RNA interference (RNAi) technique (Suzuki C. et al. Cancer Res 2003; 63:7038-41. (NPL 15), Takahashi K. et al. Cancer Res 2006; 66:9408-19. (NPL 16), Mizukami Y. et al. Cancer Sci 2008; 99:1448-54. (NPL 17), Suzuki C. et al. Cancer Res 2003; 63:7038-41. (NPL 18), Ishikawa N. et al. Clin Cancer Res 2004; 10:8363-70. (NPL 19), Kato T. et al. Cancer Res 2005; 65:5638-46. (NPL 20), Furukawa C. et al. Cancer Res 2005; 65:7102-10. (NPL 21), Ishikawa N. et al. Cancer Res 2005; 65:9176-84. (NPL 22), Suzuki C. et al. Cancer Res 2005; 65:11314-25. (NPL 23), Ishikawa N. et al. Cancer Sci 2006; 97:737-45. (NPL 24), Takahashi K. et al. Cancer Res 2006; 66:9408-19. (NPL 25), Hayama S. et al. Cancer Res 2006; 66:10339-48. (NPL 26), Kato T. et al. Clin Cancer Res 2007; 13:434-42. (NPL 27), Suzuki C. et al. Mol Cancer Ther 2007; 6:542-51. (NPL 28), Yamabuki T. et al. Cancer Res 2007; 67:2517-25. (NPL 29), Hayama S. et al. Cancer Res 2007; 67:4113-22. (NPL 30), Kato T. et al. Cancer Res 2007; 67:8544-53. (NPL 31), Taniwaki M. et al. Clin Cancer Res 2007; 13:6624-31. (NPL 32), Ishikawa N. et al. Cancer Res 2007; 67:11601-11. (NPL 33), Mano Y. et al. Cancer Sci 2007; 98:1902-13. (NPL 34), Suda T. et al. Cancer Sci 2007; 98:1803-8. (NPL 35), Kato T. et al. Clin Cancer Res Res 2008; 14:2363-70. (NPL 36), Mizukami Y. et al. Cancer Sci 2008; 99:1448-54 (NPL 37)). In the course of these systematic studies, TBC1 domain family, member 7 (TBC1D7) was found to be overexpressed in the great majority of the lung cancers and ESCCs.

Human TBC1D7 consists of 293 amino acids with a putative TBC domain composed of approximately 200 amino acid residues. The TBC domain is conserved among eukaryotes, and the human genome is predicted to encode at least 50 proteins with this domain (Richardson P M and Zon L I. Oncogene 1995; 11: 1139-48. (NPL 38), Bernards A. Biochim Biophys Acta. 2003; 1603: 47-82 (NPL 39)). The TBC domain is considered to have a putative GTPase-activating role (GAP) for Ypt/Rab-like small G proteins (Bernards A. Biochim Biophys Acta. 2003; 1603: 47-82 (NPL 39), Neuwald A F. Trends Biochem Sci. 1997; 22: 243-4 (NPL 40)). GAPs enhance the inherently slow GTPase activity of G proteins, causing their inactivation and thus modulating the cellular pathways controlled by the respective G proteins. The Ypt/Rab family of GTPases contains 11 genes in Saccharomyces cerevisiae, and at least 60 human genes, being the largest branch of the Ras superfamily (Bernards A. Biochim Biophys Acta. 2003; 1603: 47-82 (NPL 39)). These proteins serve critical roles in the cellular processes that involve vesicular transport, being particularly important in vesicle-target membrane recognition, docking, and membrane fusion (Chavrier P and Goud B. Curr Opin Cell Biol. 1999; 11:466-75 (NPL 41)). In higher eukaryotes, the TBC domain is present in proteins (e.g., RN-TRE, TRE2, PRC17), which are associated with cell cycle and oncogenesis (Neuwald A F. Trends Biochem Sci. 1997; 22:243-4 (NPL 40), L. Pei. et al. Cancer Res. 2002; 62:5420-24 (NPL 42)). TBC1D7 acts on Rab17 as a cognate GTPase-activating proteins (GAPs) in primary cilia formation (Yoshimura S. et al. J Cell Biol. 2007; 178: 363-9 (NPL 43)). Rab17 has been previously reported to be induced during cell polarization and to be involved in the function of apical sorting endosomes in polarized epithelial cells (Lutcke A. et al. J Cell Biol. 1993; 121:553-64. (NPL 44), Zacchi P. et al. J Cell Biol. 1998; 140:1039-53 (NPL 45)).

### CITATION LIST

### Non Patent Literature

[NPL 1] Greenlee R T et al. CA Cancer J Clin 2001; 51: 15-36

[NPL 2] Sozzi G. Eur J Cancer 2001; 37 Suppl 7:S63-73

[NPL 3] Schiller J H. et al. N Engl J Med 2002; 346:92-8 (NPL 3)

[NPL 4] Shimada H, et al., Surgery. 2003 May; 133(5):486-94 (NPL 4)

[NPL 5] Mosavi-Jarrahi A & Mohagheghi M A. Asian Pac J Cancer Prey. 2006 July-September; 7(3):375-80

[NPL 6] Thatcher N. et al. Lancet 2005; 366:1527-37

[NPL 7] Shepherd F A. et al. N Engl J Med 2005; 353:123-32

[NPL 8] Daigo Y and Nakamura Y. Gen Thorac Cardiovasc Surg 2008; 56:43-53

[NPL 9] Kikuchi T. et al. Oncogene 2003; 22:2192-205

[NPL 10] Kakiuchi S. et al. Mol Cancer Res 2003; 1:485-99

[NPL 11] Kakiuchi S. et al. Hum Mol Genet 2004; 13:3029-43

[NPL 12] Kikuchi T. et al. Int J Oncol v2006; 28:799-805

[NPL 13] Taniwaki M. et al. Int J Oncol 2006; 29:567-75

[NPL 14] Yamabuki T. et al. Int J Oncol 2006; 28:1375-84

[NPL 15] Suzuki C. et al. Cancer Res 2003; 63:7038-41

[NPL 16] Takahashi K. et al. Cancer Res 2006; 66:9408-19

[NPL 17] Mizukami Y. et al. Cancer Sci 2008; 99:1448-54

[NPL 18] Suzuki C. et al. Cancer Res 2003; 63:7038-41

[NPL 19] Ishikawa N. et al. Clin Cancer Res 2004; 10:8363-70

[NPL 20] Kato T. et al. Cancer Res 2005; 65:5638-46

[NPL 21] Furukawa C. et al. Cancer Res 2005; 65:7102-10

[NPL 22] Ishikawa N. et al. Cancer Res 2005; 65:9176-84

[NPL 23] Suzuki C. et al. Cancer Res 2005; 65:11314-25

[NPL 24] Ishikawa N. et al. Cancer Sci 2006; 97:737-45

[NPL 25] Takahashi K. et al. Cancer Res 2006; 66:9408-19

[NPL 26] Hayama S. et al. Cancer Res 2006; 66:10339-48

[NPL 27] Kato T. et al. Clin Cancer Res 2007; 13:434-42

[NPL 28] Suzuki C. et al. Mol Cancer Ther 2007; 6:542-51

[NPL 29] Yamabuki T. et al. Cancer Res 2007; 67:2517-25

[NPL 30] Hayama S. et al. Cancer Res 2007; 67:4113-22

[NPL 31] Kato T. et al. Cancer Res 2007; 67:8544-53

[NPL 32] Taniwaki M. et al. Clin Cancer Res 2007; 13:6624-31

[NPL 33] Ishikawa N. et al. Cancer Res 2007; 67:11601-11

[NPL 34] Mano Y. et al. Cancer Sci 2007; 98:1902-13

[NPL 35] Suda T. et al. Cancer Sci 2007; 98:1803-8

[NPL 36] Kato T. et al. Clin Cancer Res Res 2008; 14:2363-70

[NPL 37] Mizukami Y. et al. Cancer Sci 2008; 99:1448-54

[NPL 38] Richardson P M and Zon L I. Oncogene 1995; 11: 1139-48

[NPL 39] Bernards A. Biochim Biophys Acta. 2003; 1603: 47-82

[NPL 40] Neuwald A F. Trends Biochem Sci. 1997; 22: 243-4

[NPL 41] Chavrier P and Goud B. Curr Opin Cell Biol. 1999; 11:466-75

[NPL 42] L. Pei. et al. Cancer Res. 2002; 62:5420-24

[NPL 43] Yoshimura S. et al. J Cell Biol. 2007; 178: 363-9

[NPL 44] Lutcke A. et al. J Cell Biol. 1993; 121:553-64

[NPL 45] Zacchi P. et al. J Cell Biol. 1998; 140:1039-53

### SUMMARY OF INVENTION

In the course of screening for novel molecular targets for diagnosis, treatment and prevention of human cancers, genome-wide expression profile analyses of 101 lung cancers was performed on cDNA microarray containing 27,648 genes, coupled with laser microdissection (Kikuchi T, et al. Oncogene. 2003 Apr. 10; 22(14):2192-205; Kikuchi T, et al. Int J Oncol. 2006 April; 28(4):799-805; Kakiuchi S, et al., Mol Cancer Res. 2003 May; 1(7):485-99; Kakiuchi S, et al., Hum Mol Genet. 2004 December 15; 13(24):3029-43. Epub 2004 Oct. 20; Taniwaki M, et al., Int J Oncol. 2006 September; 29(3):567-75). The results demonstrate that the gene encoding the TBC1 domain family, member 7 (TBC1D7) is frequently over-expressed in the great majority of primary lung cancers.

The present invention relates to the cancer-related gene TBC1D7, which is commonly up-regulated in tumors, and strategies for the development of molecular targeted drugs for cancer treatment using TBC1D7.

In one aspect, the present invention provides a method for diagnosing cancer, e.g., a cancer mediated by a TBC1D7, e.g., lung and/or esophageal cancer, using the expression level or biological activity of the TBC1D7 as an index. The present invention also provides a method for predicting the progress of cancer, e.g., lung and/or esophageal cancer therapy in a patient, using the expression level or biological activity of the TBC1D7 as an index. Furthermore, the present invention provides a method for predicting the prognosis of the cancer, e.g., lung and/or esophagus cancer, patient using the expression level or biological activity of the TBC1D7 as an index. In some embodiments, the cancer is mediated or promoted by a TBC1D7. In some embodiments, the cancer is lung and/or esophagus cancer.

In another embodiment, the present invention provides a method for screening an agent for treating or preventing cancers, e.g., a cancer mediated by a TBC1D7, e.g., lung and/or esophageal cancer, using the expression level or biological activity of the TBC1D7 as an index. Particularly, the present invention provides a method for screening an agent for treating or preventing cancers expressing TBC1D7, e.g., lung and/or esophageal cancer, using the interaction between TBC1D7 polypeptide and 14-3-3 zeta polypeptide, between TBC1D7 polypeptide and RAB17 polypeptide or between TBC1D7 polypeptide and TSC1 polypeptide as an index.

In a further embodiment, the present invention provides double-stranded molecules, e.g., siRNA, against the TBC1D7, that were screened by the methods of the present invention. The double-stranded molecules of the present invention are useful for treating or preventing cancers, e.g., a cancer mediated by a TBC1D7 or resulting from overexpression of a TBC1D7, e.g., lung and/or esophageal cancer. The present invention thus further relates to a method for treating cancer including contacting a cancerous cell with an agent screened by the methods of present invention, e.g., siRNA.

### BRIEF DESCRIPTION OF DRAWINGS

Various aspects and applications of the present invention will become apparent to the skilled artisan upon consideration of the brief description of the figures and the detailed description of the present invention and its preferred embodiments which follows:

FIG. 1 depicts the expression of TBC1D7 in lung and esophageal cancer. A, Expression of TBC1D7 in clinical lung and esophageal cancer tissues examined by semiquantitative RT-PCR. B, Expression of TBC1D7 in lung and esophageal cancer cell lines. C, Expression of TBC1D7 protein in lung cancer cell lines examined by western-blotting. D, Expression and subcellular localization of endogenous TBC1D7 protein in lung cancer LC319 cells. E, Northern-blot analysis of the TBC1D7 transcript in 16 normal adult human tissues. A strong signal was observed in testis. F, Immunohistochemical analysis of TBC1D7 protein expressions in 5 normal tissues (heart, lung, liver, kidney, and testis) with those in lung cancers. TBC1D7 expressed abundantly in testis (mainly in nucleus and/or cytoplasm of primary spermatocytes) and lung cancers, but its expression was hardly detectable in the remaining four normal tissues.

FIG. 2 depicts expression of TBC1D7 in normal tissues and association of TBC1D7 overexpression with poor prognosis for NSCLC patients. Association of TBC1D7 expression with poor prognosis. Top panels, Examples for positive and negative staining of TBC1D7 expression in cancer tissues (original magnification X100). Bottom panels, Kaplan-Meier analysis of survival of patients with NSCLC (P=0.0124 by the Log-rank test).

FIG. 3 depicts growth promotive effect of TBC1D7.A, Inhibition of growth of a lung cancer cell line LC319 (left) and A549 (right) by siRNAs against TBC1D7. Top panels, gene knockdown effect on TBC1D7 protein expression in LC319 and A549 cells by two kinds of si-TBC1D7 (si-TBC1D7-#1 and si-TBC1D7-#2) and two control siRNAs (si-EGFP and si-LUC), analyzed by RT-PCR. Middle and bottom panels, colony formation and MTT assays of LC319 and A549 cells transfected with si-TBC1D7s or control siRNAs. Columns, relative absorbance of triplicate assays; bars, SD. B, Flow cytometric analysis of NSCLC cells treated with si-TBC1D7. LC319 cells were transfected with si-TBC1D7-#1 or si-EGFP and collected at 48, 72, and 96 hours after transfection for flow cytometry. C, Enhanced growth of mammalian cells by TBC1D7 overexpression. Assays showing the growth nature of COS-7 cells stably expressing TBC1D7. MTT assays of COS-7 cells stably expressing TBC1D7 comparing their growth with control cells transfected with mock vector. D, Enhanced invasion of mammalian cells by TBC1D7 overexpression. Assays showing the invasive nature of COS-7 cells stably expressing TBC1D7. The number of the invaded COS-7 cells stably expressing TBC1D7 was increased comparing to that of control cells. E, In vivo tumor formation of COS-7 cells caused by TBC1D7 overexpression. All 4 mice that were individually transplanted with COS-7-TBC1D7#A cells had tumors in which overexpression of TBC1D7 protein was confirmed by immunohistochemical analysis. In contrast, no visible tumor was formed in 4 independent mice transplanted with COS-7-Mock-#A cells during 60 days observation. F, Immunohistochemical evaluation of TBC1D7 expression in transplanted tumors at 60 days after cell transplantation (original magnification X 40 and X 200).

FIG. 4 depicts interaction of TBC1D7 with binding proteins. A, Interaction of exogenous TBC1D7 with endogenous 14-3-3 zeta protein in COS-7 cells. Immunoprecipitations were carried out using anti-flag M2 agarose and extracts from COS-7 cells transiently expressing flag-TBC1D7. Immunoprecipitates were subjected to western-blot analysis to detect endogenous 14-3-3. B, Interaction of exogenous TBC1D7 with exogenous RAB 17 protein in COS-7 cells. Immunoprecipitations were carried out using anti-flag M2 agarose and extracts from COS-7 cells transiently expressing flag-TBC1D7 and/or Myc-RAB17. Immunoprecipitates were subjected to western-blot analysis to detect exogenous RAB 17. IB, immunoblotting; IP, immunoprecipitation. C, Expression of TSC1 and TBC1D7 in lung cancer cells. Top panels, Expression of TSC1 and TBC1D7 in lung cancer cell lines examined by semiquantitative RT-PCR. Bottom panels, Expression of TSC1 and TBC1D7 protein in lung cancer cell lines examined by western-blotting. D, E and F, Identification of a TBC1D7-interacting protein TSC1 that stabilizes TBC1D7 protein, and enhanced growth activity in mammalian cells by simultaneous expression of TBC1D7 and TSC1. D, Interaction of endogenous TBC1D7 with endogenous TSC1 protein in lung cancer cells. Immunoprecipitations were carried out using anti-TSC1 antibody and extracts from LC319 cells that express both TBC1D7 and TSC1. Immunoprecipitates were subjected to western-blot analysis to detect endogenous TBC1D7. IB, immunoblotting; IP, immunoprecipitation. E, Effect of TSC1 expression on the levels of TBC1D7 gene and protein. Left panels, The levels of TSC1 and TBC1D7 transcripts and proteins, detected by semiquantitative RT-PCR analysis and western-blot analysis in LC319 cells transfected with si-TSC1. Right panels, The levels of TSC1 and TBC1D7 transcripts and proteins, detected by semiquantitative RT-PCR analysis and western-blot analysis in LC319 cells transfected with TSC1 expression vector. F, The levels of endogenous TSC1 and TBC1D7 proteins, detected by western blot analysis in LC319 cells that were initially transfected with si-TSC1, and were subsequently transfected with TSC1 expression vector 24 hours after the siRNA transfection. Reduced levels of TBC1D7 protein caused by transfection of siRNA against TSC1 were compensated by additional overexpression of exogenous TSC1.

FIG. 5 depicts identification of TSC1-interacting region in TBC1D7 and inhibition of growth of lung cancer cells by dominant-negative peptides of TBC1D7. A, Left panel, Schematic drawing of six N-terminal Flag-tagged TBC1D7 partial protein constructs lacking either or both of the terminal regions. Right panels, Identification of the region in TBC1D7 that binds to TSC1 by immunoprecipitation experiments using LC319 cells. The TBC1D7₁₁₂₋₁₇₁ construct, which corresponds to a center region in TBC domain, was indicated to be TSC1-interacting region. B, Left top panel, Schematic drawing of three cell permeable peptides of TBC1D7 covering TBC1D7₁₁₂₋₁₇₁ that corresponds to the TSC1-interacting region in TBC1D7. B, Right top panels, Reduction of the complex formation between endogenous TSC1 and endogenous TBC1D7 proteins, detected by immunoprecipitation assay in LC319 cells that were treated with the 11R-TBC₁₅₂₋₁₇₁ peptides. C, MTT assay showing growth suppressive effect of 11R-TBC₁₅₂₋₁₇₁ peptides that were introduced into LC319 cells that expressed both TBC1D7 and TSC1 proteins. Bars, SD of triplicate assays. D, Left panels, Expressions of TBC1D7 and TSC1 proteins in lung cancer cell line LC319 and normal human lung fibroblast-derived CCD19Lu cells, examined by western blot analysis. Right panel, MTT assay showing no off-target effect of the 11R-TBC₁₅₂₋₁₇₁ peptides on CCD19Lu cells that scarcely expressed TBC1D7 protein.

FIG. 6 depicts effect of TSC1 expression on mTORC1 pathway in lung cancer LC319 cells. Effects of TSC1 overexpression (Left panels) and TSC1 knockdown (Right panels) on the levels of TBC1D7 protein as well as the levels of phosphorylation of ribosomal protein S6 (rpS6), which is a downstream molecule of mTORC1.

### DESCRIPTION OF EMBODIMENTS

The words “a”, “an”, and “the” as used herein mean “at least one” unless otherwise specifically indicated.

The terms “isolated” and “purified” used in relation with a substance (e.g., polypeptide, antibody, polynucleotide, etc.) indicates that the substance is substantially free from at least one substance that can be included in the natural source. Thus, an isolated or purified antibody refers to antibodies that are substantially free of cellular material for example, carbohydrate, lipid, or other contaminating proteins from the cell or tissue source from which the protein (antibody) is derived, or substantially free of chemical precursors or other chemicals when chemically synthesized. The term “substantially free of cellular material” includes preparations of a polypeptide in which the polypeptide is separated from cellular components of the cells from which it is isolated or recombinantly produced.

Thus, a polypeptide that is substantially free of cellular material includes preparations of polypeptide having less than about 30%, 20%, 10%, or 5% (by dry weight) of heterologous protein (also referred to herein as a “contaminating protein”). When the polypeptide is recombinantly produced, in some embodiments it is also substantially free of culture medium, which includes preparations of polypeptide with culture medium less than about 20%, 10%, or 5% of the volume of the protein preparation. When the polypeptide is produced by chemical synthesis, in some embodiments it is substantially free of chemical precursors or other chemicals, which includes preparations of polypeptide with chemical precursors or other chemicals involved in the synthesis of the protein less than about 30%, 20%, 10%, 5% (by dry weight) of the volume of the protein preparation. That a particular protein preparation contains an isolated or purified polypeptide can be shown, for example, by the appearance of a single band following sodium dodecyl sulfate (SDS)-polyacrylamide gel electrophoresis of the protein preparation and Coomassie Brilliant Blue staining or the like of the gel. In one embodiment, proteins including antibodies of the present invention are isolated or purified.

An “isolated” or “purified” nucleic acid molecule, for example, a cDNA molecule, can be substantially free of other cellular material or culture medium when produced by recombinant techniques, or substantially free of chemical precursors or other chemicals when chemically synthesized. In one embodiment, nucleic acid molecules encoding proteins of the present invention are isolated or purified.

The terms “polypeptide”, “peptide”, and “protein” are used interchangeably herein to refer to a polymer of amino acid residues. The terms apply to amino acid polymers in which one or more amino acid residue is a modified residue, or a non-naturally occurring residue, for example, an artificial chemical mimetic of a corresponding naturally occurring amino acid, as well as to naturally occurring amino acid polymers.

The term “amino acid” refers to naturally occurring and synthetic amino acids, as well as amino acid analogs and amino acid mimetics that similarly functions to the naturally occurring amino acids. Naturally occurring amino acids are those encoded by the genetic code, as well as those modified after translation in cells (e.g., hydroxyproline, gamma-carboxyglutamate, and O-phosphoserine). The phrase “amino acid analog” refers to compounds that have the same basic chemical structure (an alpha carbon bound to a hydrogen, a carboxy group, an amino group, and an R group) as a naturally occurring amino acid but have a modified R group or modified backbones (e.g., homoserine, norleucine, methionine, sulfoxide, methionine methyl sulfonium). The phrase “amino acid mimetic” refers to chemical compounds that have different structures but similar functions to general amino acids.

Amino acids can be referred to herein by their commonly known three letter symbols or the one-letter symbols recommended by the IUPAC-IUB Biochemical Nomenclature Commission. The terms “polynucleotides”, “oligonucleotide”, “nucleotides”, “nucleic acids”, and “nucleic acid molecules” are used interchangeably unless otherwise specifically indicated and are similarly to the amino acids referred to by their commonly accepted single-letter codes. Similar to the amino acids, they encompass both naturally-occurring and non-naturally occurring nucleic acid polymers. The polynucleotide, oligonucleotide, nucleotides, nucleic acids, or nucleic acid molecules can be composed of DNA, RNA or a combination thereof.

As used herein, the term “biological sample” refers to a whole organism or a subset of its tissues, cells or component parts (e.g., body fluids, including but not limited to blood, mucus, lymphatic fluid, synovial fluid, cerebrospinal fluid, saliva, amniotic fluid, amniotic cord blood, urine, vaginal fluid and semen). “Biological sample” further refers to a homogenate, lysate, extract, cell culture or tissue culture prepared from a whole organism or a subset of its cells, tissues or component parts, or a fraction or portion thereof. Lastly, “biological sample” refers to a medium, for example, a nutrient broth or gel in which an organism has been propagated, which contains cellular components, for example, proteins or polynucleotides.

The nucleotide sequence of human TBC1D7 gene is shown in SEQ ID NO: 1 and is also available as GenBank Accession No. NM_{—}016495. Herein, the phrase “TBC1D7 gene” encompasses the human TBC1D7 gene as well as those of other animals including non-human primate, mouse, rat, dog, cat, horse, and cow but is not limited thereto, and includes allelic mutants and genes found in other animals as corresponding to the TBC1D7 gene. The amino acid sequence encoded by the human TBC1D7 gene is shown as SEQ ID NO: 2 and is also available as GenBank Accession No. NP_{—}057579.1. In the present invention, the polypeptide encoded by the TBC1D7 gene is referred to as “TBC1D7”, and sometimes as “TBC1D7 polypeptide” or “TBC1D7 protein”.

According to an aspect of the present invention, functional equivalents are also included in the TBC1D7. Herein, a “functional equivalent” of a protein is a polypeptide that has a biological activity equivalent to the protein. Namely, any polypeptide that retains at least one biological activity of TBC1D7 can be used as such a functional equivalent in the present invention. For example, the functional equivalent of TBC1D7 retains promoting activity of cell proliferation and/or invasion activity. In addition, the biological activity of TBC1D7 contains binding activity to RAB17 (GenBank Accession No. NM_{—}022449.2: SEQ ID NO: 12), 14-3-3 zeta (GenBank Accession No. NM_{—}003406, SEQ ID NO: 14) or TSC1 (GenBank Accession No. NM_{—}001143964.1: SEQ ID NO: 45). The functional equivalent of TBC1D7 can contain a RAB17 binding region, 14-3-3 zeta binding region and/or TSC1 binding region (e. g., TBC152-171: SEQ ID NO: 28).

Functional equivalents of TBC1D7 include those wherein one or more amino acids, e.g., 1-5 amino acids, e.g., up to 5% of amino acids, are substituted, deleted, added, or inserted to the natural occurring amino acid sequence of the TBC1D7 protein. Alternatively, a functional equivalent may be a polypeptide composed an amino acid sequence having at least about 80% homology (also referred to as sequence identity) to the sequence of the respective protein, more preferably at least about 90% to 95% homology, often about 96%, 97%, 98% or 99% homology to SEQ ID NO: 2.

Generally, it is known that modifications of one or more amino acid in a protein do not influence the function of the protein (Mark D F, et al., Proc Natl Acad Sci USA. 1984 September; 81(18):5662-6; Zoller M J & Smith M. Nucleic Acids Res. 1982 Oct. 25; 10(20):6487-500; Wang A, et al., Science. 1984 Jun. 29; 224(4656):1431-3; Dalbadie-McFarland G, et al., Proc Natl Acad Sci USA. 1982 November; 79(21):6409-13). One of skill in the art will recognize that individual additions, deletions, insertions, or substitutions to an amino acid sequence which alters a single amino acid or a small percentage of amino acids is a “conservative modification” wherein the alteration of a protein results in a protein with similar functions.

Examples of properties of amino acid side chains are hydrophobic amino acids (alanine, isoleucine, leucine, methionine, phenylalanine, proline, tryptophan, tyrosine, valine), hydrophilic amino acids (arginine, aspartic acid, aspargin, cystein, glutamic acid, glutamine, glycine, histitidine, lysine, serine, threonine), and side chains having the following functional groups or characteristics in common: an aliphatic side-chain (glycine, alanine, valine, leucine, isoleucine, praline); a hydroxyl group containing side-chain (serine, threonine, tyrosine); a sulfur atom containing side-chain (C, M); a carboxylic acid and amide containing side-chain (aspartic acid, aspargine, glutamic acid, glutamine); a base containing side-chain (arginine, lysine, histidine); and an aromatic containing side-chain (histidine, phenylalanine, tyrosine, tryptophan). Furthermore, conservative substitution tables providing functionally similar amino acids are well known in the art. For example, the following eight groups each contain amino acids that are conservative substitutions for one another:
(1) Alanine (A), Glycine (G);
(2) Aspartic acid (D), Glutamic acid (E);
(3) Aspargine (N), Glutamine (Q);
(4) Arginine (R), Lysine (K);
(5) Isoleucine (I), Leucine (L), Methionine (M), Valine (V);
(6) Phenylalanine (F), Tyrosine (Y), Tryptophan (W);
(7) Serine (S), Threonine (T); and
(8) Cystein (C), Methionine (M)
   (see, e.g., Thomas E. Creighton, Proteins Publisher: New York: W.H. Freeman, c1984).

Such conservatively modified polypeptides are included in the TBC1D7 protein. However, the present invention is not restricted thereto and the TBC1D7 protein includes non-conservative modifications so long as they retain any one of the biological activity of the TBC1D7 protein. The number of amino acids to be mutated in such a modified protein is generally 10 amino acids or fewer, for example, 6 amino acids or fewer, for example, 3 amino acids or fewer.

An example of a protein modified by addition of one or more amino acids residues is a fusion protein of the TBC1D7 protein. Fusion proteins include fusions of the TBC1D7 protein and other peptides or proteins, which also can be used in the present invention. Fusion proteins can be made by techniques well known to a person skilled in the art, for example, by linking the DNA encoding the TBC1D7 gene with a DNA encoding other peptides or proteins, so that the frames match, inserting the fusion DNA into an expression vector and expressing it in a host. There is no restriction as to the peptides or proteins fused to the TBC1D7 protein so long as the resulting fusion protein retains any one of the objective biological activity of the TBC1D7 proteins.

Known peptides that can be used as peptides to be fused to the TBC1D7 protein include, for example, FLAG (Hopp T P, et al., Biotechnology 6: 1204-10 (1988)), 6× His containing six His (histidine) residues, 10× His, Influenza agglutinin (HA), human c-myc fragment, VSP-GP fragment, p18HIV fragment, T7-tag, HSV-tag, E-tag, SV40T antigen fragment, lck tag, alpha-tubulin fragment, B-tag, Protein C fragment, and the like. Examples of proteins that can be fused to a protein of the invention include GST (glutathione-S-transferase), Influenza agglutinin (HA), immunoglobulin constant region, beta-galactosidase, MBP (maltose-binding protein), and such.

Furthermore, the modified proteins do not exclude polymorphic variants, interspecies homologues, and those encoded by alleles of these proteins.

Methods known in the art to isolate functional equivalent proteins include, for example, hybridization techniques (Sambrook and Russell, Molecular Cloning: A Laboratory Manual, 3rd ed., Cold Spring Harbor Lab. Press, 2001). One skilled in the art can readily isolate a DNA having high homology (i.e., sequence identity) with a whole or part of the human TBC1D7 DNA sequences (e.g., SEQ ID NO: 1) encoding the human TBC1D7 protein, and isolate functional equivalent proteins to the human TBC1D7 protein from the isolated DNA. Thus, the proteins used for the present invention include those that are encoded by DNA that hybridize under stringent conditions with a whole or part of the DNA sequence encoding the human TBC1D7 protein and are functional equivalent to the human TBC1D7 protein. These proteins include mammal homologues corresponding to the protein derived from human or mouse (for example, a protein encoded by a monkey, rat, rabbit or bovine gene). In isolating a cDNA highly homologous to the DNA encoding the human TBC1D7 gene from lung or esophagus cancer tissue or cell line, or tissues from testis can be used.

The conditions of hybridization for isolating a DNA encoding a protein functional equivalent to the human TBC1D7 gene can be routinely selected by a person skilled in the art. The phrase “stringent (hybridization) conditions” refers to conditions under which a nucleic acid molecule will hybridize to its target sequence, typically in a complex mixture of nucleic acids, but not detectably to other sequences. Stringent conditions are sequence-dependent and will differ under different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10 degrees Centigrade lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions can also be achieved with the addition of destabilizing agents for example, formamide. For selective or specific hybridization, a positive signal is at least two times of background, for example, 10 times of background hybridization.

For example, hybridization can be performed by conducting prehybridization at 68 degrees C. for 30 min or longer using “Rapid-hyb buffer” (Amersham LIFE SCIENCE), adding a labeled probe, and warming at 68 degrees C. for 1 h or longer. The following washing step can be conducted, for example, in a low stringent condition. A low stringent condition is, for example, 42 degrees C., 2×SSC, 0.1% SDS, for example, 50 degrees C., 2×SSC, 0.1% SDS. In some embodiments, high stringent condition is used. A high stringent condition is, for example, washing 3 times in 2×SSC, 0.01% SDS at room temperature for 20 min, then washing 3 times in 1×SSC, 0.1% SDS at 37 degrees C. for 20 min, and washing twice in 1×SSC, 0.1% SDS at 50 degrees C. for 20 min. However, several factors for example, temperature and salt concentration can influence the stringency of hybridization and one skilled in the art can suitably select the factors to achieve the requisite stringency.

In place of hybridization, a gene amplification method, for example, the polymerase chain reaction (PCR) method, can be utilized to isolate a DNA encoding a protein functional equivalent to the human TBC1D7 gene, using a primer synthesized based on the sequence information of the DNA (SEQ ID NO: 1) encoding the human TBC1D7 protein (SEQ ID NO: 2), examples of primer sequences are pointed out in (b) Semiquantitative RT-PCR in [EXAMPLE 1].

Proteins that are functional equivalent to the human TBC1D7 protein encoded by the DNA isolated through the above hybridization techniques or gene amplification techniques, normally have a high homology (also referred to as sequence identity) to the amino acid sequence of the human TBC1D7 protein. “High homology” (also referred to as “high sequence identity”) typically refers to the degree of identity between two optimally aligned sequences (either polypeptide or polynucleotide sequences). Typically, high homology or sequence identity refers to homology of 40% or higher, for example, 60% or higher, for example, 80% or higher, for example, 85%, 90%, 95%, 98%, 99%, or higher. The degree of homology or identity between two polypeptide or polynucleotide sequences can be determined by following the algorithm (Wilbur W J & Lipman D J. Proc Natl Acad Sci USA. 1983 February; 80 (3):726-30).

Additional examples of algorithms that are suitable for determining percent sequence identity and sequence similarity are the BLAST and BLAST 2.0 algorithms, which are described (Altschul S F, et al., J Mol Biol. 1990 Oct. 5; 215 (3):403-10; Nucleic Acids Res. 1997 Sep. 1; 25(17):3389-402). Software for performing BLAST analyses is publicly available through the National Center for Biotechnology Information (on the worldwide web at ncbi.nlm.nih.gov/). The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them.

The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached.

The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (Henikoff S & Henikoff J G. Proc Natl Acad Sci USA. 1992 Nov. 15; 89(22):10915-9).

A protein useful in the context of the present invention can have variations in amino acid sequence, molecular weight, isoelectric point, the presence or absence of sugar chains, or form, depending on the cell or host used to produce it or the purification method utilized. Nevertheless, so long as it has any one of the biological activity of the TBC1D7 protein (SEQ ID NO: 2), it is useful in the present invention.

The present invention also encompasses the use of partial peptides of the TBC1D7 protein. A partial peptide has an amino acid sequence specific to the protein of the TBC1D7 protein and consists of less than about 400 amino acids, usually less than about 200 and often less than about 100 amino acids, and at least about 7 amino acids, for example, about 8 amino acids or more, for example, about 9 amino acids or more.

A partial peptide used for the screening methods of the present invention suitably contains at least a binding domain of TBC1D7. Furthermore, a partial TBC1D7 peptide used for the screenings of the present invention suitably contains 14-3-3 zeta binding region, RAB17 binding region. Such partial peptides are also encompassed by the phrase “functional equivalent” of the TBC1D7 protein.

The polypeptide or fragments used for the present method can be obtained from nature as naturally occurring proteins via conventional purification methods or through chemical synthesis based on the selected amino acid sequence. For example, conventional peptide synthesis methods that can be adopted for the synthesis include:

(1) Peptide Synthesis, Interscience, New York, 1966;

(2) The Proteins, Vol. 2, Academic Press, New York, 1976;

(3) Peptide Synthesis (in Japanese), Maruzen Co., 1975;

(4) Basics and Experiment of Peptide Synthesis (in Japanese), Maruzen Co., 1985;

(5) Development of Pharmaceuticals (second volume) (in Japanese), Vol. 14 (peptide synthesis), Hirokawa, 1991;

(6) WO99/67288; and

(7) Barany G. & Merrifield R. B., Peptides Vol. 2, “Solid Phase Peptide Synthesis”, Academic Press, New York, 1980, 100-118.

Alternatively, the protein can be obtained adopting any known genetic engineering methods for producing polypeptides (e.g., Morrison D A., et al., J Bacteriol. 1977 October; 132(1):349-51; Clark-Curtiss J E & Curtiss R 3rd. Methods Enzymol. 1983; 101:347-62). For example, a suitable vector including a polynucleotide encoding the objective protein in an expressible form (e.g., downstream of a regulatory sequence including a promoter) is prepared, transformed into a suitable host cell, and then the host cell is cultured to produce the protein. More specifically, a gene encoding the TBC1D7 polypeptide is expressed in host (e.g., animal) cells and such by inserting the gene into a vector for expressing foreign genes, for example, pSV2neo, pcDNA I, pcDNA3.1, pCAGGS, or pCD8.

A promoter can be used for the expression. Any commonly used promoters can be employed including, for example, the SV40 early promoter (Rigby in Williamson (ed.), Genetic engineering, vol. 3. Academic Press, London, 1982, 83-141), the EF-alpha promoter (Kim D W, et al. Gene. 1990 Jul. 16; 91(2):217-23), the CAG promoter (Niwa H, et al., Gene. 1991 Dec. 15; 108(2):193-9), the RSV LTR promoter (Cullen B R. Methods Enzymol. 1987; 152:684-704), the SR alpha promoter (Takebe Y, et al., Mol Cell Biol. 1988 January; 8(1):466-72), the CMV immediate early promoter (Seed B & Aruffo A. Proc Natl Acad Sci USA. 1987 May; 84(10):3365-9), the SV40 late promoter (Gheysen D & Fiers W. J Mol Appl Genet. 1982; 1(5):385-94), the Adenovirus late promoter (Kaufman R J, et al., Mol Cell Biol. 1989 March; 9(3):946-58), the HSV TK promoter, and the like.

The introduction of the vector into host cells to express the TBC1D7 gene can be performed according to any methods, for example, the electroporation method (Chu G, et al., Nucleic Acids Res. 1987 Feb. 11; 15(3):1311-26), the calcium phosphate method (Chen C & Okayama H. Mol Cell Biol. 1987 August; 7(8):2745-52), the DEAE dextran method (Lopata M A, et al., Nucleic Acids Res. 1984 Jul. 25; 12(14):5707-17; Sussman D J & Milman G. Mol Cell Biol. 1984 August; 4(8):1641-3), the Lipofectin method (Derijard B, et al., Cell. 1994 Mar. 25; 76(6):1025-37; Lamb B T, et al., Nat Genet. 1993 September; 5(1):22-30; Rabindran S K, et al., Science. 1993 Jan. 8; 259(5092):230-4), and such.

The TBC1D7 proteins can also be produced in vitro adopting an in vitro translation system. In the context of the present invention, the phrase “TBC1D7 gene” encompasses polynucleotides that encode the human TBC1D7 protein or any of the functional equivalents of the human TBC1D7 protein.

The TBC1D7 gene can be obtained from nature as naturally occurring proteins via conventional cloning methods or through chemical synthesis based on the selected nucleotide sequence. Methods for cloning genes using cDNA libraries and such are well known in the art.

(2) Antibody

The terms “antibody” as used herein is intended to include immunoglobulins and fragments thereof which are specifically reactive to the designated protein or peptide thereof. An antibody can include human antibodies, primatized antibodies, chimeric antibodies, bispecific antibodies, humanized antibodies, antibodies fused to other proteins or radiolabels, and antibody fragments. Furthermore, an antibody herein is used in the broadest sense and specifically covers intact monoclonal antibodies, polyclonal antibodies, multispecific antibodies (e.g. bispecific antibodies) formed from at least two intact antibodies, and antibody fragments so long as they exhibit the desired biological activity. An “antibody” indicates all classes (e.g. IgA, IgD, IgE, IgG and IgM).

The subject invention uses antibodies against TBC1D7 protein. These antibodies can be useful for diagnosing lung cancer or eshopageal cancer. Furthermore, the subject invention uses antibodies against TBC1D7 polypeptide or partial peptide of them, especially antibodies against RAB17 binding region of TBC1D7 polypeptide, 14-3-3 zeta binding region of TBC1D7 polypeptide, or TSC1 binding region of TBC1D7 polypeptide (e. g. SEQ ID NO:28).

These antibodies can be useful for inhibiting and/or blocking an interaction, e.g.

binding, between TBC1D7 polypeptide and RAB17 polypeptide or an interaction, e.g. binding, between TBC1D7 polypeptide, 14-3-3 zeta, e.g. binding, between TBC1D7 polypeptide, TSC1. polypeptide and can be useful for treating and/or preventing cancer (over)expressing TBC1D7, e.g. lung cancer or eshopageal cancer. Alternatively, the subject invention also uses antibodies against RAB 17 polypeptide, 14-3-3 zeta polypeptide, TSC1 or partial peptide of them, e.g. TBC1D7 binding region of them such as SEQ ID NO: 28. These antibodies will be provided by known methods. Exemplary techniques for the production of the antibodies used in accordance with the present invention are described.

(i) Polyclonal Antibodies

Polyclonal antibodies can be raised in animals by multiple subcutaneous (sc) or intraperitoneal (ip) injections of the relevant antigen and an adjuvant. Conjugating the relevant antigen to a protein that is immunogenic in the species to be immunized finds use, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOC12, or R′N═C═NR, where R and R are different alkyl groups.

Animals are immunized against the antigen, immunogenic conjugates, or derivatives by combining, e.g. 100 micro g or 5 micro g of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. In some embodiments, the animal is boosted with the conjugate of the same antigen, but conjugated to a different protein and/or through a different cross-linking reagent.

Conjugates also can be made in recombinant cell culture as protein fusions. Also, aggregating agents for example, alum are suitably used to enhance the immune response.

(ii) Monoclonal Antibodies

Monoclonal antibodies are obtained from a population of substantially homogeneous antibodies, i.e., the individual antibodies including the population are identical except for possible naturally occurring mutations that may be present in minor amounts. Thus, the modifier “monoclonal” indicates the character of the antibody as not being a mixture of discrete antibodies.

For example, the monoclonal antibodies can be made using the hybridoma method first described by Kohler G & Milstein C. Nature. 1975 Aug. 7; 256 (5517):495-7, or can be made by recombinant DNA methods (U.S. Pat. No. 4,816,567).

In the hybridoma method, a mouse or other appropriate host animal, for example, a hamster, is immunized as hereinabove described to elicit lymphocytes that produce or are capable of producing antibodies that will specifically bind to the protein used for immunization. Alternatively, lymphocytes can be immunized in vitro. Lymphocytes then are fused with myeloma cells using a suitable fusing agent, for example, polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that can contain one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

In some embodiments, myeloma cells are those that fuse efficiently, support stable high-level production of antibody by the selected antibody-producing cells, and are sensitive to a medium for example, HAT medium. Exemplary myeloma cell lines include murine myeloma lines, for example, those derived from MOPC-21 and MPC-11 mouse tumors available from the Salk Institute Cell Distribution Center, San Diego, Calif. USA, and SP-2 or X63-Ag8-653 cells available from the American Type Culture Collection, Manassas, Va., USA. Human myeloma and mouse-human heteromyeloma cell lines also have been described for the production of human monoclonal antibodies (Kozbor D, et al., J Immunol. 1984 December; 133(6):3001-5; Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. In some embodiments, the binding specificity of monoclonal antibodies produced by hybridoma cells is determined by immunoprecipitation or by an in vitro binding assay, for example, radioimmunoassay (RIA) or enzyme-linked immunoabsorbent assay (ELISA).

The binding affinity of the monoclonal antibody can, for example, be determined by the 30 Scatchard analysis of Munson P J & Rodbard D. Anal Biochem. 1980 Sep. 1; 107(1):220-39. After hybridoma cells are identified that produce antibodies of the desired specificity, affinity, and/or activity, the clones can be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPML-1640 medium. In addition, the hybridoma cells can be grown in vivo as ascites tumors in an animal.

The monoclonal antibodies secreted by the subclones are suitably separated from the culture medium, ascites fluid, or serum by conventional immunoglobulin purification procedures for example, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a source of such DNA. Once isolated, the DNA can be placed into expression vectors, which are then transfected into host cells for example, E. coli cells, simian COS cells, Chinese Hamster Ovary (CHO) cells, or myeloma cells that do not otherwise produce immunoglobulin protein, to obtain the synthesis of monoclonal antibodies in the recombinant host cells. Review articles on recombinant expression in bacteria of DNA encoding the antibody include Skerra A. Curr Opin Immunol. 1993 April; 5 (2):256-62 and Pluckthun A. Immunol Rev. 1992 December; 130:151-88.

Another method of generating specific antibodies, or antibody fragments, reactive against TBC1D7 protein is to screen expression libraries encoding immunoglobulin genes, or portions thereof, expressed in bacteria with TBC1D7 protein or peptide. For example, complete Fab fragments, VH regions and Fv regions can be expressed in bacteria using phage expression libraries. See for example, Ward E S, et al., Nature. 1989 Oct. 12; 341(6242):544-6; Huse W D, et al., Science. 1989 Dec. 8; 246(4935):1275-81; and McCafferty J, et al., Nature. 1990 Dec. 6; 348(6301):552-4. Screening such libraries with, TBC1D7 protein, e.g. TBC1D7 peptides, can identify immunoglobulin fragments reactive with the TBC1D7 protein. Alternatively, the SCID-humouse (available from Genpharm) can be used to produce antibodies or fragments thereof.

In a further embodiment, antibodies or antibody fragments can be isolated from antibody phage libraries generated using the techniques described in McCafferty J, et al., Nature. 1990 Dec. 6; 348(6301):552-4; Clackson T, et al., Nature. 1991 Aug. 15; 352(6336):624-8; and Marks J D, et al., J MoL BioL, 222: 581-597 (1991) J Mol Biol. 1991 Dec. 5; 222(3):581-97 describe the isolation of murine and human antibodies, respectively, using phage libraries. Subsequent publications describe the production of high affinity (nM range) human antibodies by chain shuffling (Marks J D, et al., Biotechnology (NY). 1992 July; 10(7):779-83), as well as combinatorial infection and in vivo recombination as a strategy for constructing very large phage libraries (Waterhouse P, et al., Nucleic Acids Res. 1993 May 11; 21(9):2265-6). Thus, these techniques are viable alternatives to traditional monoclonal antibody hybridoma techniques for isolation of monoclonal antibodies.

The DNA also can be modified, for example, by substituting the coding sequence for human heavy-and light-chain constant domains in place of the homologous murine sequences (U.S. Pat. No. 4,816,567; Morrison S L, et al., Proc Natl Acad Sci USA. 1984 November; 81(21): 6851-5), or by covalently joining to the immunoglobulin coding sequence all or part of the coding sequence for a non-immunoglobulin polypeptide.

Typically, such non-immunoglobulin polypeptides are substituted for the constant domains of an antibody, or they are substituted for the variable domains of one antigencombining site of an antibody to create a chimeric bivalent antibody including one antigen-combining site having specificity for an antigen and another antigen-combining site having specificity for a different antigen.

(iii) Humanized Antibodies

Methods for humanizing non-human antibodies have been described in the art. In some embodiments, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones P T, et al., Nature. 1986 May 29-Jun. 4; 321(6069):522-5; Riechmann L, et al., Nature. 1988 Mar. 24; 332(6162):323-7; Verhoeyen M, et al., Science. 1988 Mar. 25; 239(4847):1534-6), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so called “best-fit” method, the sequence of the variable domain of a rodent antibody is screened against the entire library of known human variable-domain sequences. The human sequence which is closest to that of the rodent is then accepted as the human framework region (FR) for the humanized antibody (Sims M J, et al., J Immunol. 1993 Aug. 15; 151(4):2296-308; Chothia C & Lesk A M. J Mol Biol. 1987 Aug. 20; 196(4):901-17). Another method uses a particular framework region derived from the consensus sequence of all human antibodies of a particular subgroup of light or heavy chains. The same framework can be used for several different humanized antibodies (Carter P, et al., Proc Natl Acad Sci USA. 1992 May 15; 89(10):4285-9; Presta L G, et al., J Immunol. 1993 Sep. 1; 151(5):2623-32).

It is further important that antibodies be humanized with retention of high affinity for the antigen and other favorable biological properties. To achieve this goal, in some embodiments, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, for example, increased affinity for the target antigen, is achieved. In general, the hypervariable region residues are directly and most substantially involved in influencing antigen binding.

(iv) Human Antibodies

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ line mutant mice will result in the production of human antibodies upon antigen challenge. See, e.g., Jakobovits A, et al., Proc Natl Acad Sci USA. 1993 Mar. 15; 90(6):2551-5; Nature. 1993 Mar. 18; 362(6417):255-8; Bruggemann M, et al., Year Immunol. 1993; 7:33-40; and U.S. Pat. Nos. 5,591,669; 5,589,369 and 5,545,807.

Alternatively, phage display technology (McCafferty J, et al., Nature. 1990 Dec. 6; 348(6301):552-4) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, for example, M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson K S & Chiswell D J. Curr Opin Struct Biol. 1993; 3:564-71. Several sources of V-gene segments can be used for phage display.

Clackson T, et al., Nature. 1991 Aug. 15; 352(6336):624-8 isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self antigens) can be isolated essentially following the techniques described by Marks J D, et al., J Mol Biol. 1991 Dec. 5; 222(3):581-97, or Griffiths A D, et al., EMBO J. 1993 February; 12(2):725-34. See, also, U.S. Pat. Nos. 5,565,332 and 5,573,905.

Human antibodies can also be generated by in vitro activated B cells (see U.S. Pat. Nos. 20 5,567,610 and 5,229,275).

(v) Non-Antibody Binding Proteins

The present invention also contemplates non-antibody binding proteins against CX proteins, including against the N-terminal portion of EPHA7. The terms “non-antibody binding protein” or “non-antibody ligand” or “antigen binding protein” interchangeably refer to antibody mimics that use non-immunoglobulin protein scaffolds, including adnectins, avimers, single chain polypeptide binding molecules, and antibody-like binding peptidomimetics, as discussed in more detail below.

Other compounds have been developed that target and bind to targets in a manner similar to antibodies. Certain of these “antibody mimics” use non-immunoglobulin protein scaffolds as alternative protein frameworks for the variable regions of antibodies.

For example, Ladner et al. (U.S. Pat. No. 5,260,203) describe single polypeptide chain binding molecules with binding specificity similar to that of the aggregated, but molecularly separate, light and heavy chain variable region of antibodies. The single-chain binding molecule contains the antigen binding sites of both the heavy and light variable regions of an antibody connected by a peptide linker and will fold into a structure similar to that of the two peptide antibody. The single-chain binding molecule displays several advantages over conventional antibodies, including, smaller size, greater stability and are more easily modified.

Ku et al. (Proc Natl Acad Sci USA 92(14):6552-6556 (1995)) discloses an alternative to antibodies based on cytochrome b562. Ku et al. (1995) generated a library in which two of the loops of cytochrome b562 were randomized and selected for binding against bovine serum albumin. The individual mutants were found to bind selectively with BSA similarly with anti-BSA antibodies.

Lipovsek et al. (U.S. Pat. Nos. 6,818,418 and 7,115,396) discloses an antibody mimic featuring a fibronectin or fibronectin-like protein scaffold and at least one variable loop. Known as Adnectins, these fibronectin-based antibody mimics exhibit many of the same characteristics of natural or engineered antibodies, including high affinity and specificity for any targeted ligand. Any technique for evolving new or improved binding proteins can be used with these antibody mimics.

The structure of these fibronectin-based antibody mimics is similar to the structure of the variable region of the IgG heavy chain. Therefore, these mimics display antigen binding properties similar in nature and affinity to those of native antibodies. Further, these fibronectin-based antibody mimics exhibit certain benefits over antibodies and antibody fragments. For example, these antibody mimics do not rely on disulfide bonds for native fold stability, and are, therefore, stable under conditions which would normally break down antibodies. In addition, since the structure of these fibronectin-based antibody mimics is similar to that of the IgG heavy chain, the process for loop randomization and shuffling can be employed in vitro that is similar to the process of affinity maturation of antibodies in vivo. Beste et al. (Proc Natl Acad Sci USA 96(5):1898-1903 (1999)) discloses an antibody mimic based on a lipocalin scaffold (Anticalin(registered trademark)). Lipocalins are composed of a beta-barrel with four hypervariable loops at the terminus of the protein. Beste (1999), subjected the loops to random mutagenesis and selected for binding with, for example, fluorescein. Three variants exhibited specific binding with fluorescein, with one variant showing binding similar to that of an anti-fluorescein antibody. Further analysis revealed that all of the randomized positions are variable, indicating that Anticalin(registered trademark) would be suitable to be used as an alternative to antibodies.

Anticalins(registered trademark) are small, single chain peptides, typically between 160 and 180 residues, which provide several advantages over antibodies, including decreased cost of production, increased stability in storage and decreased immunological reaction.

Hamilton et al. (U.S. Pat. No. 5,770,380) discloses a synthetic antibody mimic using the rigid, non-peptide organic scaffold of calixarene, attached with multiple variable peptide loops used as binding sites. The peptide loops all project from the same side geometrically from the calixarene, with respect to each other. Because of this geometric confirmation, all of the loops are available for binding, increasing the binding affinity to a ligand. However, in comparison to other antibody mimics, the calixarene-based antibody mimic does not consist exclusively of a peptide, and therefore it is less vulnerable to attack by protease enzymes. Neither does the scaffold consist purely of a peptide, DNA or RNA, meaning this antibody mimic is relatively stable in extreme environmental conditions and has a long life span. Further, since the calixarene-based antibody mimic is relatively small, it is less likely to produce an immunogenic response.

Murali et al. (Cell Mol Biol. 49(2):209-216 (2003)) discusses a methodology for reducing antibodies into smaller peptidomimetics, they term “antibody like binding peptidomimetics” (ABiP) which can also be useful as an alternative to antibodies.

Silverman et al. (Nat Biotechnol. (2005), 23: 1556-1561) discloses fusion proteins that are single-chain polypeptides including multiple domains termed “avimers.” Developed from human extracellular receptor domains by in vitro exon shuffling and phage display the avimers are a class of binding proteins somewhat similar to antibodies in their affinities and specificities for various target molecules. The resulting multidomain proteins can include multiple independent binding domains that can exhibit improved affinity (in some cases sub-nanomolar) and specificity compared with single-epitope binding proteins. Additional details concerning methods of construction and use of avimers are disclosed, for example, in US Pat. App. Pub. Nos. 20040175756, 20050048512, 20050053973, 20050089932 and 20050221384.

In addition to non-immunoglobulin protein frameworks, antibody properties have also been mimicked in compounds including RNA molecules and unnatural oligomers (e.g., protease inhibitors, benzodiazepines, purine derivatives and beta-turn mimics) all of which are suitable for use with the present invention.

As known in the art, aptamers are macromolecules composed of nucleic acid that bind tightly to a specific molecular target. Tuerk and Gold (Science. 249:505-510 (1990)) discloses SELEX (Systematic Evolution of Ligands by Exponential Enrichment) method for selection of aptamers. In the SELEX method, a large library of nucleic acid molecules {e.g., 10¹⁵ different molecules) is produced and/or screened with the target molecule. Isolated aptamers can then be further refined to eliminate any nucleotides that do not contribute to target binding and/or aptamer structure (i.e., aptamers truncated to their core binding domain). See, e.g., Jayasena, 1999, Clin. Chem. 45:1628-1650 for review of aptamer technology.

Although the construction of test agent/compound libraries is well known in the art, herein below, additional guidance in identifying test agents or compounds and construction libraries of such agents or compounds for the present screening methods are provided.

(vi) Antibody Fragments

Various techniques have been developed for the production of antibody fragments. Traditionally, these fragments were derived via proteolytic digestion of intact antibodies (see, e.g., Morimoto K & Inouye K. J Biochem Biophys Methods. 1992 March; 24(1-2):107-17; Brennan M, et al., Science. 1985 Jul. 5; 229(4708):81-3). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′) 2 fragments (Carter P, et al., Biotechnology (NY). 1992 February; 10(2):163-7). According to another approach, F(ab′) 2 fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. Nos. 5,571,894 and 5,587,458. The antibody fragment can also be a “linear antibody”, e.g., as described in U.S. Pat. No. 5,641,870 for example. Such linear antibody fragments can be monospecific or bispecific.

(vii) Selecting the Antibody or Antibody Fragment

The antibody or antibody fragment which prepared by aforementioned method is selected by detecting affinity of CX genes expressing cells like cancers cell. Unspecific binding to these cells is blocked by treatment with PBS containing 3% BSA for 30 min at room temperature. Cells are incubated for 60 min at room temperature with candidate antibody or antibody fragment. After washing with PBS, the cells are stained by FITC-conjugated secondary antibody for 60 min at room temperature and detected by using fluorometer. Alternatively, a biosensor using the surface plasmon resonance phenomenon can be used as a mean for detecting or quantifying the antibody or antibody fragment in the present invention. The antibody or antibody fragment which can detect the CX peptide on the cell surface is selected in the presence invention.

(3) Double-Stranded Molecule

The term “polynucleotide” and “oligonucleotide” are used interchangeably herein unless otherwise specifically indicated and are referred to by their commonly accepted single-letter codes. The terms apply to nucleic acid (nucleotide) polymers in which one or more nucleic acids are linked by ester bonding. The polynucleotide or oligonucleotide can be composed of DNA, RNA or a combination thereof.

As use herein, the term “isolated double-stranded molecule” refers to a nucleic acid molecule that inhibits expression of a target gene including, for example, short interfering RNA (siRNA; e.g., double-stranded ribonucleic acid (dsRNA) or small hairpin RNA (shRNA)) and short interfering DNA/RNA (siD/R-NA; e.g. double-stranded chimera of DNA and RNA (dsD/R-NA) or small hairpin chimera of DNA and RNA (shD/R-NA)).

As use herein, the term “siRNA” refers to a double-stranded RNA molecule which prevents translation of a target mRNA. Standard techniques of introducing siRNA into the cell are used, including those in which DNA is a template from which RNA is transcribed. The siRNA includes a ribonucleotide corresponding to a sense nucleic acid sequence of TBC1D7 gene (also referred to as “sense strand”), a ribonucleotide corresponding to an antisense nucleic acid sequence of TBC1D7 gene (also referred to as “antisense strand”) or both. The siRNA can be constructed such that a single transcript has both the sense and complementary antisense nucleic acid sequences of the target gene, e.g., a hairpin. The siRNA can either be a dsRNA or shRNA. As used herein, the term “dsRNA” refers to a construct of two RNA molecules including complementary sequences to one another and that have annealed together via the complementary sequences to form a double-stranded RNA molecule. The sequence of two strands can include not only the “sense” or “antisense” RNAs selected from a protein coding sequence of target gene sequence, but also RNA molecule having a nucleotide sequence selected from non-coding region of the target gene.

The term “shRNA”, as used herein, refers to an siRNA having a stem-loop structure, including the first and second regions complementary to one another, i.e., sense and antisense strands. The degree of complementarity and orientation of the region is sufficient such that base pairing occurs between the regions, the first and second regions being joined by a loop region, the loop results from a lack of base pairing between nucleotides (or nucleotide analogs) within the loop region. The loop region of an shRNA is a single-stranded region intervening between the sense and antisense strands and can also be referred to as “intervening single-strand”.

As use herein, the term “siD/R-NA” refers to a double-stranded molecule which is composed of both RNA and DNA, and includes hybrids and chimeras of RNA and DNA and prevents translation of a target mRNA. Herein, a hybrid indicates a molecule wherein an oligonucleotide composed of DNA and an oligonucleotide composed of RNA hybridize to each other to form the double-stranded molecule; whereas a chimera indicates that one or both of the strands composing the double stranded molecule can contain RNA and DNA. Standard techniques of introducing siD/R-NA into the cell are used. The siD/R-NA includes a sense nucleic acid sequence of TBC1D7 gene (also referred to as “sense strand”), an antisense nucleic acid sequence of TBC1D7 gene (also referred to as “antisense strand”) or both. The siD/R-NA can be constructed such that a single transcript has both the sense and complementary antisense nucleic acid sequences from the target gene, e.g., a hairpin. The siD/R-NA can either be a dsD/R-NA or shD/R-NA.

As used herein, the term “dsD/R-NA” refers to a construct of two molecules including complementary sequences to one another and that have annealed together via the complementary sequences to form a double-stranded polynucleotide molecule. The nucleotide sequence of two strands can include not only the “sense” or “antisense” polynucleotides sequence selected from a protein coding sequence of target gene sequence, but also polynucleotide having a nucleotide sequence selected from non-coding region of the target gene. One or both of the two molecules constructing the dsD/R-NA are composed of both RNA and DNA (chimeric molecule), or alternatively, one of the molecules is composed of RNA and the other is composed of DNA (hybrid double-strand).

The term “shD/R-NA”, as used herein, refers to an siD/R-NA having a stem-loop structure, including the first and second regions complementary to one another, i.e., sense and antisense strands. The degree of complementarity and orientation of the regions is sufficient such that base pairing occurs between the regions, the first and second regions is joined by a loop region, the loop resulting from a lack of base pairing between nucleotides (or nucleotide analogs) within the loop region. The loop region of an shD/R-NA is a single-stranded region intervening between the sense and antisense strands and can also be referred to as “intervening single-strand”.

(i) Target Sequence

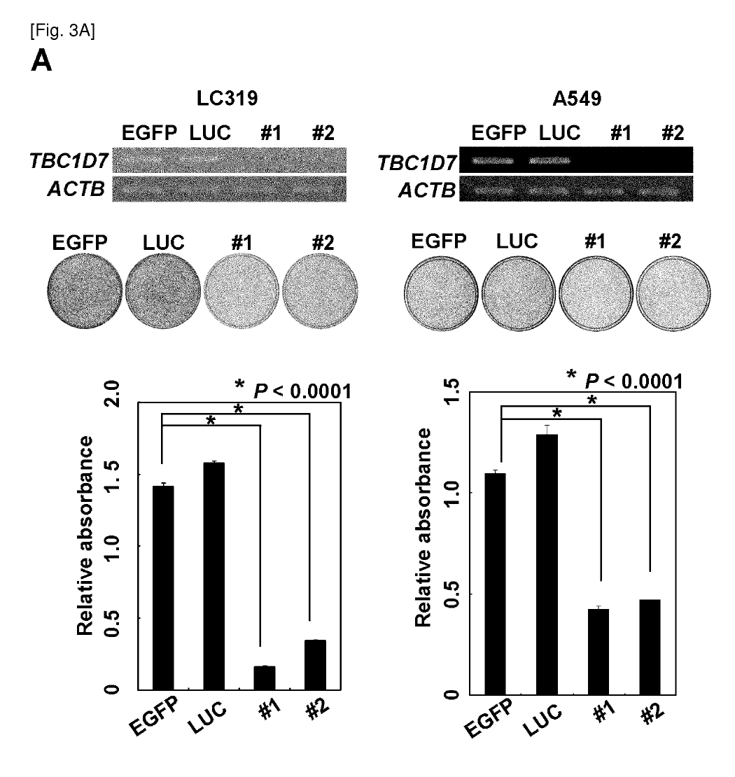

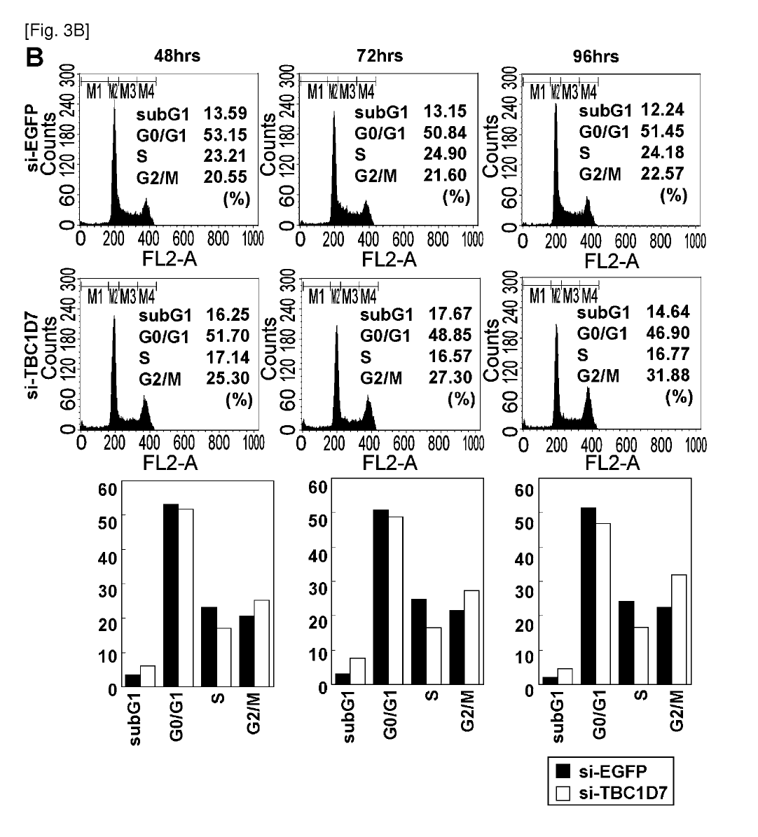

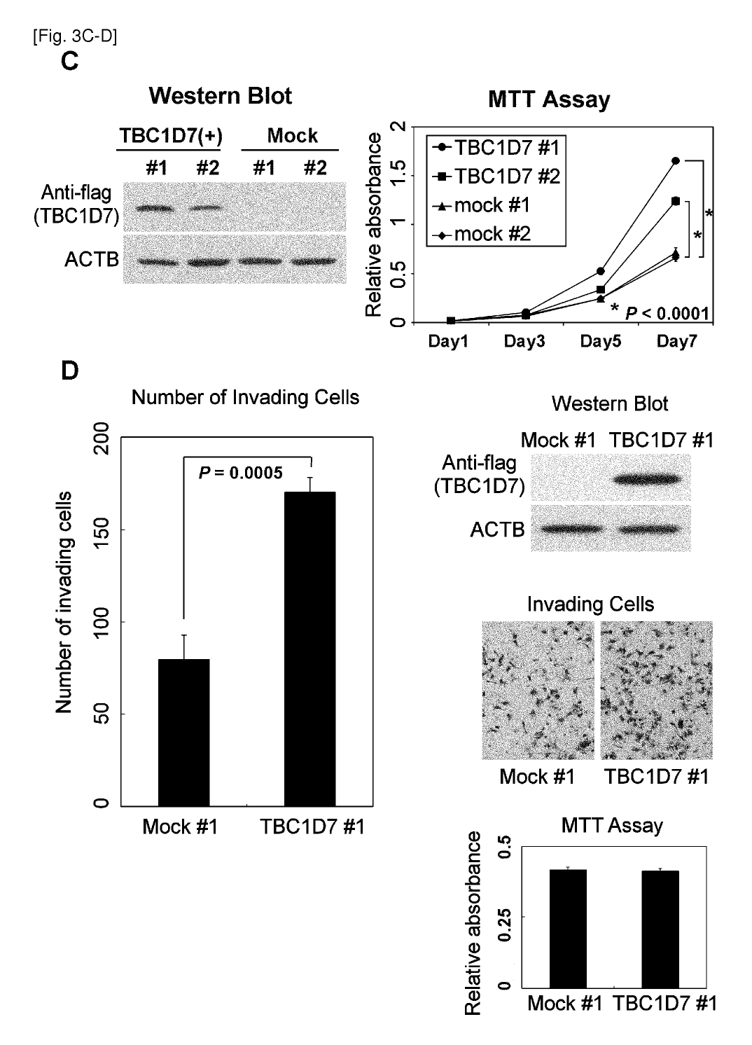

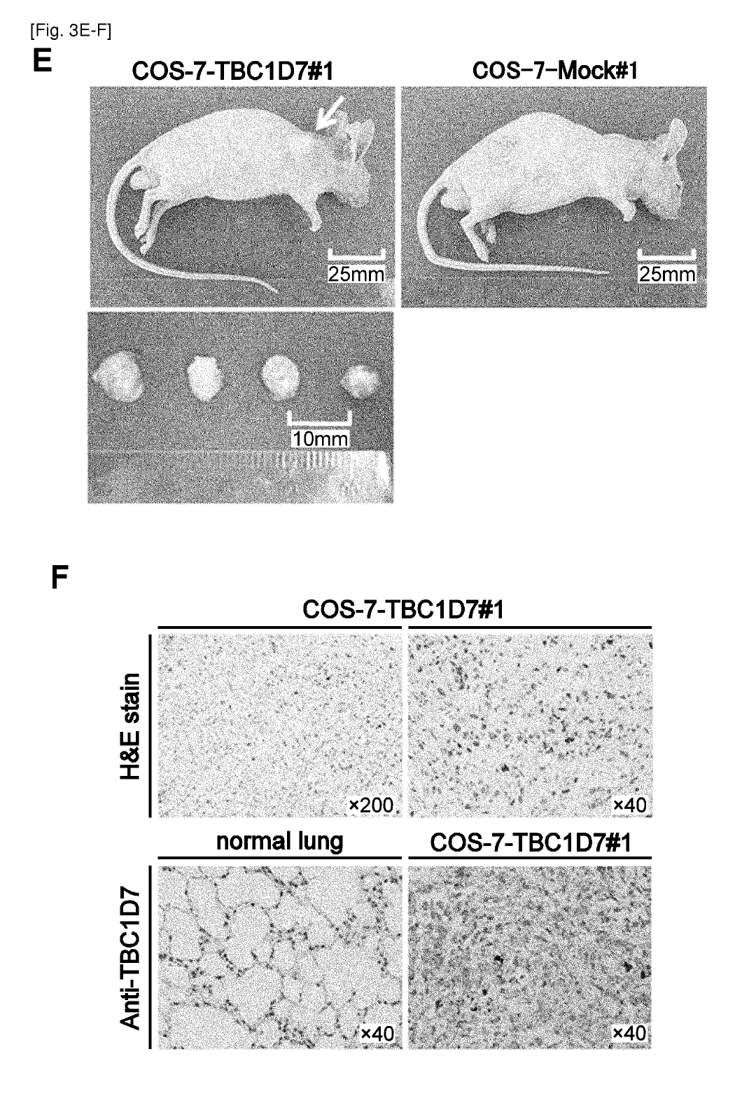

A double-stranded molecule against TBC1D7 gene, which molecule hybridizes to target mRNA, inhibits or reduces production of TBC1D7 protein encoded by TBC1D7 gene by associating with the normally single-stranded mRNA transcript of the gene, thereby interfering with translation and thus, inhibiting expression of the protein encoded by target gene. The expression of TBC1D7 in cancers cell lines was inhibited by two double-stranded molecules of the present invention (FIG. 3A and B).

Therefore the present invention provides isolated double-stranded molecules having the ability to inhibit or reduce the expression of TBC1D7 gene in cancer cells when introduced into a cell. The target sequence of double-stranded molecule is designed by siRNA design algorithm mentioned below.

TBC1D7 target sequence includes, for example, nucleotides

| | |
|---|---|
| 5′-GAACAGTGCAGAGAAGATA-3′ | (SEQ ID NO: 18) |
| or | |
| 5′-GATAAAGTTGTGAGTGGAT-3′ | (SEQ ID NO: 19) |

Specifically, the present invention provides the following double-stranded molecules [1] to [19]:
[1] An isolated double-stranded molecule, which, when introduced into a cell, inhibits in vivo expression of an TBC1D7 gene and cell proliferation, wherein said double-stranded molecule acts at mRNA which matches a target sequence selected from the group of SEQ ID NO: 18 and SEQ ID NO: 19;
[2] The double-stranded molecule of [1], which includes a sense strand and an antisense strand complementary thereto, hybridized to each other to form a double strand, wherein said sense strand includes an oligonucleotide corresponding to a sequence selected from the group consisting of SEQ ID NO: 3, SEQ ID NO: 4, SEQ ID NO: 18 and SEQ ID NO: 19;.
[3] The double-stranded molecule of [1], wherein said target sequence includes at least about 10 contiguous nucleotide from the nucleotide sequence selected from SEQ ID NO: 1.
[4] The double-stranded molecule of [3], wherein said target sequence includes from about 19 to about 25 contiguous nucleotides from the nucleotide sequence selected from SEQ ID NO: 1.
[5] The double-stranded molecule of [2], wherein the sense strand hybridize with antisense strand at the target sequence to form the double-stranded molecule having a length of less than about 100 nucleotides.
[6] The double-stranded molecule of [5], wherein the sense strand hybridize with antisense strand at the target sequence to form the double-stranded molecule having a length of less than about 75 nucleotides.
[7] The double-stranded molecule of [6], wherein the sense strand hybridize with antisense strand at the target sequence to form the double-stranded molecule having a length of less than about 50 nucleotides.
[8] The double-stranded molecule of [7] wherein the sense strand hybridize with antisense strand at the target sequence to form the double-stranded molecule having a length of less than about 25 nucleotides.
[9] The double-stranded molecule of [8], wherein the sense strand hybridize with antisense strand at the target sequence to form the double-stranded molecule having a length of between about 19 and about 25 nucleotides.
[10] The double-stranded molecule of [1], which consists of a single oligonucleotide including both the sense and antisense strands linked by an intervening single-strand.
[11] The double-stranded molecule of [10], which has a general formula 5′-[A]-[B]-[A′]-3′, wherein
[A] is the sense strand including an oligonucleotide corresponding to a sequence selected from the group consisting of SEQ ID NO: 3, SEQ ID NO: 4, SEQ ID NO: 18 and SEQ ID NO: 19 for TBC1D7;
[B] is the intervening single-strand; and
[A′] is the antisense strand including an oligonucleotide corresponding to a sequence complementary to the sequence selected in [A].
[12] The double-stranded molecule of [1], which includes RNA.
[13] The double-stranded molecule of [1], which includes both DNA and RNA.
[14] The double-stranded molecule of [13], which is a hybrid of a DNA polynucleotide and an RNA polynucleotide.
[15] The double-stranded molecule of [14] wherein the sense and the antisense strands are made of DNA and RNA, respectively.
[16] The double-stranded molecule of [13], which is a chimera of DNA and RNA.
[17] The double-stranded molecule of [16], wherein a 5′-end region of the target sequence in the sense strand, and/or a 3′-end region of the complementary sequence of the target sequence in the antisense strand consists of RNA.
[18] The double-stranded molecule of [17], wherein the RNA region consists of 9 to 13 nucleotides; and
[19] The double-stranded molecule of [2], which contains 3′ overhang.

The double-stranded molecule of the present invention will be described in more detail below.

Methods for designing double-stranded molecules having the ability to inhibit target gene expression in cells are known. (See, for example, U.S. Pat. No. 6,506,559, herein incorporated by reference in its entirety). For example, a computer program for designing siRNAs is available from the Ambion website (on the worldwide web at ambion.com/techlib/misc/siRNA_finder.html). The computer program selects target nucleotide sequences for double-stranded molecules based on the following protocol.

Design of Target Sites

1. Beginning with the AUG start codon of the transcript, scan downstream for AA dinucleotide sequences. Record the occurrence of each AA and the 3′ adjacent 19 nucleotides as potential siRNA target sites. Tuschl et al. recommend to avoid designing siRNA to the 5′ and 3′ untranslated regions (UTRs) and regions near the start codon (within 75 bases) as these can be richer in regulatory protein binding sites, and UTR-binding proteins and/or translation initiation complexes can interfere with binding of the siRNA endonuclease complex.

2. Compare the potential target sites to the appropriate genome database (human, mouse, rat, etc.) and eliminate from consideration any target sequences with significant homology to other coding sequences. Basically, BLAST, which can be found on the NCBI server at: on the worldwide web at ncbi.nlm.nih.gov/BLAST/, is used (Altschul S F, et al., Nucleic Acids Res. 1997 Sep. 1; 25(17):3389-402).

3. Select qualifying target sequences for synthesis. Selecting several target sequences along the length of the gene to evaluate is typical.

By the protocol, the target sequence of the isolated double-stranded molecules of the present invention were designed as

TBC1D7 target sequence includes, for example, nucleotides

| | |
|---|---|
| 5′-GAACAGTGCAGAGAAGATA-3′ | (SEQ ID NO: 18) |
| or | |
| 5′-GATAAAGTTGTGAGTGGAT-3′ | (SEQ ID NO: 19) |

Specifically, the present invention provides the following double-stranded molecules targeting the above-mentioned target sequences were respectively examined for their ability to inhibit or reduce the growth of cells expressing the target genes. The growth of TBC1D7 expressing cancer cells, e.g. lung cancer cell lines A549 and LC319, was inhibited by two double stranded molecules of the invention (FIGS. 3A and B). For example, the present invention provides double-stranded molecules targeting any of the sequences selected from the group of

TBC1D7 target sequence includes, for example, nucleotides

| | |
|---|---|
| 5′-GAACAGTGCAGAGAAGATA-3′ | (SEQ ID NO: 18) |
| or | |
| 5′-GATAAAGTTGTGAGTGGAT-3′ | (SEQ ID NO: 19) |

The double-stranded molecules of the present invention are directed to a single target

TBC1D7 gene sequence or can be directed to a plurality of target TBC1D7 gene sequences.

A double-stranded molecule of the present invention targeting the above-mentioned targeting sequence of TBC1D7 gene include isolated polynucleotide(s) that includes any of the nucleic acid sequences of target sequences and/or complementary sequences to the target sequences. Examples of a double-stranded molecule targeting TBC1D7 gene includes an oligonucleotide including the sequence corresponding to SEQ ID NO: 18 or SEQ ID NO: 19, and complementary sequences thereto. However, the present invention is not limited to these examples, and minor modifications in the afore-mentioned nucleic acid sequences are acceptable so long as the modified molecule retains the ability to suppress the expression of TBC1D7 gene. Herein, “minor modification” in a nucleic acid sequence indicates one, two or several substitution, deletion, addition or insertion of nucleic acids to the sequence.

According to the present invention, a double-stranded molecule of the present invention can be tested for its ability using the methods utilized in the Examples (see, (i) RNA interference assay in [EXAMPLE 1]). In the Examples, the double-stranded molecules including sense strands and antisense strands complementary thereto of various portions of mRNA of TBC1D7 genes were tested in vitro for their ability to decrease production of TBC1D7 gene product in cancers cell lines (e.g., using LC319 and A549) according to standard methods. Furthermore, for example, reduction in TBC1D7 gene product in cells contacted with the candidate double-stranded molecule compared to cells cultured in the absence of the candidate molecule can be detected by, e.g. RT-PCR using primers for TBC1D7 gene mRNA mentioned (see,(b)) Semi-quantitative RT-PCR in [EXAMPLE 1]). Sequences which decrease the production of TBC1D7 gene product in vitro cell-based assays can then be tested for there inhibitory effects on cell growth. Sequences which inhibit cell growth in vitro cell-based assay can then be tested for their in vivo ability using animals with cancer, e.g. nude mouse xenograft models, to confirm decreased production of TBC1D7 gene product and decreased cancer cell growth.

When the isolated polynucleotide is RNA or derivatives thereof, base “t” should be replaced with “u” in the nucleotide sequences. As used herein, the term “complementary” refers to Watson-Crick or Hoogsteen base pairing between nucleotides units of a polynucleotide, and the term “binding” means the physical or chemical interaction between two polynucleotides. When the polynucleotide includes modified nucleotides and/or non-phosphodiester linkages, these polynucleotides can also bind each other as same manner. Generally, complementary polynucleotide sequences hybridize under appropriate conditions to form stable duplexes containing few or no mismatches. Furthermore, the sense strand and antisense strand of the isolated polynucleotide of the present invention can form double-stranded molecule or hairpin loop structure by the hybridization. In one embodiment, such duplexes contain no more than 1 mismatch for every 10 matches. In some embodiments, where the strands of the duplex are fully complementary, such duplexes contain no mismatches.

The polynucleotide is the polynucleotide is less than 500, 200, 100, 75, 50, or 25 nucleotides in length for all of the genes. The isolated polynucleotides of the present invention are useful for forming double-stranded molecules against TBC1D7 gene or preparing template DNAs encoding the double-stranded molecules. When the polynucleotides are used for forming double-stranded molecules, the sense strand of polynucleotide can be longer than 19 nucleotides, for example, longer than 21 nucleotides, for example, between about 19 and 25 nucleotides. Accordingly, the present invention provides the double-stranded molecules comprising a sense strand and an antisense strand, wherein the sense strand comprises a nucleotide sequence corresponding to a target sequence. In preferable embodiments, the sense strand hybridizes with antisense strand at the target sequence to form the double-stranded molecule having between 19 and 25 nucleotide pair in length.

The double-stranded molecules of the invention can contain one or more modified nucleotides and/or non-phosphodiester linkages. Chemical modifications well known in the art are capable of increasing stability, availability, and/or cell uptake of the double-stranded molecule. The skilled person will be aware of other types of chemical modification which can be incorporated into the present molecules (WO03/070744; WO2005/045037). In one embodiment, modifications can be used to provide improved resistance to degradation or improved uptake. Examples of such modifications include phosphorothioate linkages, 2′-O-methyl ribonucleotides (especially on the sense strand of a double-stranded molecule), 2′-deoxy-fluoro ribonucleotides, 2′-deoxy ribonucleotides, “universal base” nucleotides, 5′-C-methyl nucleotides, and inverted deoxyabasic residue incorporation (US Pat Appl. No. 20060122137).

In another embodiment, modifications can be used to enhance the stability or to increase targeting efficiency of the double-stranded molecule. Modifications include chemical cross linking between the two complementary strands of a double-stranded molecule, chemical modification of a 3′ or 5′ terminus of a strand of a double-stranded molecule, sugar modifications, nucleobase modifications and/or backbone modifications, 2 -fluoro modified ribonucleotides and 2′-deoxy ribonucleotides (WO2004/029212).

In another embodiment, modifications can be used to increased or decreased affinity for the complementary nucleotides in the target mRNA and/or in the complementary double-stranded molecule strand (WO2005/044976). For example, an unmodified pyrimidine nucleotide can be substituted for a 2-thio, 5-alkynyl, 5-methyl, or 5-propynyl pyrimidine. Additionally, an unmodified purine can be substituted with a 7-deaza, 7-alkyl, or 7-alkenyl purine. In another embodiment, when the double-stranded molecule is a double-stranded molecule with a 3′ overhang, the 3′-terminal nucleotide overhanging nucleotides can be replaced by deoxyribonucleotides (Elbashir S M et al., Genes Dev 2001 Jan. 15, 15(2): 188-200). For further details, published documents for example, US Pat Appl. No. 20060234970 are available. The present invention is not limited to these examples and any known chemical modifications can be employed for the double-stranded molecules of the present invention so long as the resulting molecule retains the ability to inhibit the expression of the target gene.

Furthermore, the double-stranded molecules of the invention can include both DNA and RNA, e.g., dsD/R-NA or shD/R-NA. Specifically, a hybrid polynucleotide of a DNA strand and an RNA strand or a DNA-RNA chimera polynucleotide shows increased stability. Mixing of DNA and RNA, i.e., a hybrid type double-stranded molecule made of a DNA strand (polynucleotide) and an RNA strand (polynucleotide), a chimera type double-stranded molecule including both DNA and RNA on any or both of the single strands (polynucleotides), or the like can be formed for enhancing stability of the double-stranded molecule. The hybrid of a DNA strand and an RNA strand can be either where the sense strand is DNA and the antisense strand is RNA, or the opposite so long as it has an activity to inhibit expression of the target gene when introduced into a cell expressing the gene.

In some embodiments, the sense strand polynucleotide is DNA and the antisense strand polynucleotide is RNA. Also, the chimera type double-stranded molecule can be either where both of the sense and antisense strands are composed of DNA and RNA, or where any one of the sense and antisense strands is composed of DNA and RNA so long as it has an activity to inhibit expression of the target gene when introduced into a cell expressing the gene. In order to enhance stability of the double-stranded molecule, in some embodiments, the molecule contains as much DNA as possible, whereas to induce inhibition of the target gene expression, the molecule is required to be RNA within a range to induce sufficient inhibition of the expression. In one example of the chimera type double-stranded molecule, an upstream partial region (i.e., a region flanking to the target sequence or complementary sequence thereof within the sense or antisense strands) of the double-stranded molecule is RNA.

In some embodiments, the upstream partial region indicates the 5′ side (5′-end) of the sense strand and the 3′ side (3′-end) of the antisense strand. That is, in some embodiments, a region flanking to the 3′-end of the antisense strand, or both of a region flanking to the 5′-end of sense strand and a region flanking to the 3′-end of antisense strand consists of RNA. For instance, the chimera or hybrid type double-stranded molecule of the present invention include following combinations.

sense strand:

5′-[- - - DNA - - - ]-3′

3′-(RNA)-[DNA]-5′

: antisense strand,

sense strand:

5′-(RNA)-[DNA]-3′

3′-(RNA)-[DNA]-5′

: antisense strand, and

sense strand:

5′-(RNA)-[DNA]-3′

3′-( - - - RNA - - - )-5′

: antisense strand.

The upstream partial region can be a domain of about 9 to 13 nucleotides counted from the terminus of the target sequence or complementary sequence thereto within the sense or antisense strands of the double-stranded molecules. Moreover, examples of such chimera type double-stranded molecules include those having a strand length of 19 to 21 nucleotides in which at least the upstream half region (5′ side region for the sense strand and 3′ side region for the antisense strand) of the polynucleotide is RNA and the other half is DNA. In such a chimera type double-stranded molecule, the effect to inhibit expression of the target gene is much higher when the entire antisense strand is RNA (US Pat Appl. No. 20050004064).

In the present invention, the double-stranded molecule can form a hairpin, for example, a short hairpin RNA (shRNA) and short hairpin made of DNA and RNA (shD/R-NA). The shRNA or shD/R-NA is a sequence of RNA or mixture of RNA and DNA making a tight hairpin turn that can be used to silence gene expression via RNA interference. The shRNA or shD/R-NA includes the sense target sequence and the antisense target sequence on a single strand wherein the sequences are separated by a loop sequence. Generally, the hairpin structure is cleaved by the cellular machinery into dsRNA or dsD/R-NA, which is then bound to the RNA-induced silencing complex (RISC). This complex binds to and cleaves mRNAs which match the target sequence of the dsRNA or dsD/R-NA.

A loop sequence made of an arbitrary nucleotide sequence can be located between the sense and antisense sequence in order to form the hairpin loop structure. Thus, the present invention also provides a double-stranded molecule having the general formula 5′-[A]-[B]-[A′]-3′, wherein [A] is the sense strand including a target sequence, [B] is an intervening single-strand and [A′] is the antisense strand including a complementary sequence to [A]. The target sequence can be selected from the group consisting of, for example, SEQ ID NO: 18 or SEQ ID NO: 19 nucleotides

The present invention is not limited to these examples, and the target sequence in [A] can be modified sequences from these examples so long as the double-stranded molecule retains the ability to suppress the expression of the targeted TBC1D7 gene and result in inhibits or reduces the cell expressing these genes. The region [A] hybridizes to [A′] to form a loop including the region [B]. The intervening single-stranded portion [B], i.e., the loop sequence can be 3 to 23 nucleotides in length. The loop sequence, for example, can be selected from group consisting of following sequences (on the worldwide web at ambion.com/techlib/tb/tb_{—}506.html). Furthermore, loop sequence consisting of 23 nucleotides also provides active siRNA (Jacque J M et al., Nature 2002 Jul. 25, 418(6896): 435-8, Epub 2002 Jun. 26):

CCC, CCACC, or CCACACC: Jacque J M et al., Nature 2002 Jul. 25, 418(6896): 435-8, Epub 2002 Jun. 26;

UUCG: Lee N S et al., Nat Biotechnol 2002 May, 20(5): 500-5; Fruscoloni P et al., Proc Natl Acad Sci USA 2003 Feb. 18, 100(4): 1639-44, Epub 2003 Feb. 10; and

UUCAAGAGA: Dykxhoorn D M et al., Nat Rev Mol Cell Biol 2003 June, 4(6): 457-67.

Exemplary double-stranded molecules having hairpin loop structure of the present invention are shown below. In the following structure, the loop sequence can be selected from group consisting of AUG, CCC, UUCG, CCACC, CTCGAG, AAGCUU, CCACACC, and UUCAAGAGA; however, the present invention is not limited thereto:

GAACAGUGCAGAGAAGAUAUU-[B]-UAUCUUCUCUGCACUGUUC (for target sequence SEQ ID NO: 18); and

GAUAAAGUUGUGAGUGGAUUU-[B]-AUCCACUCACAACUUUAUC (for target sequence SEQ ID NO: 19).

Furthermore, in order to enhance the inhibition activity of the double-stranded molecules, nucleotide “u” can be added to 3′end of the antisense strand of the target sequence, as 3′ overhangs. The number of “u”s to be added is at least 2, generally 2 to 10, for example, 2 to 5. The added “u”s form single strand at the 3′end of the antisense strand of the double-stranded molecule.

The method of preparing the double-stranded molecule can use any chemical synthetic method known in the art. According to the chemical synthesis method, sense and antisense single-stranded polynucleotides are separately synthesized and then annealed together via an appropriate method to obtain a double-stranded molecule. In one embodiment for the annealing, the synthesized single-stranded polynucleotides are mixed in a molar ratio of at least about 3:7, for example, about 4:6, for example, substantially equimolar amount (i.e., a molar ratio of about 5:5). Next, the mixture is heated to a temperature at which double-stranded molecules dissociate and then is gradually cooled down. The annealed double-stranded polynucleotide can be purified by usually employed methods known in the art. Example of purification methods include methods utilizing agarose gel electrophoresis or wherein remaining single-stranded polynucleotides are optionally removed by, e.g., degradation with appropriate enzyme.

The regulatory sequences flanking target sequences can be identical or different, such that their expression can be modulated independently, or in a temporal or spatial manner. The double-stranded molecules can be transcribed intracellularly by cloning TBC1D7 gene templates into a vector containing, e.g., a RNA pol III transcription unit from the small nuclear RNA (snRNA) U6 or the human H1 RNA promoter.

(ii) Vector

Also included in the invention is a vector containing one or more of the double-stranded molecules described herein, and a cell containing the vector. A vector of the present invention encodes a double-stranded molecule of the present invention in an expressible form. Herein, the phrase “in an expressible form” indicates that the vector, when introduced into a cell, will express the molecule. In one embodiment, the vector includes regulatory elements necessary for expression of the double-stranded molecule. Such vectors of the present invention can be used for producing the present double-stranded molecules, or directly as an active ingredient for treating cancer.

Vectors of the present invention can be produced, for example, by cloning a sequence including target sequence into an expression vector so that regulatory sequences are operatively-linked to the sequence in a manner to allow expression (by transcription of the DNA molecule) of both strands (Lee N S et al., Nat Biotechnol 2002 May, 20(5): 500-5). For example, RNA molecule that is the antisense to mRNA is transcribed by a first promoter (e.g., a promoter sequence flanking to the 3′ end of the cloned DNA) and RNA molecule that is the sense strand to the mRNA is transcribed by a second promoter (e.g., a promoter sequence flanking to the 5′ end of the cloned DNA). The sense and antisense strands hybridize in vivo to generate a double-stranded molecule constructs for silencing of the gene. Alternatively, two vectors constructs respectively encoding the sense and antisense strands of the double-stranded molecule are utilized to respectively express the sense and anti-sense strands and then forming a double-stranded molecule construct. Furthermore, the cloned sequence can encode a construct having a secondary structure (e.g., hairpin); namely, a single transcript of a vector contains both the sense and complementary antisense sequences of the target gene.

The vectors of the present invention can also be equipped so to achieve stable insertion into the genome of the target cell (see, e.g., Thomas K R & Capecchi M R, Cell 1987, 51: 503-12 for a description of homologous recombination cassette vectors). See, e.g., Wolff et al., Science 1990, 247: 1465-8; U.S. Pat. Nos. 5,580,859; 5,589,466; 5,804,566; 5,739,118; 5,736,524; 5,679,647; and WO 98/04720. Examples of DNA-based delivery technologies include “naked DNA”, facilitated (bupivicaine, polymers, peptide-mediated) delivery, cationic lipid complexes, and particle-mediated (“gene gun”) or pressure-mediated delivery (see, e.g., U.S. Pat. No. 5,922,687).

The vectors of the present invention can be, for example, viral or bacterial vectors.

Examples of expression vectors include attenuated viral hosts, for example, vaccinia or fowlpox (see, e.g., U.S. Pat. No. 4,722,848). This approach involves the use of vaccinia virus, e.g., as a vector to express nucleotide sequences that encode the double-stranded molecule. Upon introduction into a cell expressing the target gene, the recombinant vaccinia virus expresses the molecule and thereby suppresses the proliferation of the cell. Another example of useable vector includes Bacille Calmette Guerin (BCG). BCG vectors are described in Stover et al., Nature 1991, 351: 456-60. A wide variety of other vectors are useful for therapeutic administration and production of the double-stranded molecules; examples include adeno and adeno-associated virus vectors, retroviral vectors, Salmonella typhi vectors, detoxified anthrax toxin vectors, and the like. See, e.g., Shata et al., Mol Med Today 2000, 6: 66-71; Shedlock et al., J Leukoc Biol 2000, 68: 793-806; and Hipp et al., In Vivo 2000, 14: 571-85.

(iii) Methods of Inhibiting or Reducing a Growth of Cancer Cells and Treating or Preventing Cancer Using Double-Stranded Molecules

In the present invention, double-stranded molecules targeting the above-mentioned target sequences were examined for their ability to inhibit or reduce the growth of cells (over)expressing the target genes. The growth of cancer cells (over)expressing TBC1D7 gene, was inhibited or reduced by double-stranded molecules of the present invention; the growth of the cell (over)expressing TBC1D7 gene was inhibited or reduced by the double-stranded molecules of the present invention; the growth of the TBC1D7 (over)expressing cells, e.g. lung cancer cell line A549 and LC319, was inhibited by two double stranded molecules (FIGS. 3A and B).

Therefore, the present invention provides methods for inhibiting cell growth, i.e., cancerous cell growth of a cell from a cancer resulting from overexpression of a TBC1D7 gene, or that is mediated by a TBC1D7 gene, by inhibiting the expression of the TBC1D7 gene. TBC1D7 gene expression can be inhibited by any of the aforementioned double-stranded molecules of the present invention which specifically target expression of a complementary TBC1D7 gene or the vectors of the present invention that can express any of the double-stranded molecules. Such ability of the present double-stranded molecules and vectors to inhibit cell growth of cancerous cells indicates that they can be used for methods for treating a cancer resulting from overexpression of a TBC1D7 gene, or that is mediated by a TBC1D7 gene. Thus, the present invention provides methods to treat patients with a cancer resulting from overexpression of a TBC1D7 gene, or that is mediated by a TBC1D7 gene by administering a double-stranded molecule, i.e., an inhibitory nucleic acid, against a TBC1D7 gene or a vector expressing the molecule without adverse effect because those genes were hardly detected in normal organs.

Specifically, the present invention provides the following methods [1] to [22]:

[1] A method for inhibiting or reducing a growth of a cell (over)expressing a TBC1D7 gene or a method for treating or preventing cancer (over)expressing TBC1D7 gene, wherein said method including the step of giving at least one double-stranded molecule, wherein said double-stranded molecule is introduced into a cell, and inhibits or reduces in vivo expression of said TBC1D7 gene.

[2] The method of [1], wherein said double-stranded molecule acts at mRNA which shares sequence identity with or is complementary to a target sequence selected from the group of SEQ ID NO: 18 and SEQ ID NO: 19.

[3] The method of [2], wherein said double-stranded molecule includes a sense strand and an antisense strand complementary thereto, hybridized to each other to form a double strand, wherein said sense strand includes an oligonucleotide corresponding to a sequence selected from the group consisting of SEQ ID NO: 3, SEQ ID NO: 4, SEQ ID NO: 18 and SEQ ID NO: 19.

[4] The method of [1], wherein a plurality of double-stranded molecules are administered; In some embodiments, the double-stranded molecules include different nucleic acid sequences.

[5] The method of [4], wherein the plurality of double-stranded molecules target the same gene;

[6] The method of [1], wherein the double-stranded molecule has a length of less than about 100 nucleotides;

[7] The method of [6], wherein the double-stranded molecule, wherein the sense strand of the double-stranded molecule hybridizes with antisense strand at the target sequence to form the double-stranded molecule having a length of less than about 75 nucleotides;

[8] The method of [7], wherein the double-stranded molecule, wherein the sense strand of the double-stranded molecule hybridizes with antisense strand at the target sequence to form the double-stranded molecule having a length of less than about 50 nucleotides;

[9] The method of [8], wherein the double-stranded molecule, wherein the sense strand of the double-stranded molecule hybridizes with antisense strand at the target sequence to form the double-stranded molecule having a length of less than about 25 nucleotides;

[10] The method of [9], wherein the double-stranded molecule, wherein the sense strand of the double-stranded molecule hybridizes with antisense strand at the target sequence to form the double-stranded molecule having a length of between about 19 and about 25 nucleotides in length;

[11] The method of [1], wherein said double-stranded molecule consists of a single oligonucleotide including both the sense and antisense strands linked by an intervening single-strand.

[12] The method of [11], wherein said double-stranded molecule has a general formula 5′-[A]-[B]-[A′]-3′, wherein

[A] is the sense strand including an oligonucleotide corresponding to a sequence selected from the group consisting of SEQ ID NO: 3, SEQ ID NO: 4, SEQ ID NO: 18 and SEQ ID NO: 19;

[B] is the intervening single-strand; and

[A′] is the antisense strand including an oligonucleotide corresponding to a sequence complementary to the sequence selected in [A].

[13] The method of [1], wherein the double-stranded molecule includes RNA.

[14] The method of [1], wherein the double-stranded molecule includes both DNA and RNA.

[15] The method of [14], wherein the double-stranded molecule is a hybrid of a DNA polynucleotide and an RNA polynucleotide.

[16] The method of [15] wherein the sense and antisense strand polynucleotides a made of DNA and RNA, respectively.

[17] The method of [14], wherein the double-stranded molecule is a chimera of DNA and RNA.

[18] The method of [17], wherein a region flanking to the 5′-end of one or both of the sense and antisense polynucleotides a made of RNA.

[19] The method of [18], wherein the flanking region consists of 9 to 13 nucleotides.

[20] The method of [1], wherein the double-stranded molecule contains 3′ overhangs.

[21] The method of [1], wherein the double-stranded molecule is encoded by a vector.

[22] The method of [21], wherein said double-stranded molecule has a general formula 5′-[A]-[B]-[A′]-3′, wherein

[A] is the sense strand including an oligonucleotide corresponding to a sequence selected from the group consisting of SEQ ID NO: 3, SEQ ID NO: 4, SEQ ID NO: 18 and SEQ ID NO: 19;

[B] is the intervening single-strand; and

[A′] is the antisense strand including an oligonucleotide corresponding to a sequence complementary to the sequence selected in [A].

[23] The method of [1], wherein the double-stranded molecule is contained in a composition which includes in addition to the molecule a transfection-enhancing agent and cell permeable agent.

The method of the present invention will be described in more detail below.

The growth of cells (over)expressing a TBC1D7 gene is inhibited by contacting the cells with a double-stranded molecule against TBC1D7 gene, a vector expressing the molecule or a composition including the same. The cell is further contacted with a transfection agent. Suitable transfection agents are known in the art. The phrase “inhibition of cell growth” indicates that the cell proliferates at a lower rate or has decreased viability compared to a cell not exposed to the molecule. Cell growth can be measured by methods known in the art, e.g., using the MTT cell proliferation assay.

The growth of any kind of cell can be suppressed according to the present method so long as the cell expresses or over-expresses the target gene of the double-stranded molecule of the present invention. Exemplary cells include cancers cells. Thus, patients suffering from or at risk of developing disease related to TBC1D7 gene can be treated by administering at least one of the present double-stranded molecules, at least one vector expressing at least one of the molecules or at least one composition including at least one of the molecules. For example, patients of cancers can be treated according to the present methods. The type of cancer can be identified by standard methods according to the particular type of tumor to be diagnosed. In some embodiments, patients treated by the methods of the present invention are selected by detecting the (over)expression of a TBC1D7 gene in a biopsy from the patient by RT-PCR, hybridization or immunoassay. In some embodiments, before the treatment of the present invention, the biopsy specimen from the subject is confirmed for TBC1D7 gene over-expression by methods known in the art, for example, immunohistochemical analysis, hybridization or RT-PCR (see, (b) Semi-quantitative RT-PCR, (c) Northern-blot analysis, (e) Western-blotting or (f) Immunohistochemistry in [EXAMPLE 1]).

According to the present method to inhibit or reduce cell growth and thereby treatcancer, when administering a plurality of double-stranded molecules of the invention (or vectors expressing or compositions containing the same), each of the molecules can be directed to a different target sequence of the same gene, or a different target sequence of different genes. For example, the method can utilize different double-stranded molecules directing to the same TBC1D7 gene transcript. Alternatively, for example, the method can utilize double-stranded molecules directed to one, two or more target sequences selected from same TBC1D7 gene.

For inhibiting cell growth, a double-stranded molecule of present invention can be directly introduced into the cells in a form to achieve binding of the molecule with corresponding mRNA transcripts. Alternatively, as described above, a DNA encoding the double-stranded molecule can be introduced into cells as a vector. For introducing the double-stranded molecules and vectors into the cells, transfection-enhancing agent, for example, FuGENE (Roche diagnostics), Lipofectamine 2000 (Invitrogen), Oligofectamine (Invitrogen), and Nucleofector (Wako pure Chemical), can be employed.

A treatment is determined efficacious if it leads to clinical benefit for example, reduction in expression of the TBC1D7 gene, or a decrease in size, prevalence, or metastatic potential of the cancer in the subject. When the treatment is applied prophylactically, “efficacious” means that it retards or prevents cancers from forming or prevents or alleviates a clinical symptom of cancer. Efficaciousness is determined in association with any known method for diagnosing or treating the particular tumor type.

It is understood that the double-stranded molecule of the invention degrades the target mRNA (TBC1D7 gene transcript) in substoichiometric amounts. Without wishing to be bound by any theory, it is believed that the double-stranded molecule of the invention causes degradation of the target mRNA in a catalytic manner. Thus, compared to standard cancer therapies, significantly less a double-stranded molecule needs to be delivered at or near the site of cancer to exert therapeutic effect.

One skilled in the art can readily determine an effective amount of the double-stranded molecule of the invention to be administered to a given subject, by taking into account factors for example, body weight, age, sex, type of disease, symptoms and other conditions of the subject; the route of administration; and whether the administration is regional or systemic. Generally, an effective amount of the double-stranded molecule of the invention includes an intercellular concentration at or near the cancer site of from about 1 nanomolar (nM) to about 100 nM, for example, from about 2 nM to about 50 nM, for example, from about 2.5 nM to about 10 nM. It is contemplated that greater or smaller amounts of the double-stranded molecule can be administered.

The present methods can be used to inhibit the growth or metastasis of cancer; for example, a cancer resulting from overexpression of a TBC1D7 gene or that is mediated by a TBC1D7 gene, e.g., lung cancer or esophageal cancer. In particular, a double-stranded molecule directed to a target sequence selected from the group consisting of SEQ ID NO: 18 and SEQ ID NO: 19 for TBC1D7 finds use for the treatment of cancers.

For treating cancer, e.g., a cancer promoted by a TBC1D7 gene, the double-stranded molecule of the invention can also be administered to a subject in combination with a pharmaceutical agent different from the double-stranded molecule. Alternatively, the double-stranded molecule of the invention can be administered to a subject in combination with another therapeutic method designed to treat cancer. For example, the double-stranded molecule of the invention can be administered in combination with therapeutic methods currently employed for treating cancer or preventing cancer metastasis (e.g., radiation therapy, surgery and treatment using chemotherapeutic agents, for example, cisplatin, carboplatin, cyclophosphamide, 5-fluorouracil, adriamycin, daunorubicin or tamoxifen).

In the present methods, the double-stranded molecule can be administered to the subject either as a naked double-stranded molecule, in conjunction with a delivery reagent, or as a recombinant plasmid or viral vector which expresses the double-stranded molecule.

Suitable delivery reagents for administration in conjunction with the present a double-stranded molecule include the Mirus Transit TKO lipophilic reagent; lipofectin; lipofectamine; cellfectin; or polycations (e.g., polylysine), or liposomes. In one embodiment, the delivery reagent is a liposome. Liposomes can aid in the delivery of the double-stranded molecule to a particular tissue, for example, retinal or tumor tissue, and can also increase the blood half-life of the double-stranded molecule. Liposomes suitable for use in the invention are formed from standard vesicle-forming lipids, which generally include neutral or negatively charged phospholipids and a sterol, for example, cholesterol. The selection of lipids is generally guided by consideration of factors for example, the desired liposome size and half-life of the liposomes in the blood stream. A variety of methods are known for preparing liposomes, for example as described in Szoka et al., Ann Rev Biophys Bioeng 1980, 9: 467; and U.S. Pat. Nos. 4,235,871; 4,501,728; 4,837,028; and 5,019,369, the entire disclosures of which are herein incorporated by reference.

In some embodiments, the liposomes encapsulating the present double-stranded molecule includes a ligand molecule that can deliver the liposome to the cancer site. Ligands which bind to receptors prevalent in tumor or vascular endothelial cells, for example, monoclonal antibodies that bind to tumor antigens or endothelial cell surface antigens, find use. In some embodiments, the liposomes encapsulating the present double-stranded molecule are modified so as to avoid clearance by the mononuclear macrophage and reticuloendothelial systems, for example, by having opsonization-inhibition moieties bound to the surface of the structure. In one embodiment, a liposome of the invention can include both opsonization-inhibition moieties and a ligand.

Opsonization-inhibiting moieties for use in preparing the liposomes of the invention are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer which significantly decreases the uptake of the liposomes by the macrophage-monocyte system (“MMS”) and reticuloendothelial system (“RES”); e.g., as described in U.S. Pat. No. 4,920,016, the entire disclosure of which is herein incorporated by reference. Liposomes modified with opsonization-inhibition moieties thus remain in the circulation much longer than unmodified liposomes. For this reason, such liposomes are sometimes called “stealth” liposomes.

Stealth liposomes are known to accumulate in tissues fed by porous or “leaky” microvasculature. Thus, target tissue characterized by such microvasculature defects, for example, solid tumors, will efficiently accumulate these liposomes; see Gabizon et al., Proc Natl Acad Sci USA 1988, 18: 6949-53. In addition, the reduced uptake by the RES lowers the toxicity of stealth liposomes by preventing significant accumulation in liver and spleen. Thus, liposomes of the invention that are modified with opsonization-inhibition moieties can deliver the present double-stranded molecule to tumor cells.

Opsonization inhibiting moieties suitable for modifying liposomes can be water-soluble polymers with a molecular weight from about 500 to about 40,000 daltons, for example, from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers for example, polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, for example, ganglioside GM₁. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups.

In some embodiments, the opsonization-inhibiting moiety is a PEG, PPG, or derivatives thereof. Liposomes modified with PEG or PEG-derivatives are sometimes called “PEGylated liposomes”.

The opsonization inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH₃ and a solvent mixture for example, tetrahydrofuran and water in a 30:12 ratio at 60 degrees C.

Vectors expressing a double-stranded molecule of the invention are discussed above.

Such vectors expressing at least one double-stranded molecule of the invention can also be administered directly or in conjunction with a suitable delivery reagent, including the Mirus Transit LT1 lipophilic reagent; lipofectin; lipofectamine; cellfectin; polycations (e.g., polylysine) or liposomes. Methods for delivering recombinant viral vectors, which express a double-stranded molecule of the invention, to an area of cancer in a patient are within the skill of the art.

The double-stranded molecule of the invention can be administered to the subject by any means suitable for delivering the double-stranded molecule into cancer sites. For example, the double-stranded molecule can be administered by gene gun, electro-poration, or by other suitable parenteral or enteral administration routes.

Suitable enteral administration routes include oral, rectal, or intranasal delivery.

Suitable parenteral administration routes include intravascular administration (e.g., intravenous bolus injection, intravenous infusion, intra-arterial bolus injection, intra-arterial infusion and catheter instillation into the vasculature); peri- and intra-tissue injection (e.g., peri-tumoral and intra-tumoral injection); subcutaneous injection or de-position including subcutaneous infusion (for example, by osmotic pumps); direct application to the area at or near the site of cancer, for example by a catheter or other placement device (e.g., a suppository or an implant including a porous, non-porous, or gelatinous material); and inhalation. In some embodiments, injections or infusions of the double-stranded molecule or vector be given at or near the site of cancer.

The double-stranded molecule of the invention can be administered in a single dose or in multiple doses. Where the administration of the double-stranded molecule of the invention is by infusion, the infusion can be a single sustained dose or can be delivered by multiple infusions. Injection of the agent can be directly into the tissue or near the site of cancer. Multiple injections of the agent into the tissue at or near the site of cancer can be administered.

One skilled in the art can also readily determine an appropriate dosage regimen for administering the double-stranded molecule of the invention to a given subject. For example, the double-stranded molecule can be administered to the subject once, for example, as a single injection or deposition at or near the cancer site. Alternatively, the double-stranded molecule can be administered once or twice daily to a subject for a period of from about three to about twenty-eight days, for example, from about seven to about ten days. In one exemplary dosage regimen, the double-stranded molecule is injected at or near the site of cancer once a day for seven days. Where a dosage regimen includes multiple administrations, it is understood that the effective amount of a double-stranded molecule administered to the subject can include the total amount of a double-stranded molecule administered over the entire dosage regimen.

(iv) Compositions

Furthermore, the present invention provides pharmaceutical compositions including at least one of the present double-stranded molecules or the vectors coding for the molecules. Specifically, the present invention provides the following compositions [1] to [24]:

[1] A composition for inhibiting or reducing a growth of cell expressing TBC1D7 gene, or a composition for treating or preventing a cancer expressing a TBC1D7 gene which including at least one double-stranded molecule, wherein said double-stranded molecule is introduced into a cell, inhibits or reduces in vivo expression of said gene.

[2] The composition of [1], wherein said double-stranded molecule acts at mRNA which matched a target sequence selected from the group SEQ ID NO: 18 and SEQ ID NO: 19 for TBC1D7.

[3] The composition of [2], wherein said double-stranded molecule includes a sense strand and an antisense strand complementary thereto, hybridized to each other to form a double strand, wherein said sense strand includes an oligonucleotide corresponding to a sequence selected from the group consisting of SEQ ID NO: 3, SEQ ID NO: 4, SEQ ID NO: 18 and SEQ ID NO: 19.

The composition of [1], wherein the cancer to be treated is a cancer resulting from overexpression of TBC1D7 gene, or which is mediated by a TBC1D7 gene.

[4] The composition of [1], wherein the cancer to be treated is lung cancer or esophageal cancer;

[5] The composition of [4], wherein the lung cancer is small cell lung cancer or non-small cell lung cancer;

[6] The composition of [1], wherein the composition contains plural kinds of the double-stranded molecules;

[7] The composition of [6], wherein the plural kinds of the double-stranded molecules target the same gene;

[8] The composition of [1], wherein the sense strand of the double-stranded molecule has a length of less than about 100 nucleotides;

[9] The composition of [8], wherein the sense strand of the double-stranded molecule has a length of less than about 75 nucleotides;

[10] The composition of [9], wherein the sense strand of the double-stranded molecule has a length of less than about 50 nucleotides;

[11] The composition of [10], wherein the sense strand of the double-stranded molecule has a length of less than about 25 nucleotides;

[12] The composition of [11], wherein the sense strand of the double-stranded molecule has a length of between about 19 and about 25 nucleotides;

[13] The composition of [1], wherein said double-stranded molecule consists of a single oligonucleotide including both the sense and antisense strands linked by an intervening single-strand.

[14] The composition of [13], wherein said double-stranded molecule has a general formula 5′-[A]-[B]-[A′]-3′, wherein

[A] is the sense strand including an oligonucleotide corresponding to a sequence selected from the group consisting of SEQ ID NO: 3, SEQ ID NO: 4, SEQ ID NO: 18 and SEQ ID NO: 19;

[B] is the intervening single-strand; and

[A′] is the antisense strand including an oligonucleotide corresponding to a sequence complementary to the sequence selected in [A].

[15] The composition of [1], wherein the double-stranded molecule includes RNA;

[16] The composition of [1], wherein the double-stranded molecule includes DNA and RNA;

[17] The composition of [16], wherein the double-stranded molecule is a hybrid of a DNA polynucleotide and an RNA polynucleotide;

[18] The composition of [17], wherein the sense and antisense strand polynucleotides are made of DNA and RNA, respectively;

[19] The composition of [18], wherein the double-stranded molecule is a chimera of DNA and RNA;

[20] The composition of [19], wherein at least a region flanking to the 5′-end of one or both of the sense and antisense polynucleotides consists of RNA.

[21] The composition of [20], wherein the flanking region consists of 9 to 13 nucleotides;

[22] The composition of [1], wherein the double-stranded molecule contains 3′ overhangs;

[23] The composition of [1], wherein the double-stranded molecule is encoded by a vector and contained in the composition;

[24] The composition of [1], which further including a transfection-enhancing agent, cell permeable agent and pharmaceutically acceptable carrier.

The method of the present invention will be described in more detail below.

The double-stranded molecules of the invention can be formulated as pharmaceutical compositions prior to administering to a subject, according to techniques known in the art. Pharmaceutical compositions of the present invention are characterized as being at least sterile and pyrogen-free. As used herein, “pharmaceutical formulations” include formulations for human and veterinary use. Methods for preparing pharmaceutical compositions of the invention are within the skill in the art, for example as described in Remington's Pharmaceutical Science, 17th ed., Mack Publishing Company, Easton, Pa. (1985), the entire disclosure of which is herein incorporated by reference.

The present pharmaceutical formulations include at least one of the double-stranded molecules or vectors encoding them of the present invention (e.g., 0.1 to 90% by weight), or a physiologically acceptable salt of the molecule, mixed with a physiologically acceptable carrier medium. Exemplary physiologically acceptable carrier media include, for example, water, buffered water, normal saline, 0.4% saline, 0.3% glycine, hyaluronic acid and the like.

According to the present invention, the composition can contain plural kinds of the double-stranded molecules, each of the molecules can be directed to the same target sequence, or different target sequences of TBC1D7 gene. For example, the composition can contain double-stranded molecules directed to TBC1D7 gene. Alternatively, for example, the composition can contain double-stranded molecules directed to one, two or more target sequences selected from TBC1D7 gene.

Furthermore, the present composition can contain a vector coding for one or plural double-stranded molecules. For example, the vector can encode one, two or several kinds of the present double-stranded molecules. Alternatively, the present composition can contain plural kinds of vectors, each of the vectors coding for a different double-stranded molecule. Moreover, the present double-stranded molecules can be contained as liposomes in the present composition. See under the item of “Methods of treating cancer” for details of liposomes.

Pharmaceutical compositions of the invention can also include conventional pharmaceutical excipients and/or additives. Suitable pharmaceutical excipients include stabilizers, antioxidants, osmolality adjusting agents, buffers, and pH adjusting agents. Suitable additives include physiologically biocompatible buffers (e.g., tromethamine hydrochloride), additions of chelants (for example, for example, DTPA or DTPA-bisamide) or calcium chelate complexes (for example calcium DTPA, CaNaDTPA-bisamide), or, optionally, additions of calcium or sodium salts (for example, calcium chloride, calcium ascorbate, calcium gluconate or calcium lactate). Pharmaceutical compositions of the invention can be packaged for use in liquid form, or can be lyophilized.

For solid compositions, conventional nontoxic solid carriers can be used; for example, pharmaceutical grades of mannitol, lactose, starch, magnesium stearate, sodium saccharin, talcum, cellulose, glucose, sucrose, magnesium carbonate, and the like.

For example, a solid pharmaceutical composition for oral administration can include any of the carriers and excipients listed above and 10-95%, for example, 25-75%, of one or more double-stranded molecule of the invention. A pharmaceutical composition for aerosol (inhalational) administration can include 0.01-20% by weight, for example, 1-10% by weight, of one or more double-stranded molecule of the invention encapsulated in a liposome as described above, and propellant. A carrier can also be included as desired; e.g., lecithin for intranasal delivery.

In addition to the above, the present composition can contain other pharmaceutical active ingredients so long as they do not inhibit the in vivo function of the present double-stranded molecules. For example, the composition can contain chemotherapeutic agents conventionally used for treating cancers.

The present invention also provides the use of the double-stranded nucleic acid molecules of the present invention in manufacturing a pharmaceutical composition for treating a cancer (over)expressing the TBC1D7 gene. For example, the present invention relates to the use of double-stranded nucleic acid molecule inhibiting the (over)expression of a TBC1D7 gene in a cell, which over-expresses the gene, which molecule includes a sense strand and an antisense strand complementary thereto, hybridized to each other to form the double-stranded nucleic acid molecule and targets sequence of SEQ ID NOs: 3 and/or 4, for manufacturing a pharmaceutical composition for treating a cancer (over)expressing the TBC1D7 gene, including lung and esophageal cancers.

The present invention also provides the double-stranded nucleic acid molecules of the present invention for use in treating a cancer (over)expressing the TBC1D7 gene.

The present invention further provides a method or process for manufacturing a pharmaceutical composition for treating a cancer (over)expressing the TBC1D7 gene, wherein the method or process includes step for formulating a pharmaceutically or physiologically acceptable carrier with a double-stranded nucleic acid molecule inhibiting the (over)expression of a TBC1D7 gene in a cell, which over-expresses the gene, which molecule includes a sense strand and an antisense strand complementary thereto, hybridized to each other to form the double-stranded nucleic acid molecule and targets sequence of SEQ ID NOs: 3 and/or 4 as active ingredients.

The present invention also provides a method or process for manufacturing a pharmaceutical composition for treating a cancer (over)expressing the TBC1D7 gene, wherein the method or process includes step for admixing an active ingredient with a pharmaceutically or physiologically acceptable carrier, wherein the active ingredient is a double-stranded nucleic acid molecule inhibiting the expression of TBC1D7 gene in a cell, which over-expresses the gene, which molecule includes a sense strand and an antisense strand complementary thereto, hybridized to each other to form the double-stranded nucleic acid molecule and targets target sequence of SEQ ID NOs: 3 and/or 4.

(5) Method for Diagnosing TBC1D7-Mediated Cancers

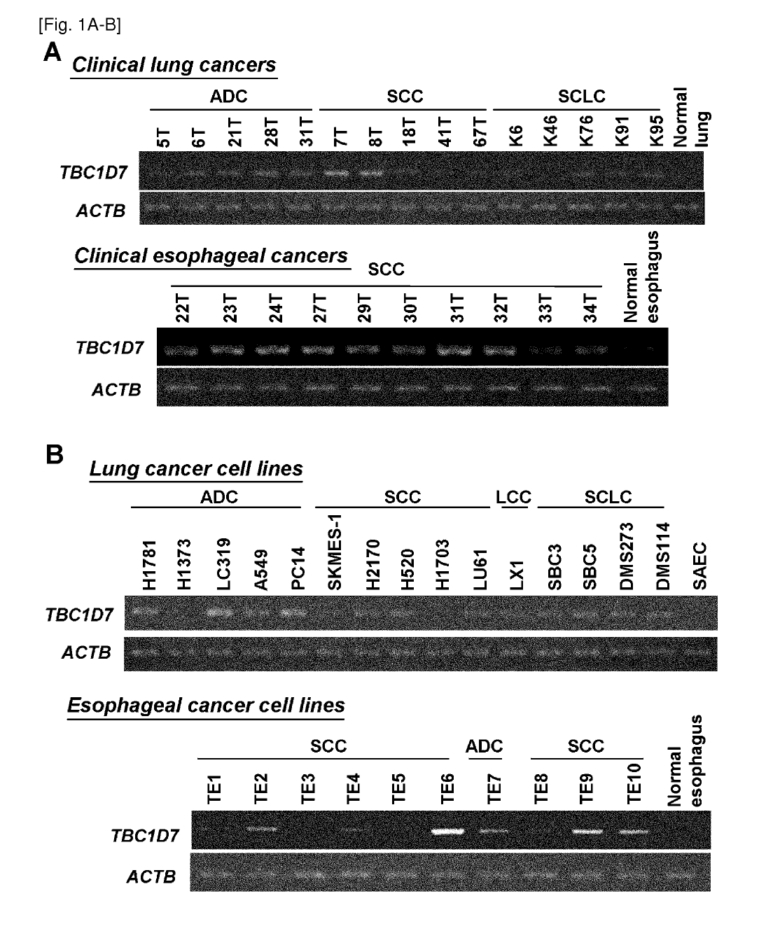

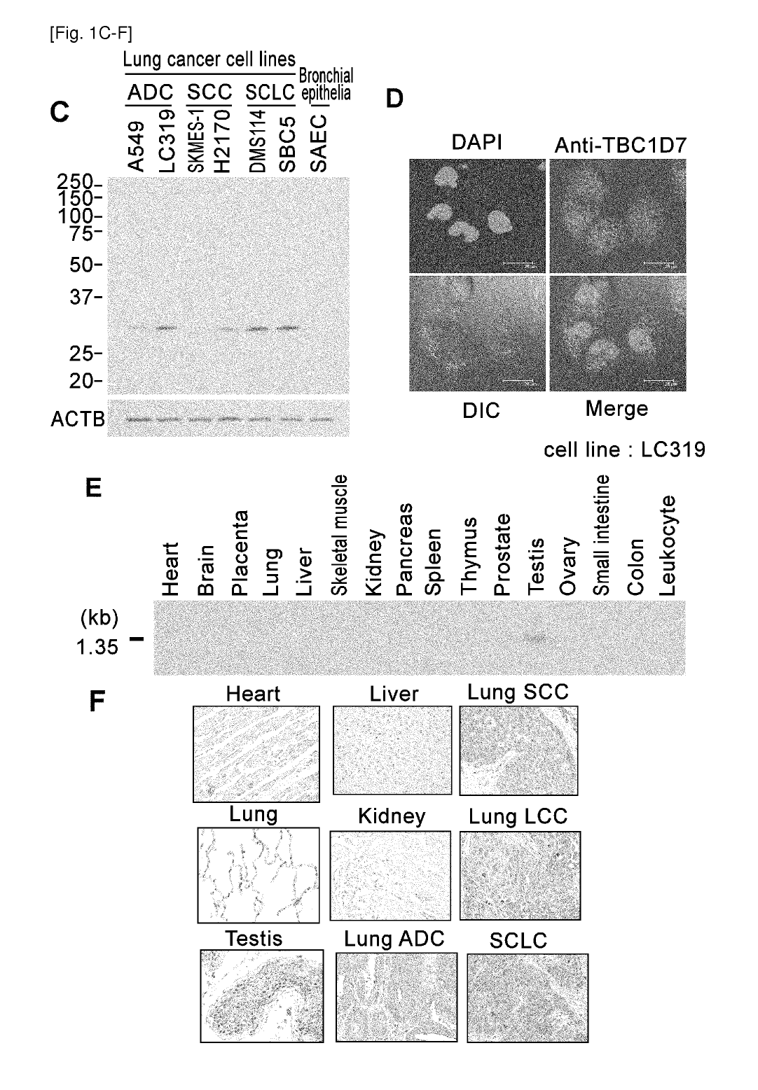

The expression of TBC1D7 gene was found to be specifically elevated in lung and esophageal cancers tissues compared with corresponding normal tissues (FIG. 1). Therefore, the gene identified herein as well as its transcription and translation products have diagnostic utility as markers for cancers mediated by a TBC1D7 gene and by measuring the expression of the TBC1D7 gene in a sample derived from a patient suspected to be suffering from cancers. These cancers can be diagnosed or detected by comparing the expression level of TBC1D7 between the subject-derived sample with a normal sample. Specifically, the present invention provides a method for diagnosing or detecting cancers mediated by TBC1D7 by determining the expression level of TBC1D7 in the subject. The TBC1D7-promoted cancers that can be diagnosed or detected by the present method include lung and esophageal cancers. Lung cancers include non-small lung cancer and small lung cancer.

Alternatively, the present invention provides a method for detecting or identifying cancer cells in a subject-derived tissue sample, said method including the step of determining the expression level of the TBC1D7 gene in a subject-derived tissue sample, wherein an increase in said expression level as compared to a normal control level of said gene indicates the presence or suspicion of cancer cells in the tissue. Preferably, the tissue is a lung or esophageal tissue.

According to the present invention, an intermediate result for examining the condition of a subject can be provided. Such intermediate result can be combined with additional information to assist a doctor, nurse, or other practitioner to diagnose that a subject suffers from the disease. Alternatively, the present invention can be used to detect cancerous cells in a subject-derived tissue, and provide a doctor with useful information to diagnose that the subject suffers from the disease.

For example, according to the present invention, when there is doubt regarding the presence of cancer cells in the tissue obtained from a subject, clinical decisions can be reached by considering the expression level of the TBC1D7 gene, plus a different aspect of the disease including tissue pathology, levels of known tumor marker(s) in blood, and clinical course of the subject, etc. For example, some well-known diagnostic lung and esophageal cancer markers in blood include ACT, CA19-9, CA50, CA72-4, CA130, CA602, CEA, DUPAN-2, IAP, KMO-1, NSE, SCC, SLX, Span-1, STN, TPA, cytokeratin 19 fragment, and CYFRA 21-1. Namely, in this particular embodiment of the present invention, the outcome of the gene expression analysis serves as an intermediate result for further diagnosis of a subject's disease state.

In another embodiment, the present invention provides a method for detecting a diagnostic marker of cancer, said method including the step of detecting the expression of the TBC1D7 gene in a subject-derived biological sample as a diagnostic marker of cancer (for example, lung or esophageal cancer).

Specifically, the present invention provides the following methods [1] to [10]:

[1] A method for diagnosing cancers, e.g., cancers mediated or promoted by a TBC1D7, wherein said method including the steps of:

(a) detecting the expression level of TBC1D7 in a biological sample; and

(b) relating an increase of the expression level compared to a normal control level of the gene to the disease.

[2] The method of [1], wherein the expression level is at least 10% greater than normal control level.

[3] The method of [2], wherein the expression level is detected by any one of the method select from the group consisting of:

(a) detecting the mRNA encoding the TBC1D7 polypeptide;

(b) detecting the TBC1D7 polypeptide; and

(c) detecting the biological activity of the TBC1D7 polypeptide.

The method of [1], wherein the cancer results from overexpression of a TBC1D7, or is mediated or promoted by a TBC1D7.

[4] The method of [1], wherein the cancers is lung cancer or esophageal caner.

[5] The method of [4], wherein the lung cancer is non-small cell lung cancer or small cell lung cancer.

[6] The method of [3], wherein the expression level is determined by detecting a hybridization of probe to the gene transcript encoding the TBC1D7 polypeptide.

[7] The method of [3], wherein the expression level is determined by detecting a binding of an antibody against the TBC1D7 polypeptide.

[8] The method of [1], wherein the biological sample includes biopsy, sputum or blood.

[9] The method of [1], wherein the subject-derived biological sample includes an epithelial cell, serum, pleural effusion or esophageal mucosa.

[10] The method of [1], wherein the subject-derived biological sample includes a cancer cell.

[11] The method of [1], wherein the subject-derived biological sample includes a cancerous epithelial cell.

The method of diagnosing cancers will be described in more detail below.

A subject to be diagnosed by the present method is can be a mammal. Exemplary mammals include, but are not limited to, e.g., human, non-human primate, mouse, rat, dog, cat, horse, and cow.

In performing the present methods, a biological sample is collected from the subject to be diagnosed to perform the diagnosis. Any biological material can be used as the biological sample for the determination so long as it includes the objective transcription or translation product of TBC1D7 gene. The biological samples include, but are not limited to, bodily tissues and fluids, for example, blood, e.g. serum, sputum, urine and pleural effusion. In some embodiments, the biological sample contains a cell population including an epithelial cell, for example, a cancerous epithelial cell or an epithelial cell derived from tissue suspected to be cancerous. Further, if necessary, the cell can be purified from the obtained bodily tissues and fluids, and then used as the biological sample.

According to the present invention, the expression level of TBC1D7 gene in the subject-derived biological sample is determined. The expression level can be determined at the transcription (nucleic acid) product level, using methods known in the art. For example, the mRNA of TBC1D7 gene can be quantified using probes by hybridization methods (e.g. Northern blot analysis). The detection can be carried out on a chip or an array. The use of an array can be for detecting the expression level of a plurality of genes (e.g., various cancer specific genes) including TBC1D7 gene. Those skilled in the art can prepare such probes utilizing the sequence information of the TBC1D7 (SEQ ID NO: 1; GenBank Accession No. NM_{—}016495). For example, the cDNA of TBC1D7 gene can be used as a probe. If necessary, the probe can be labeled with a suitable label, for example, dyes, fluorescent and isotopes, and the expression level of the gene can be detected as the intensity of the hybridized labels (see, (c) Northern-blot analysis in [EXAMPLE1]).

Furthermore, the transcription product of TBC1D7 gene can be quantified using primers by amplification-based detection methods (e.g., RT-PCR). Such primers can also be prepared based on the available sequence information of the gene. For example, the primers (SEQ ID NO: 5 and 6) used in the Example can be employed for the detection by RT-PCR or Northern blot, but the present invention is not restricted thereto (see, (b) Semi-quantitative RT-PCR and (c) Northern -blot analysis in [EXAMPLE1]).

Specifically, a probe or primer used for the present method hybridizes under stringent, moderately stringent, or low stringent conditions to the mRNA of TBC1D7 gene.

Alternatively, the translation product can be detected for the diagnosis of the present invention. For example, the quantity of TBC1D7 protein can be determined. A method for determining the quantity of the protein as the translation product includes immunoassay methods that use an antibody specifically recognizing the protein. The antibody can be monoclonal or polyclonal. Furthermore, any fragment or modification (e.g., chimeric antibody, scFv, Fab, F(ab′)2, Fv, etc.) of the antibody can be used for the detection, so long as the fragment retains the binding ability to TBC1D7 protein. Methods to prepare these kinds of antibodies for the detection of proteins are well known in the art, and any method can be employed in the present invention to prepare such antibodies and equivalents thereof (see, (2) Antibody in Definition).

As another method to detect the expression level of TBC1D7 based on its translation product, the intensity of staining can be observed via immunohistochemical analysis using an antibody against TBC1D7 protein. Namely, the observation of strong staining indicates increased presence of the protein and at the same time high expression level of TBC1D7 (see, (g) Immunohistochemistry and Tissue-microarray analysis in [EXAMPLE 1]).

Moreover, in addition to the expression level of TBC1D7, the expression level of other cancer-associated genes, for example, genes known to be differentially expressed in cancers can also be determined to improve the accuracy of the diagnosis.

The expression level of cancer marker gene including TBC1D7 in a biological sample can be considered to be increased if it increases from the control level of the corresponding cancer marker gene (e.g., in a normal or non-cancerous cell) by, for example, 10%, 25%, or 50%; or increases to more than 1.1 fold, more than 1.5 fold, more than 2.0 fold, more than 5.0 fold, more than 10.0 fold, or more.

The control level can be determined at the same time with the test biological sample by using a sample(s) previously collected and stored from a subject/subjects whose disease state (cancerous or non-cancerous) is/are known. Alternatively, the control level can be determined by a statistical method based on the results obtained by analyzing previously determined expression level(s) of TBC1D7 in samples from subjects whose disease state are known. Furthermore, the control level can be a database of expression patterns from previously tested cells. Moreover, according to an aspect of the present invention, the expression level of a TBC1D7 in a biological sample can be compared to multiple control levels, which control levels are determined from multiple reference samples. In some embodiments, a control level determined from a reference sample derived from a tissue type similar to that of the patient-derived biological sample is used. In some embodiments, the standard value of the expression levels of TBC1D7 in a population with a known disease state is used. The standard value can be obtained by any method known in the art. For example, a range of mean +/−2 S.D. or mean +/−3 S.D. can be used as standard value.

In the context of the present invention, a control level determined from a biological sample that is known not to be cancerous is called “normal control level”. On the other hand, if the control level is determined from a cancerous biological sample, it will be called “cancerous control level”.

When the expression level of TBC1D7 is increased compared to the normal control level or is similar to the cancerous control level, the subject can be diagnosed to be suffering from or at a risk of developing cancer, e.g., a cancer that is mediated by or results from overexpression of TBC1D7. Furthermore, in case where the expression levels of TBC1D7 gene are compared, a similarity in the gene expression pattern between the sample and the reference which is cancerous indicates that the subject is suffering from or at a risk of developing cancer, e.g., a cancer that is mediated by or results from overexpression of a TBC1D7.

Difference between the expression levels of a test biological sample and the control level can be normalized to the expression level of control nucleic acids, e.g., housekeeping genes, whose expression levels are known not to differ depending on the cancerous or non-cancerous state of the cell. Exemplary control genes include, but are not limited to, beta-actin, glyceraldehyde 3 phosphate dehydrogenase, and ribosomal protein P1.

(6) Method for Assessing the Prognosis of TBC1D7 Mediated Cancer

The present invention is based, in part, on the discovery that TBC1D7 (over)expression is significantly associated with poorer prognosis of patients with TBC1D7-mediated cancers, e.g., lung or esophageal cancers. Thus, the present invention provides a method for determining or assessing the prognosis of a patient with cancer, e.g., a cancer mediated by or resulting from overexpression of a TBC1D7, e.g, lung cancer and/or esophageal cancer, by detecting the expression level of the TBC1D7 gene in a biological sample of the patient; comparing the detected expression level to a control level; and determining a increased expression level to the control level as indicative of poor prognosis (poor survival).

Herein, the term “prognosis” refers to a forecast as to the probable outcome of the disease as well as the prospect of recovery from the disease as indicated by the nature and symptoms of the case. Accordingly, a less favorable, negative or poor prognosis is defined by a lower post-treatment survival term or survival rate. Conversely, a positive, favorable, or good prognosis is defined by an elevated post-treatment survival term or survival rate. The terms “assessing the prognosis” refer to the ability of predicting, forecasting or correlating a given detection or measurement with a future outcome of cancer of the patient (e.g., malignancy, likelihood of curing cancer, estimated time of survival, and the like). For example, a determination of the expression level of TBC1D7 over time enables a predicting of an outcome for the patient (e.g., increase or decrease in malignancy, increase or decrease in grade of a cancer, likelihood of curing cancer, survival, and the like). In the context of the present invention, the phrase “assessing (or determining) the prognosis” is intended to encompass predictions and likelihood analysis of cancer, progression, particularly cancer recurrence, metastatic spread and disease relapse. The present method for assessing prognosis is intended to be used clinically in making decisions concerning treatment modalities, including therapeutic intervention, diagnostic criteria for example, disease staging, and disease monitoring and surveillance for metastasis or recurrence of neoplastic disease.

The patient-derived biological sample used for the method can be any sample derived from the subject to be assessed so long as the TBC1D7 gene can be detected in the sample. In some embodiments, the biological sample includes a lung cell (a cell obtained from lung or esophageal). Furthermore, the biological sample includes bodily fluids for example, sputum, blood, serum, plasma, pleural effusion, esophageal mucosa, and so on. Moreover, the sample can be cells purified from a tissue. The biological samples can be obtained from a patient at various time points, including before, during, and/or after a treatment.

According to the present invention, it was shown that the higher the expression level of the TBC1D7 gene measured in the patient-derived biological sample, the poorer the prognosis for post-treatment remission, recovery, and/or survival and the higher the likelihood of poor clinical outcome. Thus, according to the present method, the “control level” used for comparison can be, for example, the expression level of the TBC1D7 gene detected before any kind of treatment in an individual or a population of individuals who showed good or positive prognosis of cancer, after the treatment, which herein will be referred to as “good prognosis control level”. Alternatively, the “control level” can be the expression level of the TBC1D7 gene detected before any kind of treatment in an individual or a population of individuals who showed poor or negative prognosis of cancer, after the treatment, which herein will be referred to as “poor prognosis control level”. The “control level” is a single expression pattern derived from a single reference population or from a plurality of expression patterns. Thus, the control level can be determined based on the expression level of the TBC1D7 gene detected before any kind of treatment in a patient of cancer, or a population of the patients whose disease state (good or poor prognosis) is known. In some embodiments, the cancer is lung cancer. In some embodiments, the standard value of the expression levels of the TBC1D7 gene in a patient group with a known disease state is used. The standard value can be obtained by any method known in the art. For example, a range of mean +/−2 S.D. or mean +/−3 S.D. can be used as standard value.

The control level can be determined at the same time with the test biological sample by using a sample(s) previously collected and stored before any kind of treatment from cancer patient(s) (control or control group) whose disease state (good prognosis or poor prognosis) are known.

Alternatively, the control level can be determined by a statistical method based on the results obtained by analyzing the expression level of the TBC1D7 gene in samples previously collected and stored from a control group. Furthermore, the control level can be a database of expression patterns from previously tested cells or patients. Moreover, according to an aspect of the present invention, the expression level of the TBC1D7 gene in a biological sample can be compared to multiple control levels, which control levels are determined from multiple reference samples. In some embodiments, a control level determined from a reference sample derived from a tissue type similar to that of the patient-derived biological sample is used.

According to the present invention, a similarity in the expression level of the

TBC1D7 gene to the good prognosis control level indicates a more favorable prognosis of the patient and an increase in the expression level in comparison to the good prognosis control level indicates less favorable, poorer prognosis for post-treatment remission, recovery, survival, and/or clinical outcome. On the other hand, a decrease in the expression level of the TBC1D7 gene in comparison to the poor prognosis control level indicates a more favorable prognosis of the patient and a similarity in the expression level to the poor prognosis control level indicates less favorable, poorer prognosis for post-treatment remission, recovery, survival, and/or clinical outcome.

An expression level of the TBC1D7 gene in a biological sample can be considered altered (i.e., increased or decreased) when the expression level differs from the control level by more than 1.0, 1.5, 2.0, 5.0, 10.0, or more fold.

The difference in the expression level between the test biological sample and the control level can be normalized to a control, e.g., housekeeping gene. For example, polynucleotides whose expression levels are known not to differ between the cancerous and non-cancerous cells, including those coding for beta-actin, glyceraldehyde 3-phosphate dehydrogenase, and ribosomal protein P1, can be used to normalize the expression levels of the TBC1D7 gene.

The expression level can be determined by detecting the gene transcript in the patient-derived biological sample using techniques well known in the art. The gene transcripts detected by the present method include both the transcription and translation products, for example, mRNA and protein. For instance, the transcription product of the TBC1D7 gene can be detected by hybridization, e.g., Northern blot hybridization analyses, that use a TBC1D7 gene probe to the gene transcript. The detection can be carried out on a chip or an array. An array can be used for detecting the expression level of a plurality of genes including the TBC1D7 gene. As another example, amplification-based detection methods, for example, reverse-transcription based polymerase chain reaction (RT-PCR) which use primers specific to the TBC1D7 gene can be employed for the detection (see (b) Semi-quantitative RT-PCR in [EXAMPLE 1]). The TBC gene-specific probe or primers can be designed and prepared using conventional techniques by referring to the whole sequence of the TBC1D7 (SEQ ID NO: 1). For example, the primers (SEQ ID NOs: 5 and 6) used in the Example can be employed for the detection by RT-PCR, but the present invention is not restricted thereto.

Specifically, a probe or primer used for the present method hybridizes under stringent, moderately stringent, or low stringent conditions to the mRNA of the TBC1D7 gene. As used herein, the phrase “stringent (hybridization) conditions” refers to conditions under which a probe or primer will hybridize to its target sequence, but to no other sequences. Stringent conditions are sequence-dependent and will be different under different circumstances. Specific hybridization of longer sequences is observed at higher temperatures than shorter sequences. Generally, the temperature of a stringent condition is selected to be about 5degree Centigrade lower than the thermal melting point (Tm) for a specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30degree Centigrade for short probes or primers (e.g., 10 to 50 nucleotides) and at least about 60degree Centigrade for longer probes or primers. Stringent conditions can also be achieved with the addition of destabilizing agents, for example, formamide.

Alternatively, the translation product can be detected for the assessment of the present invention. For example, the quantity of the TBC1D7 protein can be determined. A method for determining the quantity of the protein as the translation product includes immunoassay methods that use an antibody specifically recognizing the TBC1D7 protein. The antibody can be monoclonal or polyclonal. Furthermore, any fragment or modification (e.g., chimeric antibody, scFv, Fab, F(ab′)2, Fv, etc.) of the antibody can be used for the detection, so long as the fragment retains the binding ability to the TBC1D7 protein. Methods to prepare these kinds of antibodies for the detection of proteins are well known in the art, and any method can be employed in the present invention to prepare such antibodies and equivalents thereof. As another method to detect the expression level of the TBC1D7 gene based on its translation product, the intensity of staining can be observed via immunohistochemical analysis using an antibody against TBC1D7 protein. Namely, the observation of strong staining indicates increased presence of the TBC1D7 protein and at the same time high expression level of the TBC1D7 gene.

Furthermore, the TBC1D7 protein is known to have a cell proliferating activity.

Therefore, the expression level of the TBC1D7 gene can be determined using such cell proliferating activity as an index. For example, cells which express TBC1D7 are prepared and cultured in the presence of a biological sample, and then by detecting the speed of proliferation, or by measuring the cell cycle or the colony forming ability the cell proliferating activity of the biological sample can be determined.

Moreover, in addition to the expression level of the TBC1D7 gene, the expression level of other lung cell-associated genes, for example, genes known to be differentially expressed in lung cancer or esophageal cancer, can also be determined to improve the accuracy of the assessment. Such other lung cancer-associated genes include those described in WO 2004/031413 and WO 2005/090603; and such other esophageal cancer-associated genes include those described in WO 2007/013671.

The patient to be assessed for the prognosis of cancer according to the method can be a mammal and includes human, non-human primate, mouse, rat, dog, cat, horse, and cow.

Alternatively, according to the present invention, an intermediate result can also be provided in addition to other test results for assessing the prognosis of a subject. Such intermediate result can assist a doctor, nurse, or other practitioner to assess, determine, or estimate the prognosis of a subject. Additional information that can be considered, in combination with the intermediate result obtained by the present invention, to assess prognosis includes clinical symptoms and physical conditions of a subject.

(7) Kits for Diagnosing Cancer or Assessing the Prognosis of Cancer

The present invention provides a kit for diagnosing cancer or assessing the prognosis of cancer. In some embodiments, the cancer is mediated by TBC1D7 or resulting from overexpression of TBC1D7, e.g., lung cancer and/or esophageal cancer. Specifically, the kit includes at least one reagent for detecting the expression of the TBC1D7 gene in a patient-derived biological sample, which reagent can be selected from the group of:

(a) a reagent for detecting mRNA of the TBC1D7 gene;

(b) a reagent for detecting the TBC1D7 protein; and

(c) a reagent for detecting the biological activity of the TBC1D7 protein.

Suitable reagents for detecting mRNA of the TBC1D7 gene include nucleic acids that specifically bind to or identify the TBC1D7 mRNA, for example, oligonucleotides which have a complementary sequence to a part of the TBC1D7 mRNA. These kinds of oligonucleotides are exemplified by primers and probes that are specific to the TBC1D7 mRNA. These kinds of oligonucleotides can be prepared based on methods well known in the art. If needed, the reagent for detecting the TBC1D7 mRNA can be immobilized on a solid matrix. Moreover, more than one reagent for detecting the TBC1D7 mRNA can be included in the kit.

On the other hand, suitable reagents for detecting the TBC1D7 protein include antibodies to the TBC1D7 protein. The antibody can be monoclonal or polyclonal. Furthermore, any fragment or modification (e.g., chimeric antibody, scFv, Fab, F(ab′)2, Fv, etc.) of the antibody can be used as the reagent, so long as the fragment retains the binding ability to the TBC1D7 protein. Methods to prepare these kinds of antibodies for the detection of proteins are well known in the art, and any method can be employed in the present invention to prepare such antibodies and equivalents thereof. Furthermore, the antibody can be labeled with signal generating molecules via direct linkage or an indirect labeling technique. Labels and methods for labeling antibodies and detecting the binding of antibodies to their targets are well known in the art and any labels and methods can be employed for the present invention. Moreover, more than one reagent for detecting the TBC1D7 protein can be included in the kit.

Furthermore, the biological activity can be determined by, for example, measuring the cell proliferating activity due to the expressed TBC1D7 protein in the biological sample. For example, the cell is cultured in the presence of a patient-derived biological sample, and then by detecting the speed of proliferation, or by measuring the cell cycle or the colony forming ability the cell proliferating activity of the biological sample can be determined. If needed, the reagent for detecting the TBC1D7 mRNA can be immobilized on a solid matrix. Moreover, more than one reagent for detecting the biological activity of the TBC1D7 protein can be included in the kit.

The kit can include more than one of the aforementioned reagents. Furthermore, the kit can include a solid matrix and reagent for binding a probe against the TBC1D7 gene or antibody against the TBC1D7 protein, a medium and container for culturing cells, positive and negative control reagents, and a secondary antibody for detecting an antibody against the TBC1D7 protein. For example, tissue samples obtained from patient with good prognosis or poor prognosis can serve as useful control reagents. A kit of the present invention can further include other materials desirable from a commercial and user standpoint, including buffers, diluents, filters, needles, syringes, and package inserts (e.g., written, tape, CD-ROM, etc.) with instructions for use. These reagents and such can be included in a container with a label. Suitable containers include bottles, vials, and test tubes. The containers can be formed from a variety of materials, for example, glass or plastic.

As an embodiment of the present invention, when the reagent is a probe against the TBC1D7 mRNA, the reagent can be immobilized on a solid matrix, for example, a porous strip, to form at least one detection site. The measurement or detection region of the porous strip can include a plurality of sites, each containing a nucleic acid (probe). A test strip can also contain sites for negative and/or positive controls. Alternatively, control sites can be located on a strip separated from the test strip. Optionally, the different detection sites can contain different amounts of immobilized nucleic acids, i.e., a higher amount in the first detection site and lesser amounts in subsequent sites. Upon the addition of test sample, the number of sites displaying a detectable signal provides a quantitative indication of the amount of TBC1D7 mRNA present in the sample. The detection sites can be configured in any suitably detectable shape and are typically in the shape of a bar or dot spanning the width of a test strip.

The kit of the present invention can further include a positive control sample or TBC1D7 standard sample. The positive control sample of the present invention can be prepared by collecting TBC1D7 positive blood samples and then those TBC1D7 level are assayed. Alternatively, purified TBC1D7 protein or polynucleotide can be added to TBC1D7 free serum to form the positive sample or the TBC1D7 standard. In the present invention, purified TBC1D7 can be recombinant protein. The TBC1D7 level of the positive control sample is, for example more than cut off value.

Hereinafter, the present invention is described in more detail with reference to the Examples. However, the following materials, methods and examples only illustrate aspects of the invention and in no way are intended to limit the scope of the present invention. As such, methods and materials similar or equivalent to those described herein can be used in the practice or testing of the present invention.

Screening Methods

(1) Test Compounds for Screening

In the context of the present invention, agents to be identified through the present screening methods can be any compound or composition. Furthermore, the test agent or compound exposed to a cell or protein according to the screening methods of the present invention can be a single compound or a combination of compounds. When a combination of compounds is used in the methods, the compounds can be contacted sequentially or simultaneously.

Any test agent or compound, for example, cell extracts, cell culture supernatant, products of fermenting microorganism, extracts from marine organism, plant extracts, purified or crude proteins, peptides, non-peptide compounds, synthetic micro-molecular compounds (including nucleic acid constructs, for example, antisense DNA, siRNA, ribozymes, etc.) and natural compounds can be used in the screening methods of the present invention. The test agent or compound of the present invention can be also obtained using any of the numerous approaches in combinatorial library methods known in the art, including
(1) biological libraries,
(2) spatially addressable parallel solid phase or solution phase libraries,
(3) synthetic library methods requiring deconvolution,
(4) the “one-bead one-compound” library method and
(5) synthetic library methods using affinity chromatography selection.

The biological library methods using affinity chromatography selection is limited to peptide libraries, while the other four approaches are applicable to peptide, non-peptide oligomer or small molecule libraries of compounds (Lam, Anticancer Drug Des 1997, 12: 145-67). Examples of methods for the synthesis of molecular libraries can be found in the art (DeWitt et al., Proc Natl Acad Sci USA 1993, 90: 6909-13; Erb et al., Proc Natl Acad Sci USA 1994, 91: 11422-6; Zuckermann et al., J Med Chem 37: 2678-85, 1994; Cho et al., Science 1993, 261: 1303-5; Carell et al., Angew Chem Int Ed Engl 1994, 33: 2059; Carell et al., Angew Chem Int Ed Engl 1994, 33: 2061; Gallop et al., J Med Chem 1994, 37: 1233-51). Libraries of compounds can be presented in solution (see Houghten, Bio/Techniques 1992, 13: 412-21) or on beads (Lam, Nature 1991, 354: 82-4), chips (Fodor, Nature 1993, 364: 555-6), bacteria (U.S. Pat. No. 5,223,409), spores (U.S. Pat. Nos. 5,571,698; 5,403,484 and 5,223,409), plasmids (Cull et al., Proc Natl Acad Sci USA 1992, 89: 1865-9) or phage (Scott and Smith, Science 1990, 249: 386-90; Devlin, Science 1990, 249: 404-6; Cwirla et al., Proc Natl Acad Sci USA 1990, 87: 6378-82; Felici, J Mol Biol 1991, 222: 301-10; US Pat. Application 2002-103360).

A compound in which a part of the structure of the compound screened by any of the present screening methods is converted by addition, deletion and/or replacement, is included in the agents obtained by the screening methods of the present invention.

Furthermore, when the screened test agent or compound is a protein, for obtaining a DNA encoding the protein, either the whole amino acid sequence of the protein can be determined to deduce the nucleic acid sequence coding for the protein, or partial amino acid sequence of the obtained protein can be analyzed to prepare an oligo DNA as a probe based on the sequence, and screen cDNA libraries with the probe to obtain a DNA encoding the protein. The obtained DNA finds use in preparing the test agent or compound which is a candidate for treating or preventing cancer.

Test agents or compounds useful in the screening described herein can also be antibodies or non-antibody binding proteins that specifically bind to the TBC1D7 protein or partial TBC1D7 peptides that lack the activity to binding for partner. Such partial protein or antibody can be prepared by the methods described herein (see (1) Cancer-related genes and cancer-related protein, and functional equivalent thereof in Definition or Antibodies) and can be tested for their ability to block binding of the protein with its binding partners.

(i) Molecular Modeling

Construction of test agent/compound libraries is facilitated by knowledge of the molecular structure of compounds known to have the properties sought, and/or the molecular structure of the target molecules to be inhibited. One approach to preliminary screening of test agents or compounds suitable for further evaluation is computer modeling of the interaction between the test agent/compound and its target.

Computer modeling technology allows the visualization of the three-dimensional atomic structure of a selected molecule and the rational design of new compounds that will interact with the molecule. The three-dimensional construct typically depends on data from x-ray crystallographic analysis or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

An example of the molecular modeling system described generally above includes the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, for example, Rotivinen et al. Acta Pharmaceutica Fennica 1988, 97: 159-66; Ripka, New Scientist 1988, 54-8; McKinlay & Rossmann, Annu Rev Pharmacol Toxiciol 1989, 29: 111-22; Perry & Davies, Prog Clin Biol Res 1989, 291: 189-93; Lewis & Dean, Proc R Soc Lond 1989, 236: 125-40, 141-62; and, with respect to a model receptor for nucleic acid components, Askew et al., J Am Chem Soc 1989, 111: 1082-90.

Other computer programs that screen and graphically depict chemicals are available from companies for example, BioDesign, Inc., Pasadena, Calif., Allelix, Inc, Mississauga, Ontario, Canada, and Hypercube, Inc., Cambridge, Ontario. See, e.g., DesJarlais et al., J Med Chem 1988, 31: 722-9; Meng et al., J Computer Chem 1992, 13: 505-24; Meng et al., Proteins 1993, 17: 266-78; Shoichet et al., Science 1993, 259: 1445-50.

Once an inhibitor of the TBC1D7 activity has been identified, combinatorial chemistry techniques can be employed to construct any number of variants based on the chemical structure of the identified inhibitor, as detailed below. The resulting library of candidate inhibitors, or “test agents or compounds” can be screened using the methods of the present invention to identify test agents or compounds of the library that disrupt the TBC1D7 activity.

(ii) Combinatorial Chemical Synthesis

Combinatorial libraries of test agents or compounds can be produced as part of a rational drug design program involving knowledge of core structures existing in known inhibitors of the TBC1D7 activity. This approach allows the library to be maintained at a reasonable size, facilitating high throughput screening. Alternatively, simple, particularly short, polymeric molecular libraries can be constructed by simply synthesizing all permutations of the molecular family making up the library. An example of this latter approach would be a library of all peptides six amino acids in length. Such a peptide library could include every 6 amino acid sequence permutation. This type of library is termed a linear combinatorial chemical library.

Preparation of combinatorial chemical libraries is well known to those of skill in the art, and can be generated by either chemical or biological synthesis. Combinatorial chemical libraries include, but are not limited to, peptide libraries (see, e.g., U.S. Pat. No. 5,010,175; Furka, Int J Pept Prot Res 1991, 37: 487-93; Houghten et al., Nature 1991, 354: 84-6). Other chemistries for generating chemical diversity libraries can also be used. Such chemistries include, but are not limited to: peptides (e.g., PCT Publication No. WO 91/19735), encoded peptides (e.g., WO 93/20242), random bio-oligomers (e.g., WO 92/00091), benzodiazepines (e.g., U.S. Pat. No. 5,288,514), diversomers for example, hydantoins, benzodiazepines and dipeptides (DeWitt et al., Proc Natl Acad Sci USA 1993, 90:6909-13), vinylogous polypeptides (Hagihara et al., J Amer Chem Soc 1992, 114: 6568), nonpeptidal peptidomimetics with glucose scaffolding (Hirschmann et al., J Amer Chem Soc 1992, 114: 9217-8), analogous organic syntheses of small compound libraries (Chen et al., J. Amer Chem Soc 1994, 116: 2661), oligocarbamates (Cho et al., Science 1993, 261: 1303), and/or peptidylphosphonates (Campbell et al., J Org Chem 1994, 59: 658), nucleic acid libraries (see Ausubel, Current Protocols in Molecular Biology, 1990-2008, John Wiley Interscience; Sambrook and Russell, Molecular Cloning: A Laboratory Manual, 3^{rd} Ed., 2001, Cold Spring Harbor Laboratory, New York, USA), peptide nucleic acid libraries (see, e.g., U.S. Pat. No. 5,539,083), antibody libraries (see, e.g., Vaughan et al., Nature Biotechnology 1996, 14(3):309-14 and PCT/US96/10287), carbohydrate libraries (see, e.g., Liang et al., Science 1996, 274: 1520-22; US Patent 5,593,853), and small organic molecule libraries (see, e.g., benzodiazepines, Gordon E M. Curr Opin Biotechnol. 1995 Dec. 1; 6(6):624-31.; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

(iii) Other Candidates

Another approach uses recombinant bacteriophage to produce libraries. Using the “phage method” (Scott & Smith, Science 1990, 249: 386-90; Cwirla et al., Proc Natl Acad Sci USA 1990, 87: 6378-82; Devlin et al., Science 1990, 249: 404-6), very large libraries can be constructed (e.g., 106 -108 chemical entities). A second approach uses primarily chemical methods, of which the Geysen method (Geysen et al., Molecular Immunology 1986, 23: 709-15; Geysen et al., J Immunologic Method 1987, 102: 259-74); and the method of Fodor et al. (Science 1991, 251: 767-73) are examples. Furka et al. (14th International Congress of Biochemistry 1988, Volume #5, Abstract FR:013; Furka, Int J Peptide Protein Res 1991, 37: 487-93), Houghten (U.S. Pat. No. 4,631,211) and Rutter et al. (U.S. Pat. No. 5,010,175) describe methods to produce a mixture of peptides that can be tested as agonists or antagonists.

Aptamers are macromolecules composed of nucleic acid that bind tightly to a specific molecular target. Tuerk and Gold (Science. 249:505-510 (1990)) discloses SELEX (Systematic Evolution of Ligands by Exponential Enrichment) method for selection of aptamers. In the SELEX method, a large library of nucleic acid molecules {e.g., 10¹⁵ different molecules) can be used for screening.

2) Screening Methods

(i) General Screening Method

Compounds that bind to TBC1D7 protein can be screened, for example, by immunoprecipitation. In immunoprecipitation, an immune complex is formed by adding antibodies or non-antibody binding proteins to a cell lysate prepared using an appropriate detergent. The immune complex consists of a polypeptide, a polypeptide having a binding affinity for the polypeptide, and an antibody or non-antibody binding protein. Immunoprecipitation can be also conducted using antibodies against a polypeptide, in addition to using antibodies against the above epitopes, which antibodies can be prepared as described above (see Antibodies).

An immune complex can be precipitated, for example, by Protein A sepharose or Protein G sepharose when the antibody is a mouse IgG antibody. If the polypeptide of the present invention is prepared as a fusion protein with an epitope, for example GST, an immune complex can be formed in the same manner as in the use of the antibody against the polypeptide, using a substance specifically binding to these epitopes, for example glutathione-Sepharose 4B. Immunoprecipitation can be performed by following or according to, for example, the methods in the literature (Harlow and Lane, Antibodies, 511-52, Cold Spring Harbor Laboratory publications, New York (1988)).

SDS-PAGE is commonly used for analysis of immunoprecipitated proteins and the bound protein can be analyzed by the molecular weight of the protein using gels with an appropriate concentration. Since the protein bound to the polypeptide is difficult to detect by a common staining method, for example Coomassie staining or silver staining, the detection sensitivity for the protein can be improved by culturing cells in culture medium containing radioactive isotope, “S-methionine or “S-cysteine, labeling proteins in the cells, and detecting the proteins. The target protein can be purified directly from the SDS-polyacrylamide gel and its sequence can be determined, when the molecular weight of a protein has been revealed.

As a method for screening for proteins that bind to the TBC1D7 polypeptide using the polypeptide, for example, West-Western blotting analysis (Skolnik et al., Cell 65: 83-90 (1991)) can be used. Specifically, a protein binding to the TBC1D7 polypeptide can be obtained by preparing a cDNA library from cells, tissues, organs (see (1) Cancer-related genes and cancer-related protein, and functional equivalent thereof in Definition), or cultured cells expected to express a protein binding to the TBC1D7 polypeptide using a phage vector (e.g., ZAP), expressing the protein on LB-agarose, fixing the protein expressed on a filter, reacting the purified and labeled TBC1D7 polypeptide with the above filter, and detecting the plaques expressing proteins bound to the TBC1D7 polypeptide according to the label. The TBC1D7 polypeptide can be labeled by utilizing the binding between biotin and avidin, or by utilizing an antibody that specifically binds to the TBC1D7 polypeptide, or a peptide or polypeptide (for example, GST) that is fused to the TBC1D7 polypeptide. Methods using radioisotope or fluorescence and such can be also used.

The terms “label” and “detectable label” are used herein to refer to any composition detectable by spectroscopic, photochemical, biochemical, immunochemical, electrical, optical or chemical means. Such labels include biotin for staining with labeled streptavidin conjugate, magnetic beads (e.g., DYNABEADS™), fluorescent dyes (e.g., fluorescein, Texas red, rhodamine, green fluorescent protein, fluorescein isothiocyanate (FITC), and the like), radiolabels (e.g., ³H, ¹²⁵I, ¹³¹I, ³⁵S, ¹⁴C_{,} ³²P, or ³³P), enzymes (e.g., horse radish peroxidase, alkaline phosphatase, beta-galactosidase, beta-glucosidase, and others commonly used in an ELISA), and calorimetric labels for example colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,275,149; and 4,366,241. Means of detecting such labels are well known to those of skill in the art. Thus, for example, radiolabels can be detected using photographic film or scintillation counters, fluorescent markers can be detected using a photodetector to detect emitted light. Enzymatic labels are typically detected by providing the enzyme with a substrate and detecting, the reaction product produced by the action of the enzyme on the substrate, and calorimetric labels are detected by simply visualizing the colored label.

Alternatively, in another embodiment of the screening method of the present invention, a two-hybrid system utilizing cells can be used (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton and Treisman, Cell 68: 597-612 (1992)”, “Fields and Sternglanz, Trends Genet 10: 286-92 (1994)”).

In the two-hybrid system, the polypeptide of the invention is fused to the SRF-binding region or GAL4-binding region and expressed in yeast cells. A cDNA library is prepared from cells expected to express a protein binding to the polypeptide of the invention, such that the library, when expressed, is fused to the VP16 or GAL4 transcriptional activation region. The cDNA library is then introduced into the above yeast cells and the cDNA derived from the library is isolated from the positive clones detected (when a protein binding to the polypeptide of the invention is expressed in yeast cells, the binding of the two activates a reporter gene, making positive clones detectable). A protein encoded by the cDNA can be prepared by introducing the cDNA isolated above to E. coli and expressing the protein.

As a reporter gene, for example, Ade2 gene, lacZ gene, CAT gene, luciferase gene and such can be used in addition to the HIS3 gene.

A compound binding to TBC1D7 polypeptide can also be screened using affinity chromatography. For example, the TBC1D7 polypeptide can be immobilized on a carrier of an affinity column, and a test compound, containing a protein capable of binding to the TBC1D7 polypeptide, is applied to the column. A test compound herein can be, for example, cell extracts, cell lysates, etc. After loading the test compound, the column is washed, and compounds bound to the TBC1D7 polypeptide can be prepared.

When the test compound is a protein, the amino acid sequence of the obtained protein is analyzed, an oligo DNA is synthesized based on the sequence, and cDNA libraries are screened using the oligo DNA as a probe to obtain a DNA encoding the protein.

A biosensor using the surface plasmon resonance phenomenon can be used as a means for detecting or quantifying the bound compound in the present invention. When such a biosensor is used, the interaction between the TBC1D7 polypeptide and a test compound can be observed real-time as a surface plasmon resonance signal, using only a minute amount of polypeptide and without labeling (for example, BIAcore, Pharmacia). Therefore, it is possible to evaluate the binding between the TBC1D7 polypeptide and a test compound using a biosensor, for example, BIAcore.

As a method of screening for compounds that inhibit the binding between a TBC1D7 protein and a binding partner thereof (e.g., RAB17, 14-3-3 zeta, TSC1), many methods well known by one skilled in the art can be used. For example, screening can be carried out as an in vitro assay system, for example, a cellular system. More specifically, first, either the TBC1D7 protein or the binding partner thereof is bound to a support, and the other protein is added together with a test compound thereto. For instance, the RAB17 polypeptide, 14-3-3 zeta polypeptide or TSC1 polypeptide is bound to a support, and the binding partner polypeptide is added together with a test compound thereto. Next, the mixture is incubated, washed and the other protein bound to the support is detected and/or measured. Promising candidate compound can inhibit the binding between the TBC1D7 polypeptide and the above-mentioned binding partner. Here, TBC1D7, RAB17, 14-3-3 zeta, and TSC1 can be prepared not only as a natural protein but also as a recombinant protein prepared by the gene recombination technique. The natural protein can be prepared, for example, by affinity chromatography. On the other hand, the recombinant protein may be prepared by culturing cells transformed with DNA encoding TBC1D7, RAB17, 14-3-3 zeta, or TSC1 to express the protein therein and then recovering it.

The binding between the TBC1D7 polypeptide and the above-mentioned binding partner can be detected or measured using antibodies to TBC1D7 or the binding partner. For example, after contacting a binding partner immobilized on a support with a test compound, and TBC1D7 is added, incubated and washed, and detection or measurement can be conducted using an antibody against TBC1D7 polypeptide. Alternatively, TBC1D7 polypeptide may be immobilized on a support, and an antibody against a binding partner may be used for detection or measurement. In case of using an antibody in the present screening, the antibody is preferably labeled with one of the labeling substances mentioned in this specification, and detected or measured based on the labeling substance. Alternatively, the antibody against TBC1D7 or a binding partner may be used as a primary antibody to be detected with a secondary antibody that is labeled with a labeling substance. Furthermore, the antibody bound to the protein in the screening of the present invention may be detected or measured using protein G or protein A column.

In the context of the present invention, “inhibition of binding” between two proteins refers to at least reducing binding between the proteins. Thus, in some cases, the percentage of binding pairs in a sample in the presence of a test agent or compound will be decreased compared to an appropriate (e.g., not treated with test compound or from a non-cancer sample, or from a cancer sample) control. The reduction in the amount of proteins bound can be, e.g., less than 90%, 80%, 70%, 60%, 50%, 40%, 25%, 10%, 5%, 1% or less (e.g., 0%), than the pairs bound in a control sample.

Examples of supports that can be used for binding proteins include, for example, insoluble polysaccharides, for example, agarose, cellulose and dextran; and synthetic resins, for example, polyacrylamide, polystyrene and silicon; for example, commercial available beads and plates (e.g., multi-well plates, biosensor chip, etc.) prepared from the above materials can be used. When using beads, they can be filled into a column. Alternatively, the use of magnetic beads is also known in the art, and enables one to readily isolate proteins bound on the beads via magnetism.

The binding of a protein to a support can be conducted according to routine methods, for example, chemical bonding and physical adsorption, for example. Alternatively, a protein can be bound to a support via antibodies that specifically recognize the protein. Moreover, binding of a protein to a support can be also conducted by means of avidin and biotin. The binding between proteins is carried out in buffer, for example, but are not limited to, phosphate buffer and Tris buffer, as long as the buffer does not inhibit binding between the proteins.

The methods of screening for molecules that bind when the immobilized polypeptide is exposed to synthetic chemical compounds, or natural substance banks, or a random phage peptide display library, and the methods of screening using high-throughput based on combinatorial chemistry techniques (Wrighton et al., Science 273: 458-63 (1996); Verdine, Nature 384: 11-3 (1996)) to isolate not only proteins but chemical compounds that bind to the protein (including agonist and antagonist) are well known to one skilled in the art.

Furthermore, the phosphorylation level of a polypeptide or functional equivalent thereof can be detected according to any method known in the art. For example, a test compound is contacted with the polypeptide expressing cell, the cell is incubated for a sufficient time to allow phosphorylation of the polypeptide, and then, the amount of phosphorylated polypeptide can be detected. Alternatively, a test compound is contacted with the polypeptide in vitro, the polypeptide is incubated under condition that allows phosphorylation of the polypeptide, and then, the amount of phosphorylated polypeptide can be detected (see (14) In vitro and in vivo kinase assay.).

In the present invention, the conditions suitable for the phosphorylation can be provided with an incubation of substrate and enzyme protein in the presence of phosphate donor, e.g. ATP. The conditions suitable for the phosphorylation also include conditions in culturing cells expressing the polypeptides. For example, the cell is a transformant cell harboring an expression vector including a polynucleotide encoding the TBC1D7 polypeptide (see (1) Cancer-related genes and cancer-related protein, and functional equivalent thereof in Definition). After the incubation, the phosphorylation level of the substrate can be detected, for example, with an antibody recognizing phosphorylated substrate or by detecting labeled gamma-phosphate transferred by the ATP phosphate donor. Prior to the detection of phosphorylated substrate, substrate can be separated from other elements, or cell lysate of transformant cells. For instance, gel electrophoresis can be used for separation of substrate. Alternatively, substrate can be captured by contacting with a carrier having an antibody against substrate.

For detection of phosphorylated protein, SDS-PAGE or immunoprecipitation can be used. Furthermore, an antibody that recognizes a phosphorylated residue or transferred labeled phosphate can be used for detecting phosphorylated protein level. Any immunological techniques using an antibody recognizing the phosphorylated polypeptide can be used for the detection. ELISA or immunoblotting with antibodies recognizing phosphorylated polypeptide can be used for the present invention. When a labeled phosphate donor is used, the phosphorylation level of the substrate can be detected via tracing the label. For example, radio-labeled ATP (e.g. ³²P-ATP) can be used as phosphate donor, wherein radioactivity of the separated substrate correlates with the phosphorylation level of the substrate. Alternatively, an antibody specifically recognizing a phosphorylated substrate from un-phosphorylated substrate can be used for detection phosphorylated substrate.

If the detected amount of phosphorylated TBC1D7 polypeptide contacted with a test compound is decreased to the amount detected in not contacted with the test compound, the test compound is deemed to inhibit polypeptide phosphorylation of TBC1D7 protein and thus have lung cancer and/or esophageal cancer suppressing ability. Herein, a phosphorylation level can be deemed to be “decreased” when it decreases by, for example, 10%, 25%, or 50% from, or at least 0.1 fold, at least 0.2 fold, at least 1 fold, at least 2 fold, at least 5 fold, or at least 10 fold or more compared to that detected for cells not contacted with the test agent or compound. For example, Student's t-test, the Mann-Whitney U-test, or ANOVA can be used for statistical analysis.

Furthermore, the expression level of a polypeptide or functional equivalent thereof can be detected according to any method known in the art. For example, a reporter assay can be used. Suitable reporter genes and host cells are well known in the art. The reporter construct required for the screening can be prepared by using the transcriptional regulatory region of TBC1D7 gene or downstream gene thereof. When the transcriptional regulatory region of the gene has been known to those skilled in the art, a reporter construct can be prepared by using the previous sequence information. When the transcriptional regulatory region remains unidentified, a nucleotide segment containing the transcriptional regulatory region can be isolated from a genome library based on the nucleotide sequence information of the gene. Specifically, the reporter construct required for the screening can be prepared by connecting reporter gene sequence to the transcriptional regulatory region of a TBC1D7 gene of interest. The transcriptional regulatory region of a TBC1D7 gene is the region from a start codon to at least 500bp upstream, for example, 1000 bp, for example, 5000 or 10000 bp upstream. A nucleotide segment containing the transcriptional regulatory region can be isolated from a genome library or can be propagated by PCR. Methods for identifying a transcriptional regulatory region, and also assay protocol are well known (Sambrook and Russell, Molecular Cloning: A Laboratory Manual, 3rd Ed., Chapter 17, 2001, Cold Springs Harbor Laboratory Press).

Various low-throughput and high-throughput enzyme assay formats are known in the art and can be readily adapted for detection or measuring of the phosphorylation level of the TBC1D7 polypeptide. For high-throughput assays, the substrate can conveniently be immobilized on a solid support. Following the reaction, the phosphorylated substrate can be detected on the solid support by the methods described above. Alternatively, the contact step can be performed in solution, after which the substrate can be immobilized on a solid support, and the phosphorylated substrate detected. To facilitate such assays, the solid support can be coated with streptavidin and the substrate labeled with biotin, or the solid support can be coated with antibodies against the substrate. The skilled person can determine suitable assay formats depending on the desired throughput capacity of the screen.

The assays of the invention are also suitable for automated procedures which facilitate high-throughput screening. A number of well-known robotic systems have been developed for solution phase chemistries. These systems include automated workstations like the automated synthesis apparatus developed by Takeda Chemical Industries, Ltd. (Osaka, Japan) and many robotic systems utilizing robotic arms (Zymate II, Zymark Corporation, Hopkinton, Mass.; Orca, Hewlett Packard, Palo Alto, Calif.), which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

(ii) Screening for Compounds that Bind to TBC1D7 Protein(s)

In present invention, over-expression of TBC1D7 in lung cancer and esophageal cancer was detected in spite of no expression in normal organ except testis (FIG. 1). Therefore, using the TBC1D7 gene, proteins encoded by the gene or transcriptional regulatory region of the gene, compounds can be screened that alter the expression of the gene or the biological activity of a polypeptide encoded by the gene. Such compounds are used as pharmaceuticals for treating or preventing lung cancer and esophageal cancer or detecting agents for diagnosing lung cancer and esophageal cancer and assessing a prognosis of lung cancer and/or esophageal cancer patient.

Specifically, the present invention provides the method of screening for an agent or compound useful in diagnosing, treating or preventing cancers using the TBC1D7 polypeptide. An embodiment of this screening method includes the steps of:

(a) contacting a test agent or compound with a polypeptide selected from the group consisting of TBC1D7 protein, or fragment thereof;

(b) detecting binding between the polypeptide and said test agent or compound;

(c) selecting the test agent or compound that binds to said polypeptides of step (a).

According to the present invention, the therapeutic effect of a candidate agent or compound on suppressing the binding to TBC1D7 protein, or a candidate agent or compound for treating or preventing cancer relating to TBC1D7 (e.g., lung and esophageal cancers) may be evaluated. Therefore, the present invention also provides a method of screening for a candidate agent or compound for suppressing cell proliferation, or a candidate agent or compound for treating or preventing cancer (e.g., lung and esophageal cancers), using the TBC1D7 polypeptide or fragments thereof including the steps of:

a) contacting a test agent or compound with the TBC1D7 polypeptide or a functional fragment thereof; and

b) detecting the binding between the polypeptide and the test agent or compound, and

c) correlating the binding of b) with the therapeutic effect of the test agent or compound.

In the present invention, the therapeutic effect may be correlated with the binding properties of the test agent or compound towards the TBC1D7 polypeptide (or fragment thereof). For example, when the test agent or compound binds to the TBC1D7 polypeptide (or fragment thereof), the test agent or compound may identified or selected as the candidate agent or compound having the therapeutic effect. Alternatively, when the test agent or compound does not bind to the TBC1D7 polypeptide (or fragment thereof), the test agent or compound may identified as the agent or compound having no significant therapeutic effect.

The method of the present invention will be described in more detail below.

The TBC1D7 polypeptide to be used for screening can be a recombinant polypeptide or a protein derived from the nature or a partial peptide thereof. The polypeptide to be contacted with a test compound can be, for example, a purified polypeptide, a soluble protein, a form bound to a carrier or a fusion protein fused with other polypeptides.

As a method of screening for proteins, for example, that bind to TBC1D7 polypeptide using TBC1D7 polypeptide, many methods well known by a person skilled in the art can be used. Such a screening can be conducted by, for example, immunoprecipitation method. The gene encoding TBC1D7 polypeptide is expressed in host (e.g., animal) cells and so on by inserting the gene to an expression vector for foreign genes, for example, pSV2neo, pcDNA I, pcDNA3.1, pCAGGS and pCD8.

The promoter to be used for the expression can be any promoter that can be used commonly and include, for example, the SV40 early promoter (Rigby in Williamson (ed.), Genetic Engineering, vol. 3. Academic Press, London, 83-141 (1982)), the EF-alpha promoter (Kim et al., Gene 91: 217-23 (1990)), the CAG promoter (Niwa et al., Gene 108: 193 (1991)), the RSV LTR promoter (Cullen, Methods in Enzymology 152: 684-704 (1987)) the SR alpha promoter (Takebe et al., Mol Cell Biol 8: 466 (1988)), the CMV immediate early promoter (Seed and Aruffo, Proc Natl Acad Sci USA 84: 3365-9 (1987)), the SV40 late promoter (Gheysen and Fiers, J Mol Appl Genet 1: 385-94 (1982)), the Adenovirus late promoter (Kaufman et al., Mol Cell Biol 9: 946 (1989)), the HSV TK promoter and so on.

The introduction of the gene into host cells to express a foreign gene can be performed according to any methods, for example, the electroporation method (Chu et al., Nucleic Acids Res 15: 1311-26 (1987)), the calcium phosphate method (Chen and Okayama, Mol Cell Biol 7: 2745-52 (1987)), the DEAE dextran method (Lopata et al., Nucleic Acids Res 12: 5707-17 (1984); Sussman and Milman, Mol Cell Biol 4: 1641-3 (1984)), the Lipofectin method (Derijard B., Cell 76: 1025-37 (1994); Lamb et al., Nature Genetics 5: 22-30 (1993): Rabindran et al., Science 259: 230-4 (1993)) and so on.

The polypeptide encoded by TBC1D7 gene can be expressed as a fusion protein including a recognition site (epitope) of a monoclonal antibody by introducing the epitope of the monoclonal antibody, whose specificity has been revealed, to the N- or C-terminus of the polypeptide. A commercially available epitope-antibody system can be used (Experimental Medicine 13: 85-90 (1995)). Vectors which can express a fusion protein with, for example, b-galactosidase, maltose binding protein, glutathione S-transferase, green florescence protein (GFP) and so on by the use of its multiple cloning sites are commercially available. Also, a fusion protein prepared by introducing only small epitopes consisting of several to a dozen amino acids so as not to change the property of the TBC1D7 polypeptide by the fusion is also reported. Epitopes, for example, polyhistidine (His-tag), influenza aggregate HA, human c-myc, FLAG, Vesicular stomatitis virus glycoprotein (VSV-GP), T7 gene 10 protein (T7-tag), human simple herpes virus glycoprotein (HSV-tag), E-tag (an epitope on monoclonal phage) and such, and monoclonal antibodies recognizing them can be used as the epitope-antibody system for screening proteins binding to the TBC1D7 polypeptide (Experimental Medicine 13: 85-90 (1995)).

In immunoprecipitation, an immune complex is formed by adding these antibodies to cell lysate prepared using an appropriate detergent. The immune complex consists of the TBC1D7 polypeptide, a polypeptide including the binding ability with the polypeptide, and an antibody. Immunoprecipitation can be also conducted using antibodies against the TBC1D7 polypeptide, besides using antibodies against the above epitopes, which antibodies can be prepared as described above. An immune complex can be precipitated, for example by Protein A sepharose or Protein G sepharose when the antibody is a mouse IgG antibody. If the polypeptide encoded by TBC1D7 gene is prepared as a fusion protein with an epitope, for example, GST, an immune complex can be formed in the same manner as in the use of the antibody against the TBC1D7 polypeptide, using a substance specifically binding to these epitopes, for example, glutathione-Sepharose 4B.

Immunoprecipitation can be performed by following or according to, for example, the methods in the literature (Harlow and Lane, Antibodies, 511-52, Cold Spring Harbor Laboratory publications, New York (1988)).

SDS-PAGE is commonly used for analysis of immunoprecipitated proteins and the bound protein can be analyzed by the molecular weight of the protein using gels with an appropriate concentration. Since the protein bound to TBC1D7 polypeptide is difficult to detect by a common staining method, for example, Coomassie staining or silver staining, the detection sensitivity for the protein can be improved by culturing cells in culture medium containing radioactive isotope, “S-methionine or “S-cystein, labeling proteins in the cells, and detecting the proteins. The target protein can be purified directly from the SDS-polyacrylamide gel and its sequence can be determined, when the molecular weight of a protein has been revealed.

As a method of screening for proteins binding to TBC1D7 polypeptide using the polypeptide, for example, West-Western blotting analysis (Skolnik et al., Cell 65: 83-90 (1991)) can be used. Specifically, a protein binding to the TBC1D7 polypeptide can be obtained by preparing a cDNA library from cultured cells (e.g., lung cancer cell line or esophageal cancer cell line) expected to express a protein binding to the TBC1D7 polypeptide using a phage vector (e.g., ZAP), expressing the protein on LB-agarose, fixing the protein expressed on a filter, reacting the purified and labeled TBC1D7 polypeptide with the above filter, and detecting the plaques expressing proteins bound to TBC1D7 polypeptide according to the label. The polypeptide of the invention can be labeled by utilizing the binding between biotin and avidin, or by utilizing an antibody that specifically binds to TBC1D7 polypeptide, or a peptide or polypeptide (for example, GST) that is fused to TBC1D7 polypeptide. Methods using radioisotope or fluorescence and such can be also used.

Alternatively, in another embodiment of the screening method of the present invention, a two-hybrid system utilizing cells can be used (”MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton and Treisman, Cell 68: 597-612 (1992)”, “Fields and Sternglanz, Trends Genet 10: 286-92 (1994)”).

In the two-hybrid system, the polypeptide of the invention is fused to the SRF-binding region or GAL4-binding region and expressed in yeast cells. A cDNA library is prepared from cells expected to express a protein binding to the polypeptide of the invention, such that the library, when expressed, is fused to the VP16 or GAL4 transcriptional activation region. The cDNA library is then introduced into the above yeast cells and the cDNA derived from the library is isolated from the positive clones detected (when a protein binding to the polypeptide of the invention is expressed in yeast cells, the binding of the two activates a reporter gene, making positive clones detectable). A protein encoded by the cDNA can be prepared by introducing the cDNA isolated above to E. coli and expressing the protein. As a reporter gene, for example, Ade2 gene, lacZ gene, CAT gene, luciferase gene and such can be used in addition to the HIS3 gene.

A compound binding to the polypeptide encoded by TBC1D7 gene can also be screened using affinity chromatography. For example, the polypeptide of the invention can be immobilized on a carrier of an affinity column, and a test compound, containing a protein capable of binding to the polypeptide of the invention, is applied to the column. A test compound herein can be, for example, cell extracts, cell lysates, etc. After loading the test compound, the column is washed, and compounds bound to the polypeptide of the invention can be prepared. When the test compound is a protein, the amino acid sequence of the obtained protein is analyzed, an oligo DNA is synthesized based on the sequence, and cDNA libraries are screened using the oligo DNA as a probe to obtain a DNA encoding the protein.

A biosensor using the surface plasmon resonance phenomenon can be used as a mean for detecting or quantifying the bound compound in the present invention. When such a biosensor is used, the interaction between the polypeptide of the invention and a test compound can be observed real-time as a surface plasmon resonance signal, using only a minute amount of polypeptide and without labeling (for example, BIAcore, Pharmacia). Therefore, it is possible to evaluate the binding between the polypeptide of the invention and a test compound using a biosensor for example, BIAcore.

The methods of screening for molecules that bind when the immobilized TBC1D7 polypeptide is exposed to synthetic chemical compounds, or natural substance banks or a random phage peptide display library, and the methods of screening using high-throughput based on combinatorial chemistry techniques (Wrighton et al., Science 273: 458-64 (1996); Verdine, Nature 384: 11-13 (1996); Hogan, Nature 384: 17-9 (1996)) to isolate not only proteins but chemical compounds that bind to the TBC1D7 protein (including agonist and antagonist) are well known to one skilled in the art.

(iii) Screening for Compound that Suppress the Biological Activity of TBC1D7 Gene

In the present invention, the TBC1D7 protein has the activity of promoting cell proliferation of cancer cells (FIG. 3C) and invasion activity (FIG. 3D). Using this biological activity, a compound which inhibits this activity of this protein can be screened. Therefore, the present invention provides a method of screening for a compound for treating or preventing cancers expressing TBC1D7 gene, e.g. lung cancers (non-small cell lung cancer or small cell lung cancer) or esophageal cancer, using the polypeptide encoded by TBC1D7 gene.

Specifically, the present invention provides the following methods of [1] to [19]:

[1] A method of screening for an agent or compound useful in treating or preventing cancers expressing TBC1D7, said method including the steps of:

(a) contacting a test agent or compound with a cell expressing a polynucleotide encoding a polypeptide encoded by the gene expressing in cancer, or functional equivalent thereof;

(b) detecting a level of said polynucleotide or polypeptide of step (a);

(c) comparing said level detected in the step (b) with those detected in the absence of the test agent or compound; and

(d) selecting the test agent or compound that reduce or inhibit said level of (c).

[2] The method of [1], wherein said level is detected by any one of the method select from the group consisting of:

(a) detecting the amount of the mRNA encoding the TBC1D7 polypeptide, or functional equivalent thereof;

(b) detecting the amount of the TBC1D7 polypeptide or functional equivalent thereof; and

(c) detecting the biological activity of the TBC1D7 polypeptide or functional equivalent thereof.

[3] The method of [2], wherein the biological activity is any one of the activity select from the group consisting of:

(a) a proliferation activity of the cell expressing a polypeptide selected from the group consisting of TBC1D7 polypeptide, or functional equivalent thereof; and

(b) an invasion activity of the cell expressing an TBC1D7 polypeptide or functional equivalent thereof.

According to the present invention, the therapeutic effect of a candidate agent or compound on suppressing the level or biological activity (e.g., cell proliferation-promoting activity) of TBC1D7, or a candidate agent or compound for treating or preventing cancer relating to TBC1D7 (e.g., lung and esophageal cancers) may be evaluated. Therefore, the present invention also provides a method of screening for a candidate agent or compound for suppressing cell proliferation, or a candidate agent or compound for treating or preventing cancer (e.g., lung and esophageal cancers), using TBC1D7 including the steps of:

(a) contacting a test agent or compound with a cell expressing a polynucleotide encoding a polypeptide encoded by the gene expressing in cancer, or functional equivalent thereof;

(b) detecting a level or biological activity of said polynucleotide or polypeptide of step (a); and

(c) correlating the level or biological activity of b) with the therapeutic effect of the test agent or compound.

In the present invention, the therapeutic effect may be correlated with the level or biological activity of the TBC1D7 polypeptide or polynucleotide (or a functional fragment thereof). For example, when the test agent or compound suppresses or inhibits the level or biological activity of TBC1D7 as compared to a level or biological activity detected in the absence of the test agent or compound, the test agent or compound may identified or selected as the candidate agent or compound having the therapeutic effect. Alternatively, when the test agent or compound does not suppress or inhibit the level or biological activity of TBC1D7 as compared to a level or biological activity detected in the absence of the test agent or compound, the test agent or compound may identified as the agent or compound having no significant therapeutic effect.

The method of the present invention will be described in more detail below.

Any polypeptides can be used for screening so long as they include the biological activity of the TBC1D7 protein. Such biological activity includes the cell-proliferating activity, the activity of promoting cell invasion or the RAB 17, 14-3-3 zeta or TSC1-binding activity. For example, TBC1D7 protein can be used and polypeptides functionally equivalent to these proteins can also be used. Such polypeptides can be expressed endogenously or exogenously by cells.

The compound isolated by this screening is a candidate for antagonists of the polypeptide encoded by TBC1D7 gene. The term “antagonist” refers to molecules that inhibit the function of the polypeptide by binding thereto. Said term also refers to molecules that reduce or inhibit expression of the gene encoding TBC1D7. Moreover, a compound isolated by this screening is a candidate for compounds which inhibit the in vivo interaction of the TBC1D7 polypeptide with molecules (including DNAs and proteins).

When the biological activity to be detected in the present method is cell proliferation, it can be detected, for example, by preparing cells which express the polypeptide selected from the group consisting of TBC1D7, culturing the cells in the presence of a test compound, and determining the speed of cell proliferation, measuring the cell cycle and such, as well as by measuring the colony formation activity, e.g. MTT assay, colony formation assay or FACS shown in [EXAMPLE 1-j].

The term of “suppress the biological activity” as defined herein refers to at least 10% suppression of the biological activity of TBC1D7 in comparison with in absence of the compound, for example, at least 25%, 50% or 75% suppression, for example, at least 90% suppression.

(iv) Screening for Compounds that Alter the Expression of TBC1D7

In the present invention, the decrease of the expression of TBC1D7 by a double-stranded molecule specific for TBC1D7 causes inhibiting cancer cell proliferation (FIG. 3). Therefore, compounds that can be used in the treatment or prevention of cancer can be identified through screenings that use the expression levels of TBC1D7 as indices. In the context of the present invention, such screening can include, for example, the following steps:

(a) contacting a candidate compound with a cell expressing TBC1D7;

(b) detecting the expression level of TBC1D7; and

(c) selecting the candidate compound that reduces the expression level of TBC1D7 as compared to a control.

According to the present invention, the therapeutic effect of a candidate compound on altering the expression of TBC1D7, or a candidate compound for treating or preventing cancer relating to TBC1D7 (e.g., lung and esophageal cancers) may be evaluated. Therefore, the present invention also provides a method of screening for a candidate compound for altering the expression of TBC1D7, or a candidate compound for treating or preventing cancer (e.g., lung and esophageal cancers) including the steps of:

a) contacting a candidate compound with a cell expressing TBC1D7;

b) detecting the expression level of TBC1D7; and

c) correlating the expression level of b) with the therapeutic effect of the test compound.

In the present invention, the therapeutic effect may be correlated with the expression level of TBC1D7. For example, when the test compound suppresses the expression level of TBC1D7 as compared to a level detected in the absence of the test compound, the test compound may identified or selected as the candidate compound having the therapeutic effect. Alternatively, when the test compound does not suppress the expression level of TBC1D7 as compared to a level detected in the absence of the test compound, the test compound may identified as the agent or compound having no significant therapeutic effect.

The method of the present invention will be described in more detail below.

Cells expressing the TBC1D7 include, for example, cell lines established from lung cancer or esophageal cancer; such cells can be used for the above screening of the present invention (e.g., A549 and LC319). The expression level can be estimated by methods well known to one skilled in the art, for example, RT-PCR, Northern bolt assay, Western bolt assay, immunostaining, ELISA or flow cytometry analysis. The term of “reduce the expression level” as defined herein refers to at least 10% reduction of expression level of TBC1D7 in comparison to the expression level in absence of the compound, for example, at least 25%, 50% or 75% reduced level, for example, at least 95% reduced level. The compound herein includes chemical compound, double-strand nucleotide, and so on. The preparation of the double-strand nucleotide is in aforementioned description. In the method of screening, a compound that reduces the expression level of TBC1D7 can be selected as candidate agents or compounds to be used for the treatment or prevention of cancers, e.g. lung cancer and/or esophageal cancer.

Alternatively, the screening method of the present invention can include the following steps:

(a) contacting a candidate compound with a cell into which a vector, including the transcriptional regulatory region of TBC1D7 and a reporter gene that is expressed under the control of the transcriptional regulatory region, has been introduced;

(b) measuring the expression or activity of said reporter gene; and

(c) selecting the candidate compound that reduces the expression or activity of said reporter gene.

Suitable reporter genes and host cells are well known in the art. For example, reporter genes are luciferase, green florescence protein (GFP), Discosoma sp. Red Fluorescent Protein (DsRed), Chrolamphenicol Acetyltransferase (CAT), lacZ and beta-glucuronidase (GUS), and host cell is COS7, HEK293, HeLa and so on. The reporter construct required for the screening can be prepared by connecting reporter gene sequence to the transcriptional regulatory region of CX. The transcriptional regulatory region of CX herein is the region from start codon to at least 500 bp upstream, for example, 1000 bp, for example, 5000 or 10000 bp upstream, but not restricted. A nucleotide segment containing the transcriptional regulatory region can be isolated from a genome library or can be propagated by PCR. Methods for identifying a transcriptional regulatory region, and also assay protocol are well known (Molecular Cloning third edition chapter 17, 2001, Cold Springs Harbor Laboratory Press).

The vector containing the said reporter construct is infected to host cells and the expression or activity of the reporter gene is detected by method well known in the art (e.g., using luminometer, absorption spectrometer, flow cytometer and so on). “Reduces the expression or activity” as defined herein refers to at least 10% reduction of the expression or activity of the reporter gene in comparison with in absence of the compound, for example, at least 25%, 50% or 75% reduction, for example, at least 95% reduction.

Aspects of the present invention are described in the following examples, which are not intended to limit the scope of the invention described in the claims.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

The invention will be further described in the following examples, which do not limit the scope of the invention described in the claims.

(v) Screening Using the Binding of TBC1D7 and RAB17, 14-3-3 Zeta or TSC1 as an Index

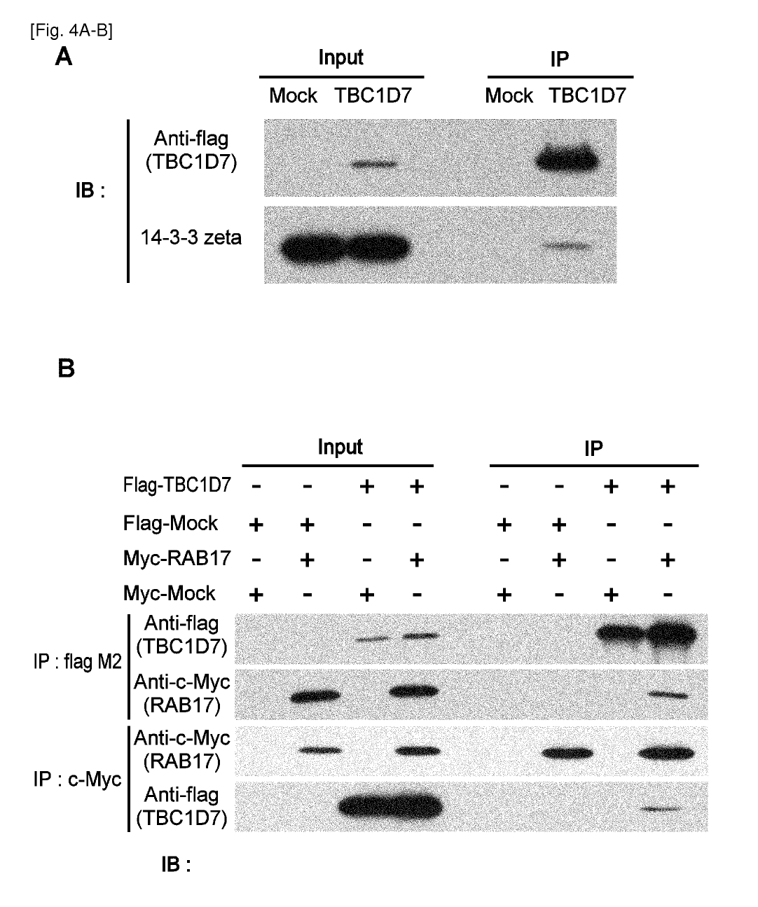

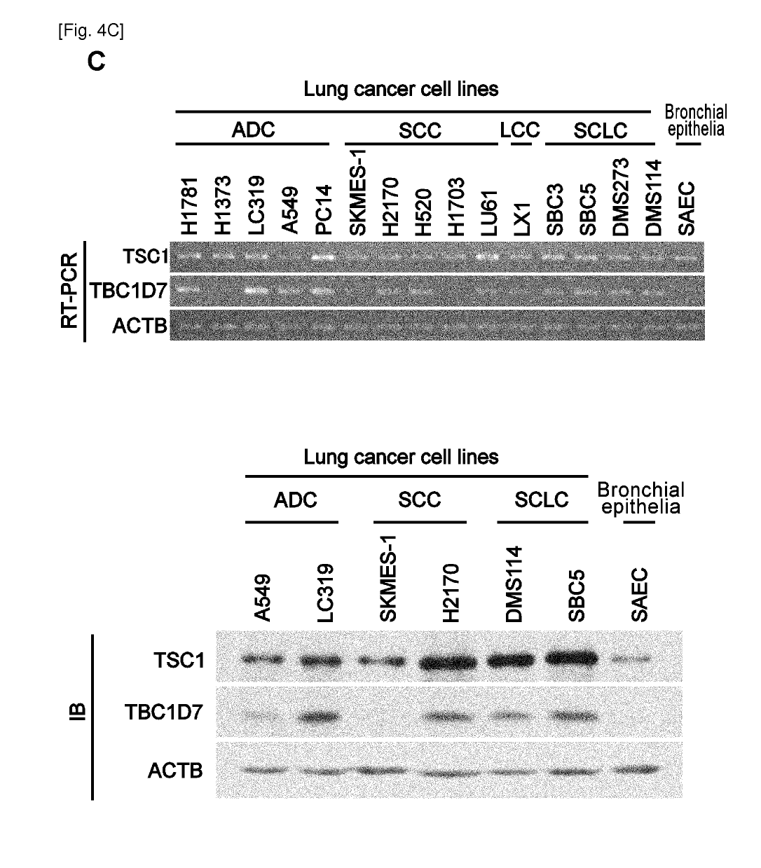

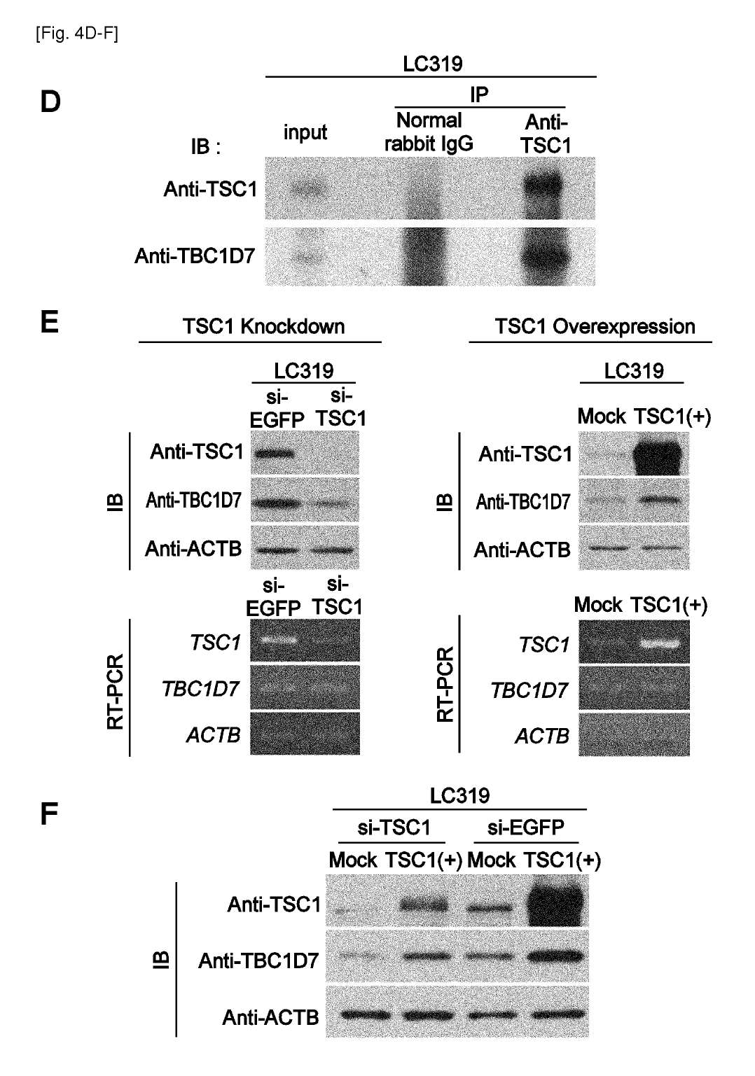

In the present invention, it was confirmed that the TBC1D7 protein interacts with RAB17, 14-3-3 zeta or TSC1 protein (FIG. 4). Thus, a compound that inhibits the binding between TBC1D7 protein and RAB17, 14-3-3 zeta or TSC1 protein can be screened using such a binding of TBC1D7 protein and RAB17, 14-3-3 zeta or TSC1 protein as an index. Therefore, the present invention provides a method for screening a compound for inhibiting the binding between TBC1D7 protein and RAB 17, 14-3-3 zeta or TSC1 protein can be screened using such a binding of TBC1D7 protein and RAB17, 14-3-3 zeta or TSC1 protein as an index. Furthermore, the present invention also provides a method for screening a compound for inhibiting or reducing a growth of cancer cells expressing TBC1D7, e.g. lung cancer cell and/or esophageal cancer cell, and a compound for treating or preventing cancers, e.g. lung cancer and/or esophageal cancer.

Specifically, the present invention provides the following methods of [1] to [5]:

[1] A method of screening for an agent or compound that interrupts a binding between a TBC1D7 polypeptide and a RAB17, 14-3-3 zeta or TSC1 polypeptide, said method including the steps of:

(a) contacting a TBC1D7 polypeptide or functional equivalent thereof with a RAB 17, 14-3-3 zeta or TSC1 polypeptide or functional equivalent thereof in the presence of a test agent or compound;

(b) detecting a binding between the polypeptides;

(c) comparing the binding level detected in the step (b) with those detected in the absence of the test agent or compound; and

(d) selecting the test agent or compound that reduce or inhibits the binding level.

[2] A method of screening for an agent or compound useful in treating or preventing cancers, said method including the steps of:

(a) contacting a TBC1D7 polypeptide or functional equivalent thereof with a RAB17, 14-3-3 zeta or TSC1 polypeptide or functional equivalent thereof in the presence of a test agent or compound;

(b) detecting a binding between the polypeptides;

(c) comparing the binding level detected in the step (b) with those detected in the absence of the test agent or compound; and

(d) selecting the test agent or compound that reduce or inhibits the binding level.

[3] The method of [1] or [2], wherein the functional equivalent of TBC1D7 including the RAB17, 14-3-3 zeta or TSC1-binding domain.

[4] The method of [1] or [2], wherein the functional equivalent of RAB 17, 14-3-3 zeta or TSC1 including the TBC1D7-binding domain.

[5] The method of [1], wherein the cancer is selected from the group consisting of lung cancers and esophageal cancer.

In the context of the present invention, a functional equivalent of an TBC1D7, RAB17, 14-3-3 zeta or TSC1 polypeptide is a polypeptide that has a biological activity equivalent to a TBC1D7 polypeptide (SEQ ID NO: 2), RAB17 (SEQ ID NO: 12), 14-3-3 zeta (SEQ ID NO: 14) or TSC1 (SEQ ID NO: 45) polypeptide, respectively (see, (1) Cancer-related genes and cancer-related protein, and functional equivalent thereof in Definition). Particularly, the functional equivalent of TBC1D7 is a poly peptide fragment containing the binding domain to RAB17, 14-3-3 zeta or TSC1, such as amino acid sequence of SEQ ID NO: 28. More specifically, the functional equivalent of RAB 17 is a polypeptide fragment of SEQ ID NO: 12 and of 14-3-3 zeta is a polypeptide fragment of SEQ ID NO: 14 and of TSC1 is a fragment of SEQ ID NO: 45 including the TBC1D7-binding domain.

As a method of screening for compounds that modulates, e.g. inhibits, the binding of TBC1D7 to RAB17, 14-3-3 zeta or TSC1, many methods well known by one skilled in the art can be used.

A polypeptide to be used for screening can be a recombinant polypeptide or a protein derived from natural sources, or a partial peptide thereof. Any test compound aforementioned can be used for screening.

As a method of screening for proteins, for example, that bind to a polypeptide using TBC1D7, RAB17, 14-3-3 zeta or TSC1 polypeptide or functionally equivalent thereof (see, (1) Cancer-related genes and cancer-related protein, and functional equivalent thereof in Definition), many methods well known by a person skilled in the art can be used. Such a screening can be conducted using, for example, an immunoprecipitation, West-Western blotting analysis (Skolnik et al., Cell 65: 83-90 (1991)), a two-hybrid system utilizing cells (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton and Treisman, Cell 68: 597-612 (1992)”, “Fields and Sternglanz, Trends Genet 10: 286-92 (1994)”), affinity chromatography and A biosensor using the surface plasmon resonance phenomenon (see (i) General screening Method). Any aforementioned test compound can be used (see (1) Test compounds for screening).

In some embodiments, this method further includes the step of detecting the binding of the candidate compound to TBC1D7 protein, RAB17 protein, 14-3-3 zeta protein or TSC1 protein, or detecting the level of binding TBC1D7 protein to RAB17, 14-3-3 zeta protein or TSC1 protein. Cells expressing TBC1D7 protein, RAB17 and TSC1 protein and/or 14-3-3 zeta proteins include, for example, cell lines established from cancer, e.g. lung cancer and/or esophageal cancer, such cells can be used for the above screening of the present invention so long as the cells express these two genes. Alternatively cells can be transfected both or either of expression vectors of TBC1D7 and RAB17, 14-3-3 zeta and/or TSC1 protein, so as to express these two genes. The binding of TBC1D7 protein to RAB17, 14-3-3 zeta protein and/or TSC1 protein can be detected by immunoprecipitation assay using an anti-TBC1D7 antibody, anti-RAB17 antibody anti-14-3-3 zeta antibody and TSC1 antibody (FIG. 4).

According to the present invention, the therapeutic effect of a candidate agent or compound on interrupting the binding between a TBC1D7 polypeptide and a RAB 17, 14-3-3 zeta, or TSC1 polypeptide, or a candidate agent or compound for treating or preventing cancer relating to TBC1D7 (e.g., lung and esophageal cancers) may be evaluated. Therefore, the present invention also provides a method of screening for a candidate agent or compound for interrupting the binding between a TBC1D7 polypeptide and a RAB 17, 14-3-3 zeta, or TSC1 polypeptide, or a candidate agent or compound for treating or preventing cancer (e.g., lung and esophageal cancers), using a TBC1D7 polypeptide or functional equivalent thereof including the steps of:

(a) contacting a TBC1D7 polypeptide or functional equivalent thereof with a RAB 17, 14-3-3 zeta or TSC1 polypeptide or functional equivalent thereof in the presence of a test agent or compound;

(b) detecting a binding between the polypeptides;

(c) comparing the binding level detected in the step (b) with those detected in the absence of the test agent or compound; and

(d) correlating the binding level of (c) with the therapeutic effect of the test agent or compound;

In the present invention, the therapeutic effect may be correlated with the binding between a TBC1D7 polypeptide and a RAB17, 14-3-3 zeta, or TSC1 polypeptide. For example, when the test agent or compound suppresses or inhibits the level of binding between the polypeptides as compared to a level detected in the absence of the test agent or compound, the test agent or compound may identified or selected as the candidate agent or compound having the therapeutic effect. Alternatively, when the test agent or compound does not suppress or inhibit the level of binding between the polypeptides as compared to a level detected in the absence of the test agent or compound, the test agent or compound may identified as the agent or compound having no significant therapeutic effect.

Dominant Negative Protein that Inhibits Interaction of TBC1D7

The present invention relates to inhibitory polypeptides that contain YWITRRFVNQLNTKYRDSLP (SEQ ID NO.: 28). In some preferred embodiments, the inhibitory polypeptide includes YWITRRFVNQLNTKYRDSLP (SEQ ID NO.: 28); a polypeptide functionally equivalent to the polypeptide; or polynucleotide encoding those polypeptides, wherein the polypeptide lacks the kinase activity of TTK for EGFR. It is a novel finding proved by the present invention that that EGFR fragment inhibits the lung cancer cell proliferation.

The polypeptides including the selected amino acid sequence of the present invention, can be of any length, so long as the polypeptide contain the amino acid sequence of YWITRRFVNQLNTKYRDSLP (SEQ ID NO.: 28) and inhibits cancer cell proliferation. In some embodiments, the polypeptides are truncated forms of TBC1D7 consisiting of less than about 250 amino acids from SEQ ID NO 2. In some embodiments the polypeptides may consist of less than about 100 amino acids. In some embodiments, the length of the amino acid sequence may range from 20 to 60 residues.

The polypeptides of the present invention may contain two or more “selected amino acid sequences”. The two or more “selected amino acid sequences” may be the same or different amino acid sequences. Furthermore, the “selected amino acid sequences” can be linked directly. Alternatively, they may be disposed with any intervening sequences among them.

Furthermore, the present invention relates to polypeptides homologous (i.e., share sequence identity) to the YWITRRFVNQLNTKYRDSLP (SEQ ID NO.: 28) polypeptide specifically disclosed here. In the present invention, polypeptides homologous to the YWITRRFVNQLNTKYRDSLP (SEQ ID NO.: 28) polypeptide are those which contain any mutations selected from addition, deletion, substitution and insertion of one or several amino acid residues and are functionally equivalent. The phrase “functionally equivalent” refers to having the function to inhibit the interaction between TBC1D7 and otherprotein sach as TSC1 and inhibit the cell proliferation. Therefore, polypeptides functionally equivalent to the YWITRRFVNQLNTKYRDSLP (SEQ ID NO.: 28) peptide in the present invention preferably have amino acid mutations in sites other than the YWITRRFVNQLNTKYRDSLP (SEQ ID NO.: 28) sequence. Amino acid sequences of polypeptides functionally equivalent to the YWITRRFVNQLNTKYRDSLP (SEQ ID NO.:28) peptide in the present invention conserve the YWITRRFVNQLNTKYRDSLP (SEQ ID NO.:28) sequence, and have 60% or higher, usually 70% or higher, preferably 80% or higher, more preferably 90% or higher, or 95% or higher, and further more preferably 98% or higher homology to a “selected amino acid sequence”. Amino acid sequence homology can be determined using algorithms well known in the art, for example, BLAST or ALIGN set to their default settings.

The polypeptides of the present invention can be chemically synthesized from any position based on selected amino acid sequences. Methods used in the ordinary peptide chemistry can be used for the method of synthesizing polypeptides. Specifically, the methods include those described in the following documents and Japanese Patent publications

Peptide Synthesis, Interscience, New York, 1966; The Proteins, Vol. 2, Academic Press Inc., New York, 1976;

Peputido gousei (Peptide Synthesis), Maruzen (Inc.), 1975;

Peputido gousei no kiso to jikken (Fundamental and Experimental Peptide Synthesis), Maruzen (Inc.), 1985;

Iyakuhin no kaihatsu (Development of Pharmaceuticals), Sequel, Vol. 14: Peputido gousei (Peptide Synthesis), Hirokawa Shoten, 1991;

International Patent Publication WO99/67288.

The polypeptides of the present invention can be also synthesized by known genetic engineering techniques. An example of genetic engineering techniques is as follows. Specifically, DNA encoding a desired peptide is introduced into an appropriate host cell to prepare a transformed cell. The polypeptides of the present invention can be obtained by recovering polypeptides produced by this transformed cell. Alternatively, a desired polypeptide can be synthesized with an in vitro translation system, in which necessary elements for protein synthesis are reconstituted in vitro.

When genetic engineering techniques are used, the polypeptide of the present invention can be expressed as a fused protein with a peptide having a different amino acid sequence. A vector expressing a desired fusion protein can be obtained by linking a polynucleotide encoding the polypeptide of the present invention to a polynucleotide encoding a different peptide so that they are in the same reading frame, and then intorducing the resulting nucleotide into an expression vector. The fusion protein is expressed by transforming an appropriate host with the resulting vector. Different peptides to be used in forming fusion proteins include the following peptides:
FLAG (Hopp et al., (1988) BioTechnology 6, 1204-10),
6× His consisting of six His (histidine) residues, 10× His,
Influenza hemagglutinin (HA),
Human c-myc fragment,
VSV-GP fragment,
p18 HIV fragment,
T7-tag,
HSV-tag,
E-tag,
SV40T antigen fragment,
lck tag,
alpha-tubulin fragment,
B-tag,
Protein C fragment,
GST (glutathione-S-transferase),
HA (Influenza hemagglutinin),
Immunoglobulin constant region,
beta-galactosidase, and
MBP (maltose-binding protein).

The polypeptide of the present invention can be obtained by treating the fusion protein thus produced with an appropriate protease, and then recovering the desired polypeptide. To purify the polypeptide, the fusion protein is captured in advance with affinity chromatography that binds with the fusion protein, and then the captured fusion protein can be treated with a protease. With the protease treatment, the desired polypeptide is separated from affinity chromatography, and the desired polypeptide with high purity is recovered.

The polypeptides of the present invention include modified polypeptides. In the present invention, the term “modified” refers, for example, to binding with other substances. Accordingly, in the present invention, the polypeptides of the present invention may further include other substances such as cell-membrane permeable substance. The other substances include organic compounds such as peptides, lipids, saccharides, and various naturally-occurring or synthetic polymers. The polypeptides of the present invention may have any modifications so long as the polypeptides retain the desired activity of inhibiting the interaction of TBC1D7. In some embodiments, the inhibitory polypeptides can directly compete with TSC1 binding to TBC1D7. Modifications can also confer additive functions on the polypeptides of the invention. Examples of the additive functions include targetability, deliverability, and stabilization.

Preferred examples of modifications in the present invention include, for example, the introduction of a cell-membrane permeable substance. Usually, the intracellular structure is cut off from the outside by the cell membrane. Therefore, it is difficult to efficiently introduce an extracellular substance into cells. Cell membrane permeability can be conferred on the polypeptides of the present invention by modifying the polypeptides with a cell-membrane permeable substance. As a result, by contacting the polypeptide of the present invention with a cell, the polypeptide can be delivered into the cell to act thereon.

The “cell-membrane permeable substance” refers to a substance capable of penetrating the mammalian cell membrane to enter the cytoplasm. For example, a certain liposome fuses with the cell membrane to release the content into the cell. Meanwhile, a certain type of polypeptide penetrates the cytoplasmic membrane of mammalian cell to enter the inside of the cell. For polypeptides having such a cell-entering activity, cytoplasmic membranes and such in the present invention are preferable as the substance. Specifically, the present invention includes polypeptides having the following general formula.

[R]-[D];

wherein,

[R] represents a cell-membrane permeable substance; [D] represents a fragment sequence containing YWITRRFVNQLNTKYRDSLP (SEQ ID NO.:28). In the above-described general formula, [R] and [D] can be linked directly or indirectly through a linker. Peptides, compounds having multiple functional groups, or such can be used as a linker. Specifically, amino acid sequences containing -GGG- can be used as a linker. Alternatively, a cell-membrane permeable substance and a polypeptide containing a selected sequence can be bound to the surface of a minute particle. [R] can be linked to any positions of [D]. Specifically, [R] can be linked to the N terminal or C terminal of [D], or to a side chain of amino acids constituting [D]. Furthermore, more than one [R] molecule can be linked to one molecule of [D]. The [R] molecules can be introduced to different positions on the [D] molecule. Alternatively, [D] can be modified with a number of [R]s linked together.

For example, there have been reported a variety of naturally-occurring or artificially synthesized polypeptides having cell-membrane permeability (Joliot A. & Prochiantz A., Nat Cell Biol. 2004; 6: 189-96). All of these known cell-membrane permeable substances can be used for modifying polypeptides in the present invention. In the present invention, for example, any substance selected from the following group can be used as the above-described cell-permeable substance:
poly-arginine; Matsushita et al., (2003) J. Neurosci.; 21, 6000-7.
[Tat/RKKRRQRRR] (SEQ ID NO: 29) Frankel et al., (1988) Cell 55,1189-93.
Green & Loewenstein (1988) Cell 55, 1179-88.
[Penetratin/RQIKIWFQNRRMKWKK] (SEQ ID NO: 30)
Derossi et al., (1994) J. Biol. Chem. 269, 10444-50.
[Buforin II/TRSSRAGLQFPVGRVHRLLRK] (SEQ ID NO: 31)
Park et al., (2000) Proc. Natl Acad. Sci. USA 97, 8245-50.
[Transportan/GWTLNSAGYLLGKINLKALAALAKKIL] (SEQ ID NO: 32)
Pooga et al., (1998) FASEB J. 12, 67-77.
[MAP (model amphipathic peptide)/KLALKLALKALKAALKLA] (SEQ ID NO: 33)
Oehlke et al., (1998) Biochim. Biophys. Acta. 1414, 127-39.
[K-FGF/AAVALLPAVLLALLAP] (SEQ ID NO: 34)
Lin et al., (1995) J. Biol. Chem. 270, 14255-8.
[Ku70/VPMLK] (SEQ ID NO: 35)
Sawada et al., (2003) Nature Cell Biol. 5, 352-7.
[Ku70/PMLKE] (SEQ ID NO: 36)
Sawada et al., (2003) Nature Cell Biol. 5, 352-7.
[Prion/MANLGYWLLALFVTMWTDVGLCKKRPKP] (SEQ ID NO: 37)
Lundberg et al., (2002) Biochem. Biophys. Res. Commun. 299, 85-90.
[pVEC/LLIILRRRIRKQAHAHSK] (SEQ ID NO: 38)
Elmquist et al., (2001) Exp. Cell Res. 269, 237-44.
[Pep-1/KETWWETWWTEWSQPKKKRKV] (SEQ ID NO: 39)
Morris et al., (2001) Nature Biotechnol. 19, 1173-6.
[SynB1/RGGRLSYSRRRFSTSTGR] (SEQ ID NO: 40)
Rousselle et al., (2000) Mol. Pharmacol. 57, 679-86.
[Pep-7/SDLWEMMMVSLACQY] (SEQ ID NO: 41)
Gao et al., (2002) Bioorg. Med. Chem. 10, 4057-65.
[HN-1/TSPLNIHNGQKL] (SEQ ID NO: 42)
Hong & Clayman (2000) Cancer Res. 60, 6551-6.

In the present invention, the poly-arginine, which is listed above as an example of cell-membrane permeable substances, is constituted by any number of arginine residues. Specifically, for example, it is constituted by consecutive 5-20 arginine residues. The preferable number of arginine residues is 11 (SEQ ID NO: 43).

Pharmaceutical compositions including YWITRRFVNQLNTKYRDSLP (SEQ ID NO.: 28)

The polypeptides of the present invention inhibit proliferation of lung cancer cells. Therefore, the present invention provides therapeutic and/or preventive agents for cancer which include as an active ingredient a polypeptide which includes YWITRRFVNQLNTKYRDSLP (SEQ ID NO.:28); or a polynucleotide encoding the same. Alternatively, the present invention relates to methods for treating and/or preventing lung cancer including the step of administering a polypeptide of the present invention. Furthermore, the present invention relates to the use of the polypeptides of the present invention in manufacturing pharmaceutical compositions for treating and/or preventing lung cancer. Furthermore, the present invention also relates to a polypeptide selected from peptides including YWITRRFVNQLNTKYRDSLP (SEQ ID NO.:28) for treating and/or preventing lung cancer.

Alternatively, the inhibitory polypeptides of the present invention can be used to induce apoptosis of cancer cells. Therefore, the present invention provides apoptosis inducing agents for cells, which include as an active ingredient a polypeptide which includes YWITRRFVNQLNTKYRDSLP (SEQ ID NO.:28); or a polynucleotide encoding the same. The apoptosis inducing agents of the present invention may be used for treating cell proliferative diseases such as cancer. Alternatively, the present invention relates to methods for inducing apoptosis of cells which include the step of administering the polypeptides of the present invention. Furthermore, the present invention relates to the use of polypeptides of the present invention in manufacturing pharmaceutical compositions for inducing apoptosis in cells. The inhibitory polypeptides of the present invention induce apoptosis in TTK-expressing cells such as lung cancer. In the meantime, TTK expression has not been observed in most of normal organs. In some normal organs, the expression level of TTK is relatively low as compared with lung cancer tissues. Accordingly, the polypeptides of the present invention may induce apoptosis specifically in lung cancer cells.

When the polypeptides of the present invention are administered, as a prepared pharmaceutical, to human and other mammals such as mouse, rat, guinea pig, rabbit, cat, dog, sheep, pig, cattle, monkey, baboon and chimpanzee for treating lung cancer or inducing apoptosis in cells, isolated compounds can be administered directly, or formulated into an appropriate dosage form using known methods for preparing pharmaceuticals. For example, if necessary, the pharmaceuticals can be orally administered as a sugar-coated tablet, capsule, elixir, and microcapsule, or alternatively parenterally administered in the injection form that is a sterilized solution or suspension with water or any other pharmaceutically acceptable liquid. For example, the compounds can be mixed with pharmacologically acceptable carriers or media, specifically sterilized water, physiological saline, plant oil, emulsifier, suspending agent, surfactant, stabilizer, corrigent, excipient, vehicle, preservative, and binder, in a unit dosage form necessary for producing a generally accepted pharmaceutical. Depending on the amount of active ingredient in these formulations, a suitable dose within the specified range can be determined.

Examples of additives that can be mixed in tablets and capsules are binders such as gelatin, corn starch, tragacanth gum, and gum arabic; media such as crystalline cellulose; swelling agents such as corn starch, gelatin, and alginic acid; lubricants such as magnesium stearate; sweetening agents such as sucrose, lactose or saccharine; and corrigents such as peppermint, wintergreen oil and cherry. When the unit dosage from is capsule, liquid carriers such as oil can be further included in the above-described ingredients. Sterilized mixture for injection can be formulated using media such as distilled water for injection according to the realization of usual pharmaceuticals.

Physiological saline, glucose, and other isotonic solutions containing adjuvants such as D-sorbitol, D-mannose, D-mannitol, and sodium chloride can be used as an aqueous solution for injection. They can be used in combination with a suitable solubilizer, for example, alcohol, specifically ethanol and polyalcohols such as propylene glycol and polyethylene glycol, non-ionic surfactants such as Polysorbate 80TM and HCO-50.

Sesame oil or soybean oil can be used as an oleaginous liquid, and also used in combination with benzyl benzoate or benzyl alcohol as a solubilizer. Furthermore, they can be further formulated with buffers such as phosphate buffer and sodium acetate buffer; analgesics such as procaine hydrochloride; stabilizers such as benzyl alcohol and phenol; and antioxidants. Injections thus prepared can be loaded into appropriate ampoules.

Methods well-known to those skilled in the art can be used for administering pharmaceutical compounds of the present invention to patients, for example, by intraarterial, intravenous, or subcutaneous injection, and similarly, by intranasal, transtracheal, intramuscular, or oral administration. Doses and administration methods are varied depending on the body weight and age of patients as well as administration methods. However, those skilled in the art can routinely select them. DNA encoding a polypeptide of the present invention can be inserted into a vector for the gene therapy, and the vector can be administered for treatment. Although doses and administration methods are varied depending on the body weight, age, and symptoms of patients, those skilled in the art can appropriately select them. For example, a dose of the compound which bind to the polypeptides of the present invention so as to regulate their activity is, when orally administered to a normal adult (body weight 60 kg), about 0.1 mg to about 100 mg/day, preferably about 1.0 mg to about 50 mg/day, more preferably about 1.0 mg to about 20 mg/day, although it is slightly varied depending on symptoms.

When the compound is parenterally administered to a normal adult (body weight 60 kg) in the injection form, it is convenient to intravenously inject a dose of about 0.01 mg to about 30 mg/day, preferably about 0.1 mg to about 20 mg/day, more preferably about 0.1 mg to about 10 mg/day, although it is slightly varied depending on patients, target organs, symptoms, and administration methods. Similarly, the compound can be administered to other animals in an amount converted from the dose for the body weight of 60 kg.

By screening for candidate compounds that (i) bind to TBC1D7; (ii) suppress the biological activity of TBC1D7; (iii) alter the expression level of TBC1D7; (iv) inhibit the binding between TBC1D7 and RAB17, 14-3-3 zeta or TSC1, candidate compounds that have the potential to treat or prevent cancers (e.g., lung cancer and esophageal cancer) can be identified. Potential of these candidate compounds to treat or prevent cancers may be evaluated by second and/or further screening to identify therapeutic agent for cancers. For example, when a compound having the activity of any one of (i) to (iv) above, for example, a compound that binds to the TBC1D7, inhibits the above-described activities of cancer, it may be concluded that such a compound has the TBC1D7-specific therapeutic effect.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

The invention will be further described in the following examples, which do not limit the scope of the invention described in the claims.

### EXAMPLES

Materials and Methods

Cell Lines and Clinical Tissue Samples

The human lung-cancer cell lines used in this study were as follows: lung adenocarcinomas (ADCs) NCI-H1781, NCI-H1373, LC319, A549, and PC14; lung squamous-cell carcinomas (SCCs) SK-MES-1, NCI-H2170, NCI-H520, NCI-H1703, and LU61; a lung large-cell carcinoma (LCC) LX1; and small-cell lung cancers (SCLCs) SBC-3, SBC-5, DMS273, and DMS114. The human esophageal carcinoma cell lines used in this study were as follows: nine SCC cell lines (TE1, TE2, TE3, TE4, TE5, TE6, TE8, TE9, and TE10) and one ADC cell line (TE7) (Nishihira T. et al. J Cancer Res Clin Oncol 1993; 119:441-449). All cells were grown in monolayers in appropriate medium supplemented with 10% fetal calf serum (FCS) and were maintained at 37 degrees C. in atmospheres of humidified air including 5% CO₂. Human small airway epithelial cells (SAEC) were grown in optimized medium (SAGM) purchased from Cambrex Bio Science Inc. Primary lung and esophageal cancers were obtained with informed consent along with adjacent normal lung-tissue samples from patients, as described previously. A total of 270 NSCLCs or 261 NSCLCs (153 ADCs, 89 SCCs, 3 adenosquamous carcinomas, 16 LCCs; 88 female and 173 male patients; median age of 65.0 with a range of 26 to 84 years; 112 pT1, 121 pT2, 28 pT3 tumor size; 203 pNO, 23 pN1, 35 pN2 node status) and adjacent normal lung-tissue samples for immunostaining on tissue microarray were also obtained from patients who had undergone surgery at Hokkaido University and its affiliated hospitals (Sapporo, Japan). This study and the use of all clinical materials were approved by individual institutional ethical committees.

Semi-Quantitative RT-PCR Analysis

Total RNA was extracted from cultured cells and clinical tissues using Trizol reagent (Life Technologies, Inc.) according to the manufacturer's protocol. Extracted RNAs were treated with DNase I (Nippon Gene) and reversely-transcribed using oligo (dT) primer and SuperScript II reverse transcriptase (Invitrogen). Semiquantitative RT-PCR experiments were carried out with the following synthesized TBC1D7-specific primers or with beta-actin (ACTB)-specific primers as an internal control: TBC1D7, 5′-CCCTAGTTTTTGTAGCTGTCGAA-3′ (SEQ ID NO.: 5) or 5′-CCTAGTTTTTGTAGCTGTCGAA-3′ (SEQ ID NO.:15) and 5′-GATCACATGCCAAGAACACAAT-3′ (SEQ ID NO.: 6) ; TSC1, 5′-CTCCACAGCCAGATCAGACA-3′ (SEQ ID NO.:16) and 5′-GCTGCCTGTTCAAGAACTCC-3′ (SEQ ID NO.:17); ACTB, 5′-GAGGTGATAGCATTGCTTTCG-3′ (SEQ ID NO.: 7) and 5′-CAAGTCAGTGTACAGGTAAGC-3′ (SEQ ID NO.: 8). PCR reactions were optimized for the number of cycles to ensure product intensity within the logarithmic phase of amplification.

Northern-Blot Analysis

Human multiple-tissue blots (BD Biosciences Clontech) were hybridized with a ³² P-labeled PCR product of TBC1D7. The cDNA probes of TBC1D7 were prepared by RT-PCR using the primers described above. Pre-hybridization, hybridization, and washing were performed according to the supplier's recommendations. The blots were autoradiographed with intensifying screens at −80 degrees C. for one week.

Anti-TBC1D7 Antibodies

Plasmids expressing full length fragments of TBC1D7 that contained His-tagged epitopes at their NH₂-terminals were prepared using pET28 vector (Novagen, Madison, Wis.). The recombinant peptides were expressed in Escherichia coli, BL21 codon-plus strain (Stratagene, LaJolla, Calif.), and purified using TALON resin (BD Bioscience) according to the supplier's protocol. The protein was inoculated into rabbits; the immune sera were purified on affinity columns according to standard methodology. Affinity-purified anti-TBC1D7 antibodies were used for western blotting as well as immunocytochemical and immunohistochemical studies. It was confirmed that the antibody was specific to TBC1D7 on western blots using lysates from cell lines that had been transfected with TBC1D7 expression vector and those from lung cancer cell lines, either of which expressed TBC1D7 endogenously or not, as well as by immunocytochemical staining of the cell lines.

Western-Blotting

Cells were lysed in lysis buffer; 50 mM Tris-HCl (pH 8.0), 150 mM NaCl, 0.5% NP-40, 0.5% deoxycholate-Na, 0.1% SDS, plus protease inhibitor (Protease Inhibitor Cocktail Set III; Calbiochem). It was used an ECL western-blotting analysis system (GE Healthcare Bio-sciences), as previously described (Takahashi K. et al. Cancer Res 2006; 66:9408-19).

Immunocytochemical Analysis

Cultured cells were washed twice with PBS(−), fixed in 4% paraformaldehyde solution for 30 minutes at 37 degrees C., and then rendered permeable with PBS(−) containing 0.1% Triton X-100 for 3 minutes. Prior to the primary antibody reaction, cells were covered with blocking solution [3% bovine serum albumin in PBS(−)] for 7 or 10 minutes to block nonspecific antibody binding. After the cells were incubated with antibodies to human TBC1D7 (generated to recombinant TBC1D7; please see above), Alexa Fluor 488 goat anti-rabbit secondary antibody (Molecular Probes) was added to reveal endogenous TBC1D7. Nuclei were stained with 4′,6-diamidino-2-phenylindole. The antibody-stained cells were viewed with a laser-confocal microscope (TSC SP2 AOBS: Leica Microsystems).

Immunohistochemistry and Tissue Microarray Analysis

To investigate the presence of TBC1D7 protein in clinical materials, the present inventors stained tissue sections using ENVISION+ Kit/HRP (DakoCytomation, Glostrup, Denmark). For antigen retrieval, slides were immersed in Target Retrieval Solution High pH (DakoCytomation) and boiled at 108 degrees C. for 15 min in an autoclave. 7 or 12 micro g/ml of affinity-purified rabbit polyclonal anti-TBC1D7 antibodies (generated to recombinant TBC1D7; please see above) were added after blocking of endogenous peroxidase and proteins, and each section was incubated with HRP-labeled anti-rabbit IgG as the secondary antibody. Substrate-chromogen was added and the specimens were counterstained with hematoxylin. Tumor-tissue microarrays were constructed as published elsewhere (Chin S F. et al. Molecular Pathology: MP 2003; 56:275-279, Callagy G. et al. Diagnostic Molecular Pathology 2003; 12:27-34, Callagy G. et al. J Pathol 2005; 205:388-396), using formalin-fixed archived NSCLCs obtained by a single institutional group with an identical protocol to collect and fix the tissues after resection (Suzuki C. et al. Cancer Res 2003; 63:7038-41, Takahashi K. et al. Cancer Res 2006; 66:9408-19, Mizukami Y. et al. Cancer Sci 2008; 99:1448-54. Suzuki C. et al. Cancer Res 2003; 63:7038-41, Ishikawa N. et al. Clin Cancer Res 2004; 10:8363-70, Kato T. et al. Cancer Res 2005; 65:5638-46, Furukawa C. et al. Cancer Res 2005; 65:7102-10, Ishikawa N. et al. Cancer Res 2005; 65:9176-84, Suzuki C. et al. Cancer Res 2005; 65:11314-25, Ishikawa N. et al. Cancer Sci 2006; 97:737-45, Takahashi K. et al. Cancer Res 2006; 66:9408-19, Hayama S. et al. Cancer Res 2006; 66:10339-48, Kato T. et al. Clin Cancer Res 2007; 13:434-42, Suzuki C. et al. Mol Cancer Ther 2007; 6:542-51, Yamabuki T. et al. Cancer Res 2007; 67:2517-25, Hayama S. et al. Cancer Res 2007; 67:4113-22, Kato T. et al. Cancer Res 2007; 67:8544-53, Taniwaki M. et al. Clin Cancer Res 2007; 13:6624-31, Ishikawa N. et al. Cancer Res 2007; 67:11601-11, Mano Y. et al. Cancer Sci 2007; 98:1902-13, Suda T. et al. Cancer Sci 2007; 98:1803-8, Kato T. et al. Clin Cancer Res Res 2008; 14:2363-70, Mizukami Y. et al. Cancer Sci 2008; 99:1448-54). Considering the histological heterogeneity of individual lung tumors, tissue areas for sampling were selected based on visual alignment with the corresponding HE-stained sections on slides. Three, four, or five tissue cores (diameter 0.6 mm; height 3-4 mm) taken from donor-tumor blocks were placed into recipient paraffin blocks using a tissue microarrayer (Beecher Instruments, Sun Prairie, Wis.). A core of normal tissue was punched from each case. Five-micro m sections of the resulting microarray block were used for immunohistochemical analysis. Three independent investigators assessed TBC1D7 positivity semiquantitatively without prior knowledge of clinicopathological data. The intensity of TBC1D7 staining was evaluated using the following criteria: strong positive (2+), dark brown staining in more than 50% of tumor cells completely obscuring nucleus and cytoplasm; weak positive (1+), any lesser degree of brown staining appreciable in nucleus and cytoplasm; absent (scored as 0), no appreciable staining in tumor cells. Cases were accepted only as strongly positive if the three reviewers independently defined them as such.

Statistical Analysis

Statistical analyses were performed using the StatView statistical program (SaS). Contingency tables were used to correlate clinicopathological variables (age, gender, histological type, and pathological TNM stage) with the expression levels of TBC1D7 determined by tissue-microarray analysis. Survival curves were calculated from the date of surgery to the time of death related to NSCLC, or to the last follow-up observation. Kaplan-Meier curves were calculated for each relevant variable and for TBC1D7 expression; differences in survival times among patient subgroups were analyzed using the log-rank test. Univariate and multivariate analyses were performed with the Cox proportional-hazard regression model to determine associations between clinicopathological variables and cancer-related mortality. First, the present inventors analyzed associations between death and possible prognostic factors including age, gender, histological type, pT-classification, and pN-classification, taking into consideration one factor at a time. Second, multivariate Cox analysis was applied on backward (stepwise) procedures that always forced TBC1D7 expression into the model, along with any and all variables that satisfied an entry level of a P value less than 0.05. As the model continued to add factors, independent factors did not exceed an exit level of P<0.05.

RNA Interference Assay

(Experimental 1)

Small interfering RNA (siRNA) duplexes (Dharmacon, Inc.) (100 nM) were transfected into a NSCLC cell line, A549 and esophageal cancer cell line, TE9, using 24 of Lipofectamine 2000 (Invitrogen) following the manufacturer's protocol. The transfected cells were cultured for 7 days, and the number of colonies was counted by Giemsa staining, and viability of cells was evaluated by 3-(4,5-dimethylthiazol-2-yl)-2,5-diphenyltetrazolium bromide (MTT) assay (cell counting kit-8 solution; Dojindo Laboratories), at 7 days after transfection. To confirm suppression of TBC1D7 expression, semiquantitative RT-PCR was carried out with synthesized primers specific for TBC1D7 described above. SiRNA duplexes against human TBC1D7, si-TBC1D7-#1: siGenome duplexes 1 [D-021140-01: GAACAGUGCAGAGAAGAUAUU] (SEQ ID NO: 3 for target sequence SEQ ID NO.: 18) and si-TBC1D7-#2: siGenome duplexes 4 [D-021140-4: GAUAAAGUUGUGAGUGGAUUU] (SEQ ID NO.: 4 for target sequence SEQ ID NO.: 19) were purchased from Dharmacon. It was also designed siRNA oligonucleotides against control 1 (EGFP: enhanced green fluorescent protein (GFP) gene, a mutant of Aequorea victoria GFP), 5′-NNGAAGCAGCACGACUUCUUC-3′ (for target sequence SEQ ID NO.: 9) and control 2 (Scramble (SCR): chloroplast Euglena gracilis gene coding for 5S and 16S rRNAs) 5′-NNGCGCGCUUUGUAGGAUUCG (for target sequence SEQ ID NO.: 10).

(Experimental 2)

Small interfering RNA (siRNA) duplexes (100 nM) were transfected into a NSCLC cell line, A549 and LC319, using 24 microL of Lipofectamine 2000 (Invitrogen) following the manufacturer's protocol. The transfected cells were cultured for 7 days, and the number of colonies was counted by Giemsa staining, and viability of cells was evaluated by 3-(4,5-dimethylthiazol-2-yl)-2,5-diphenyltetrazolium bromide (MTT) assay (cell counting kit-8 solution; Dojindo Laboratories), at 7 days after transfection. To confirm suppression of TBC1D7 and TSC1 expression, western blotting using antibodies to TBC1D7 and TSC1 as well as semiquantitative RT-PCR with synthesized primers specific for TBC1D7 and TSC1 were carried out as described above. SiRNA sequences against human TBC1D7 and TSC1 were as follows, si-TBC1D7#3: GAACAGUGCAGAGAAGAUA (SEQ ID NO.:18), si-TBC1D7#4: GAUAAAGUUGUGAGUGGAU (SEQ ID NO.:19), and si-TSC1: CGACACGGCUGAUAACUGA (SEQ ID NO.:20). The present inventors also designed siRNA oligonucleotides against control-1 (LUC: luciferase gene from Photinus pyralis), 5′-CGUACGCGGAAUACUUCGA-3′ (SEQ ID NO.:21) and control-2 (EGFP: enhanced green fluorescent protein (GFP) gene, a mutant of Aequorea victoria GFP), 5′-GAAGCAGCACGACUUCUUC-3′ (SEQ ID NO.:22).

Flow Cytometry

Cells were collected in PBS, and fixed in 70% cold ethanol for 30 minutes. After treatment with 100 micro g/mL RNase (Sigma/Aldrich, St. Louis, Mo.), the cells were stained with 50 micro g/mL propidium iodide (Sigma/Aldrich, St. Louis, Mo.) in PBS. Flow cytometry was done on a Becton Dickinson FACScan and analyzed by ModFit software (Verity Software House, Inc., Topsham, Me.). The cells selected from at least 10,000 ungated cells were analyzed for DNA content.

Establishment of TBC1D7-expressing COS-7 transfectants and their growth in vitro

(Experimental 1)

TBC1D7-expressing stable transfectants were established according to a standard protocol. The entire coding region of TBC1D7 was amplified by RT-PCR. The product was digested with EcoRI and XhoI, and cloned into appropriate sites of a pCAGGSn3FC vector that contained 3×flag-epitope sequences at the C-terminal of the TBC1D7 protein. Using FuGENE 6 Transfection Reagent (Roche Diagnostics) according to the manufacturer's instructions, COS-7 cells were transfected with plasmids expressing either TBC1D7 (pCAGGS-TBC1D7-flag) or mock plasmids (pCAGGSn3FC). Transfected cells were cultured in medium containing 10% FCS and geneticin (0.8 mg/ml) for 14 days; then 50 individual colonies were trypsinized and screened for stable transfectants by a limiting-dilution assay. Expression of TBC1D7 was determined in each clone by western blotting and immunocytochemistry. Cell viability of two stable clones (COS-7-TBC1D7-#1 and -#2) and two control clones (COS-7-mock-#1 and -#2) was quantified with MTT assay in 24, 72, 120, and 168 hours.

(Experimental 2)

TBC1D7-expressing stable transfectants were established according to a standard protocol. The entire coding region of TBC1D7 was amplified by RT-PCR. The product was digested with EcoRI and XhoI, and cloned into appropriate sites of a pCAGGSn3FC vector that contained 3×flag-epitope sequences at the C-terminal of the TBC1D7 protein. Using FuGENE 6 Transfection Reagent (Roche Diagnostics) according to the manufacturer's instructions, COS-7 cells were transfected with plasmids expressing either TBC1D7 (pCAGGS-TBC1D7-flag) or mock plasmids (pCAGGSn3FC). Transfected cells were cultured in medium containing 10% FCS and geneticin (0.6 mg/ml) for 14 days; then 50 individual colonies were trypsinized and screened for stable transfectants by a limiting-dilution assay. Expression of TBC1D7 was determined in each clone by western blotting and immunocytochemistry. Cell viability of two stable clones (COS-7-TBC1D7-#A and -#B) and two control clones (COS-7-mock-#A and -#B) was quantified with MTT assay in 24, 72, 120, and 168 hours.

Matrigel Invasion Assay

COS-7-TBC1D7-#1 or COS-7-mock-#1 was grown to near confluence in DMEM containing 10% FCS. The cells were harvested by trypsinization, washed in DMEM without addition of serum or proteinase inhibitor, and suspended in DMEM at concentration of 2×10⁵ cells/mL. Before preparing the cell suspension, the dried layer of Matrigel matrix (Becton Dickinson Labware) was rehydrated with DMEM for 2 hours at room temperature. DMEM (0.75 mL) containing 10% FCS was added to each lower chamber in 24-well Matrigel invasion chambers, and 0.5 mL (1×10⁵ cells) of the cell suspension was added to each insert of the upper chamber. The plates of inserts were incubated for 22 hours at 37 degrees C. and the chambers were processed; cells invading through the Matrigel were fixed and stained by Giemsa as directed by the supplier (Becton Dickinson Labware).

Mice Model

The animal experiments were conducted according to the institutional and national guidelines for the care and use of laboratory animals, and approved by the institutional animal use committee. To examine in vivo tumor formation by TBC1D7 overexpression, above established COS-7 cells stably expressing TBC1D7 or those transfected with mock plasmids (1×107) were injected subcutaneously into the posterior mid-dorsum of 8 BALB/cAJcl-nu/nu mice (male, 7 weeks old). Subsequently, the mice were euthanized at 60 days after cell transplantation, and the tumors were dissected.

Identification of TBC1D7-Associating Protein

Cell extracts from COS-7-TBC1D7-#A or COS-7-Mock-#A were precleared by incubation at 4 degrees C. for 1 hour with 100 microL of protein G-agarose beads in a final volume of 1 mL of immunoprecipitation buffer (0.5% NP-40, 50 mM Tris-HCl, 150 mM NaCl) in the presence of proteinase inhibitor. After centrifugation at 1000 rpm for 1 min at 4 degrees C., the supernatant was incubated at 4 degrees C. with anti-Flag M2 agarose beads for 2 hours. The beads were then collected by centrifugation at 5000 rpm for 1 min and washed six times with 1 mL of each immunoprecipitation buffer. The washed beads were resuspended in 20 microL of Laemmli sample buffer and boiled for 5 min, and the proteins were separated in 5-10% SDS polyacrylamide gel electrophoresis (PAGE) gels (BIO RAD). After electrophoresis, the gels were stained with silver. Protein band specifically found in COS-7-TBC1D7-#A extracts immunoprecipitated with anti-Flag M2 agarose beads was excised and served for matrix-assisted laser desorption/ionization-time of flight mass spectrometry (MALDI-TOF-MS) analysis (AXIMA-CFR plus, SHIMADZU BIOTECH).

Dominant-Negative Peptide Assay

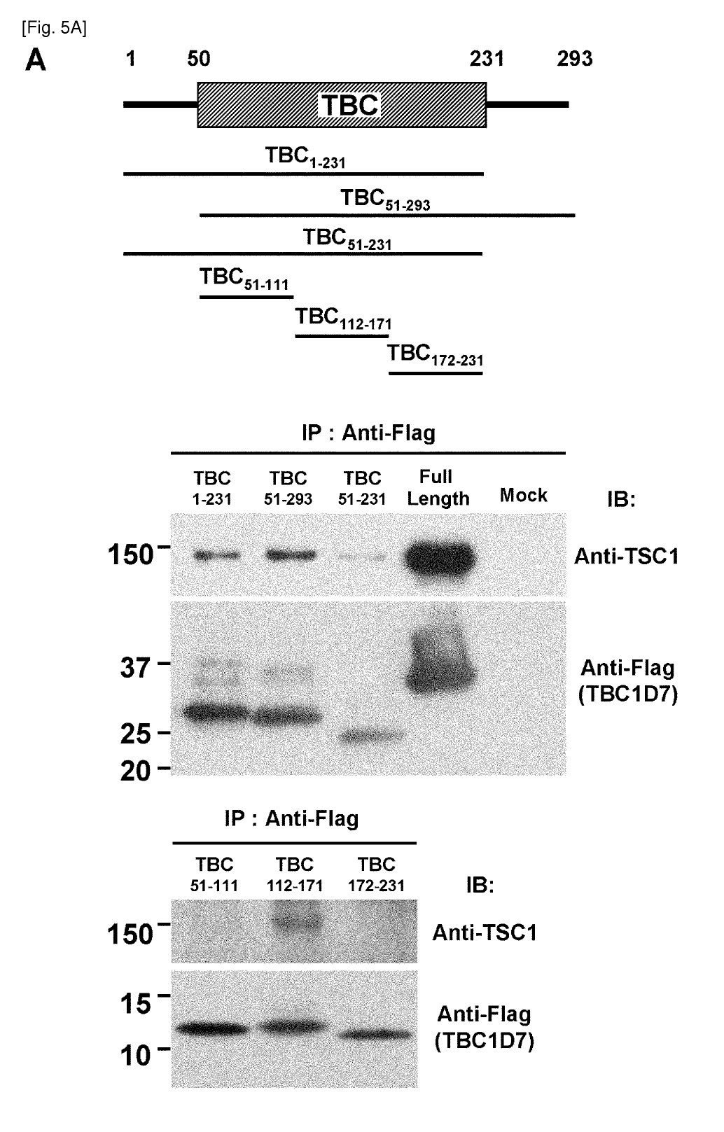

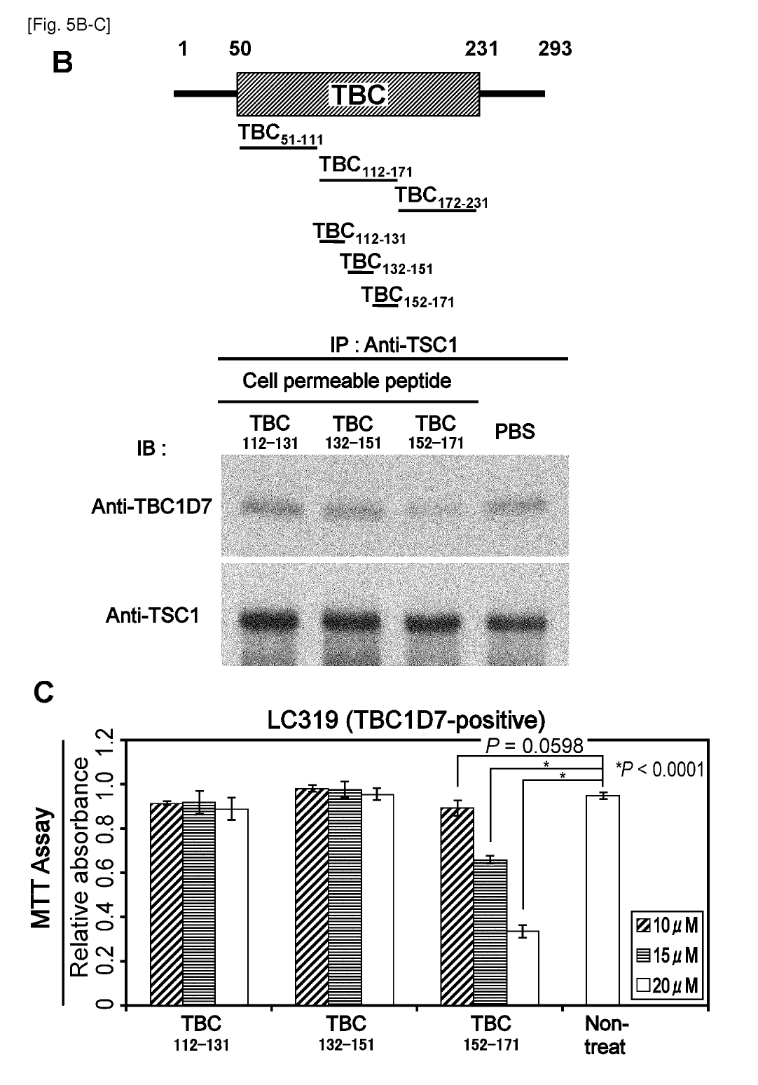

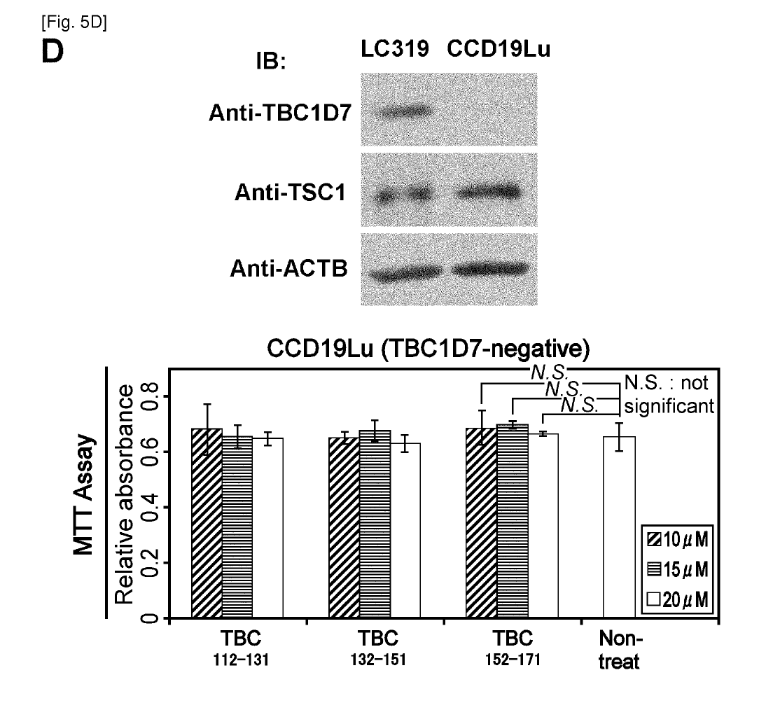

Twenty-amino-acid sequence derived from minimized TSC1-binding domain in TBC1D7 (codons 112-171; see FIG. 5B) was covalently linked at its N-terminus to a membrane transducing 11 poly-arginine sequence (11R) as described elsewhere (Hayama S, Daigo Y, Kato T, et al. Cancer Res 2006; 66:10339-48, Hayama S, Daigo Y, Yamabuki T, et al. Cancer Res 2007; 67:4113-22). Three dominant-negative peptides were synthesized covering the codons 112-171 region: 11R-TBC112-131, RRRRRRRRRRR-GGG-YQLESGKLPRSPSFPLEPDD (SEQ ID NO.:23); 11R-TBC132-151, RRRRRRRRRRR-GGG-EVFLAIAKAMEEMVEDSVDC (SEQ ID NO.:24); 11R-TBC152-171 RRRRRRRRRRR-GGG-YWITRRFVNQLNTKYRDSLP (SEQ ID NO.:25). Peptides were purified by preparative reverse-phase high-performance liquid chromatography to make >95% purity. Lung cancer LC319 cells that expressed both TBC1D7 and TSC1 as well as normal human lung fibroblast-derived CCD19Lu that did not express TBC1D7 were incubated with the 11R peptides at the concentration of 10, 15 or 20 microM for 3 days. The viability of cells was evaluated by MTT assay at 3 days after the treatment.

Results

TBC1D7 expression in lung cancers and normal tissues

Using a cDNA microarray representing 27,648 genes, TBC1D7 was identified to be highly transactivated in the majority of lung cancers, while it was detected only in testis and fetal liver. The present inventors subsequently confirmed its transactivation by semiquantitative RT-PCR experiments in 14 of 15 lung cancer tissues (5 of 5 ADCs; 5 of 5 SCCs; 4 of 5 SCLSs (FIG. 1A). High level of TBC1D7 expression was also observed in 13 of the 15 lung-cancer cell lines examined, while the transcript was hardly detectable in SAEC cells derived from normal airway epithelium (FIG. 1B). It was subsequently confirmed by Western blotting analysis using anti-TBC1D7 antibody overexpression of 30-kDa TBC1D7 protein in lung cancer cell lines (FIG. 1C). To examine the subcellular localization of endogenous TBC1D7 in lung cancer LC319 cells, it was performed immunofluorescence analysis using anti-TBC1D7 antibody and LC319 cells. TBC1D7 was localized in the nucleus and the cytoplasm (FIG. 1D). Northern-blot analysis using TBC1D7 cDNA as a probe identified a 1.35-kb transcript exclusively and abundantly in testis among the 16 normal human adult tissues (FIG. 1E). Furthermore, it was compared by immunohistochemistry using anti-TBC1D7 polyclonal antibodies TBC1D7 protein expressions in 5 normal tissues (heart, lung, liver, kidney, and testis) with those in lung cancers. TBC1D7 expressed abundantly in testis (in nucleus and cytoplasm of spermatocytes) and lung cancers, but its expression was hardly detectable in the remaining four normal tissues (FIG. 1F).

Association of TBC1D7 Expression with Poor Prognosis for NSCLC Patients

(Experimental 1)

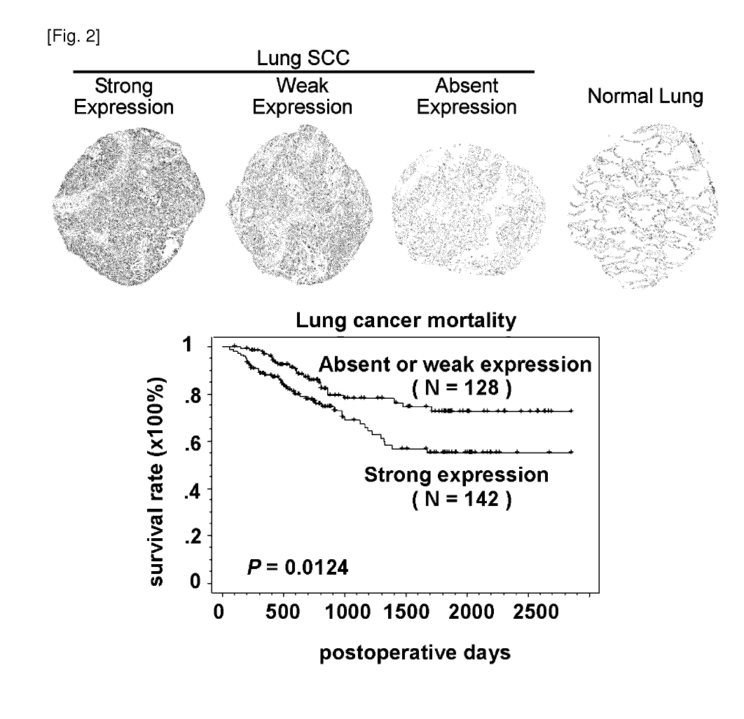

Using tissue microarrays prepared from paraffin-embedded NSCLCs, it was performed immunohistochemical analysis with affinity-purified anti-TBC1D7 polyclonal antibodies. The present inventors classified patterns of TBC1D7 expression as negative or positive. Of the 270 NSCLC cases examined, 142 (52.6%) revealed positive and 128 (47.4%) revealed negative TBC1D7 staining in nucleus and cytoplasm in NSCLC cells, but no staining was observed in any of their adjacent normal lung cells or stromal cells (FIG. 2, top panels). It was then examined the association of TBC1D7 expression with various clinicopathological parameters of NSCLC patients who had undergone curative surgery, and found its significant correlation with gender (higher in male, P=0.0051), histopathologic type (higher in non-ADC, P<0.0001), tumor size (higher in T2+T3+T4, P<0.0001), and node status (higher in N1+N2; Table 1A). The Kaplan-Meier analysis indicated a significant association between TBC1D7-positivity in NSCLCs and worse tumor-specific survival (P=0.0124 by log-rank test; FIG. 2, bottom panels). We also applied univariate analysis to evaluate associations between patient prognosis and several factors including age, gender, histological type (ADC versus non-ADC), pT stage (tumor size; T1 versus T2+T3+T4), pN stage (node status; NO versus N1+N2), and TBC1D7 status (absent or weak expression versus strong expression). All those parameters were significantly associated with poor prognosis (Table 1B). In multivariate analysis, TBC1D7 status did not reach the statistically significant level as independent prognostic factor for surgically treated lung cancer patients enrolled in this study (P=0.7586), suggesting the relevance of TBC1D7 expression to these clinicopathological factors in lung cancer (Table 1B).

TABLE 1A
Association between TBC1D7-positivity in NSCLC tissues
and patients' characteristics (n = 270)

| | | TBC1D7 | TBC1D7 | | P-value |
|---|---|---|---|---|---|
| | Total | positive | negative | | positive vs |
| | n = 270 | n = 142 | n = 128 | χ2 | negative |
| Age (years) | | | | | |
| <65 | 134 | 64 | 70 | 2.491 | 0.1145 |
| >=65 | 136 | 78 | 58 | | |
| Gender | | | | | |
| Female | 91 | 37 | 54 | 7.84 | 0.0051* |
| Male | 179 | 105 | 74 | | |
| Histological | | | | | |
| type | | | | | |
| ADC | 158 | 58 | 100 | 38.542 | <0.0001* |
| non-ADC | 112 | 84 | 28 | | |
| pT factor | | | | | |
| T1 | 112 | 40 | 72 | 21.868 | <0.0001* |
| T2 + T3+ T4 | 158 | 102 | 56 | | |
| pN factor | | | | | |
| N0 | 207 | 100 | 107 | 6.528 | 0.0106* |
| N1 + N2 | 63 | 42 | 21 | | |

ADC, adenocarcinoma
non-ADC, squamous-cell carcinoma plus large-cell carcinoma and adenosquamous-cell carcinoma
P < 0.05 (chi-square test)

TABLE 1B
Cox's proportional hazards model analysis of prognostic factors
in patients with NSCLCs

| | Hazards | | Unfavorable/ | |
|---|---|---|---|---|
| Variables | ratio | 95% CI | Favorable | P-value |
| Univariate | | | | |
| analysis | | | | |
| TBC1D7 | 1.867 | 1.135-3.071 | Positive/Negative | 0.0139* |
| Age (years) | 2.227 | 1.357-3.653 | > = 65/65> | 0.0015* |
| Gender | 2.288 | 1.287-4.068 | Male/Female | 0.0048* |
| Histological type | 2.638 | 1.611-4.319 | non-ADC/ADC | 0.0001* |
| pT factor | 3.752 | 2.046-6.880 | T2 + T3 + T4/T1 | <0.0001* |
| pN factor | 4.304 | 2.658-6.971 | N1 + N2/N0 | <0.0001* |
| Multivariate | | | | |
| analysis | | | | |
| TBC1D7 | 0.919 | 0.535-1.577 | Positive/Negative | 0.7586 |
| Age (years) | 1.846 | 1.114-3.059 | > = 65/65> | 0.0173* |
| Gender | 1.400 | 0.736-2.664 | Male/Female | 0.3049 |
| Histological type | 1.712 | 0.948-3.091 | non-ADC/ADC | 0.0745 |
| pT factor | 2.160 | 1.138-4.100 | T2 + T3 + T4/T1 | 0.0185* |
| pN factor | 3.62 | 2.215-5.917 | N1+ N2/N0 | <0.0001* |

ADC, adenocarcinoma
non-ADC, squamous-cell carcinoma plus large-cell carcinoma and adenosquamous-cell carcinoma
P < 0.05

Tumor Cell Growth Suppression by siRNA for TBC1D7

It was used several siRNA expression oligonucleotides specific to TBC1D7 sequences and transfected them into LC319 and A549 cell lines that endogenously expressed high levels of TBC1D7. A knockdown effect was confirmed by RT-PCR when the present inventors used si-TBC1D7-#1 and si-TBC1D7-#2 constructs (FIG. 3A, top panels). MTT assays and colony-formation assays revealed a drastic reduction in the number of cells transfected with si-TBC1D7-#1, #2 (FIG. 3A, middle and bottom panels). Flowcytometric analysis revealed that 48 to 96 hours after the transfection of si-TBC1D7 to the lung cancer LC319 cells, the number of cells in S phase was continuously decreased, while the proportion of the cells in G2/M phase were increased during 48 to 96 hours after the transfection (FIG. 3B).

Activation of Cellular Growth and Invasion Activity by TBC1D7

As the immunohistochemical analysis on tissue microarray indicated that lung cancer patients with TBC1D7 strong-positive tumors showed a shorter cancer-specific survival period than those with TBC1D7-weak-positive and/or negative tumors, it was examined a possible role of TBC1D7 in cellular growth and invasion. The present inventors transfected plasmids designed to express TBC1D7 (pCAGGS-TBC1D7-flag) into COS-7 cells and established two independent COS-7 cell lines over-expressing exogenous TBC1D7 (COS-7-TBC1D7-#1 and -#2). It was compared their growth with control cells transfected with mock vector (COS-7-mock-#1 and -#2). Growth of the two COS-7-TBC1D7 cells was promoted at a significant degree in accordance with the expression level of TBC1D7 as detected by western blot analysis (FIG. 3C). To further explore the potential oncogenic effect of activation of TBC1D7 on cellular invasion, it was compared their invasive activity using matrigel invasion assays. As shown in FIG. 3D, invasive activity of COS-7-TBC1D7-#1 through Matrigel significantly was enhanced compared with COS-7-mock-#1.

To investigate a potential role of TBC1D7 in vivo tumorigenesis, there were subcutaneously transplanted either COS-7-TBC1D7-#1 cells or COS-7-Mock-#1 cells into BALB/cAJcl-nu/nu mice. During 60 days observation, all 4 mice that were individually transplanted with COS-7-TBC1D7-#1 cells had tumors containing viable cells, while no visible tumor was formed in 4 independent mice transplanted with COS-7-Mock-#1 cells (FIGS. 3E and 3F). These findings imply an in vivo and in vitro oncogenic effect of TBC1D7.

Identification of Molecules Interacting with TBC1D7

(Experimental 1)

To elucidate the biological mechanism of TBC1D7 in lung and esophageal carcinogenesis, the present inventors attempted to identify proteins that would interact with TBC1D7. Because TBC1D7 has consensus mode-1 14-3-3 binding motif, cell extracts from COS-7 cells transiently transfected with flag-TBC1D7-expression vector or mock vector (negative control) were immunoprecipitated with flag M2 agarose, following immunoblotting with anti-14-3-3 zeta antibody (Cell signaling technology, USA). It was subsequently confirmed the cognate interaction of exogenous TBC1D7 with endogenous 14-3-3 zeta (FIG. 4A). On the other hand, TBC1D7 has been recently reported to act on RAB 17 as a cognate GTPase-activating proteins (GAPs) in primary cilia formation, thus it was constructed RAB 17 expression vector (pcDNA3.1 Myc-His RAB17) and confirmed the interaction between TBC1D7 and RAB17 (FIG. 4B).

(Experimental 2)

Interaction of TBC1D7 with TSC1.

To elucidate the biological mechanism of TBC1D7 in lung carcinogenesis, it was attempted to identify proteins that would interact with TBC1D7. Cell extracts from COS-7-TBC1D7-#A used to examine the in vitro growth and in vivo tumorigenic effect of TBC1D7, or COS-7-Mock-#A (negative control) were immunoprecipitated with anti-Flag M2 agarose beads. Following separation by SDS-PAGE, protein complexes were silver-stained. A protein band, which was seen in immunoprecipitates by anti-Flag M2 agarose in COS-7-TBC1D7-#A, but not in negative control cells, was excised, trypsin-digested, and subjected to mass spectrometry analysis. Peptides from the extracted band of 130 kDa matched to parts of TSC1 that were conserved between human and monkey. It was next examined TSC1 expression in human lung-cancer cell lines by semi-quantitative RT-PCR experiments and western-blotting, and found co-expression of TBC1D7 and TSC1 in most of lung-cancer cells examined (FIG. 4C), suggesting the possibility of a complex formation of these two proteins in lung cancer cells. We subsequently confirmed the interaction between endogenous TBC1D7 and endogenous TSC1 in the lung cancer LC319cells by immunoprecipitation using rabbit polyclonal antibodies to TBC1D7 and TSC1 (FIG. 4D).

To further assess whether expression of TSC1 could affect TBC1D7 function in lung cancer cells, it was examined the levels of TBC1D7 after suppression or overexpression of TSC1 in LC319 cells. Treatment of LC319 cells with siRNA oligonucleotides against TSC1 (si-TSC1) suppressed expression of the endogenous TSC1 in comparison to the control siRNA (si-EGFP). Interestingly, the TBC1D7 protein level was decreased in cells treated with si-TSC1, while the transcription level of TBC1D7 was unchanged (FIG. 4E, left panels). On the other hand, overexpression of TSC1 resulted in the increase of TBC1D7 protein, while the expression level of TBC1D7 transcript was unchanged (FIG. 4E, right panels). The decrease of TBC1D7 protein in the cells treated with si-TSC1 was compensated by induction of TSC1-expressing plasmid (FIG. 4F), implying a possibility of stabilization of TBC1D7 protein through its interaction with TSC1 and its contribution to the enhancement of cell growth.

Growth inhibition of lung cancer cells by dominant-negative peptides of TBC1D7

To further investigate the biological importance of the interaction of these two proteins, either of three partial constructs of TBC1D7 were transfected with Flag sequence at its N-terminus (TBC1-231, TBC51-293, and TBC51-231; FIG. 5A, left panel) into COS-7 cells. Immunoprecipitation with monoclonal anti-Flag antibody indicated that all constructs were able to interact with endogenous TSC1 (FIG. 5A, right top panels). To further define the minimal and high-affinity TSC1-binding domain in TBC51-231, either of three additional constructs of TBC1D7 (TBC51-111, TBC112-171, and TBC172-231; FIG. 5A, left panel) was transfected into COS-7 cells and it was found that TBC112-171 was able to interact with TSC1, but TBC51-111 and TBC172-231 were not (FIG. 5A, right bottom panel). These experiments suggested that the 60-amino-acid polypeptide (codons 112-171) in TBC1D7 should play an important role in the interaction with TSC1.

To develop the bioactive cell-permeable peptides that can inhibit the functional association of TBC1D7 with TSC1, it was synthesized three different kinds of 20-amino-acid polypeptides covering the TSC1-binding domain in TBC112-171 with a membrane-permeable 11 residues of arginine (11R) at its N-terminus (11R-TBC1D7112-131, 11R-TBC1D7132-151, and 11R-TBC1D7152-171). To test the effect of these polyarginine-linked peptides on lung cancer cell growth/survival, LC319 was treated with each of the three peptides. Addition of the 11R-TBC1D7152-171 into the culture media inhibited the complex formation between TBC1D7 and TSC1 (FIG. 5B), and resulted in significant decreases in cell viability, as measured by MTT assay (FIG. 5C; P<0.0001 for 15 micro M and <0.0001 for 20 micro M peptide treatment by unpaired t-test). On the other hand, no effect on cell growth was observed when the cells were treated with the remaining two peptides (11R-TBC1D7112-131 and 11R-TBC1D7132-151). 11R-TBC1D7152-171 revealed no effect on cell viability of normal human lung fibroblast derived CCD19Lu cells in which TBC1D7 expression was hardly detectable (FIG. 5D). These data suggested that 11R-TBC1D7152-171 peptides could inhibit a functional complex formation of TBC1D7 and TSC1 and have no off-target toxic effect on normal human cells that do not express TBC1D7 protein.

Discussion

In spite of the development of new molecular-targeting anti-cancer drugs, the proportion of patients having survival benefit is still very limited and some of them could suffer serious adverse effect. Therefore, this invention has established an effective system to identify therapeutic targets for developing small-molecule compounds that have more efficient anti-cancer effect with minimum adverse reaction than current therapies (Kikuchi T. et al. Oncogene 2003; 22:2192-205, Kakiuchi S. et al. Mol Cancer Res 2003; 1:485-99, Kakiuchi S. et al. Hum Mol Genet 2004; 13:3029-43, Kikuchi T. et al. Int J Oncol 2006; 28:799-805, Taniwaki M. et al. Int J Oncol 2006; 29:567-75, Yamabuki T. et al. Int J Oncol 2006; 28:1375-84). The strategy is as follows; 1) To identify up-regulated genes in lung and esophageal cancers by genome-wide screening using the cDNA microarray system, 2) To verify the candidate genes for no or low level of expression in normal tissues by cDNA microarray and northern-blot analyses, 3) To confirm their overexpression in hundreds of archived lung cancer samples by tissue microarray and examine their correlation with clinicopathological factors, 4) To verify whether the target genes are essential for cell growth or the survival of cancer cells by RNAi assay, and 5) To screen the epitopes that enhance cytotoxic T lymphocyte (CTL) from the cancer-specific oncoproteins. By this systematic approach, it was found that TBC1D7 is an oncoprotein that is over-expressed in the great majority of clinical lung and esophageal cancer samples and is essential for carcinogenesis.

The transfection of specific siRNA for TBC1D7 into NSCLC cells reduced its expression and resulted in growth suppression. Concordantly, induction of TBC1D7 in mammalian COS-7 cells promoted the in vitro cell growth and in vivo tumor formation in mice. Moreover, clinicopathological evidence through our tissue-microarray experiments demonstrated that NSCLC patients with tumors strongly expressing TBC1D7 showed shorter cancer-specific survival periods than those with negative or weak TBC1D7 expression. The results obtained by in vitro and in vivo assays demonstrate that overexpressed TBC1D7 is an important molecule in pulmonary tumorigenesis. This is, to our best knowledge, the first study to show the oncogenic function and the prognostic value of TBC1D7.

Proteins containing a TBC domain have been shown to act as GTPase-activating proteins (GAPs) and function through the interaction with Rab-like small G proteins. Many of the Rab proteins are associated with fundamental biological processes such as vesicle fusion, receptor recycling, membrane transport and cytokinesis (Zerial M and McBride H. Nat Rev Mol Cell Biol. 2001; 2:107-17). Each Rab member cycles between the GDP-bound inactive state and GTP-bound active state, and the GTP-bound activated form mediates membrane transport through specific interaction with an effecter molecule(s), thus controlling their function (Zerial M and McBride H. Nat Rev Mol Cell Biol. 2001; 2:107-17, Pfeffer S R. Trends Cell Biol. 2001; 11:487-91, Stenmark H and Olkkonen V M. Genome Biol. 2001; 2 REVIEWS3007). Two key families of enzymes, guanine nucleotide exchange factors (GEFs) and GTPase-activating proteins (GAPs), are generally believed to control the GDP/GTP-cycling of Rabs (Zerial M and McBride H. Nat Rev Mol Cell Biol. 2001; 2:107-17, Segev N. Sci STKE. 2001; 100:RE11). Recently, TBC1D7 has been reported to act on Rabl7 by biochemical GAP assays in primary cilia formation (Yoshimura S. et al. J Cell Biol. 2007; 178:363-9). Rabl7 has been previously reported to be induced during cell polarization and to be involved in the function of apical sorting endosomes in polarized epithelial cells (Lutcke A. et al. J Cell Biol. 1993; 121:553-64, Zacchi P. et al. J Cell Biol. 1998; 140:1039-53), but function of RAB 17 in cancer cells has not been described. Using immunoprecipitation assay, it was confirmed the interaction between TBC1D7 and RAB17. GAPs enhance the inherently slow GTPase activity of G proteins, causing their inactivation and thus modulating the cellular pathways controlled by the respective G proteins (Bernards A. Biochim Biophys Acta 2003; 1603:47-82, Chavrier P, Goud B. Curr Opin Cell Biol 1999; 11:466-75). Some proteins containing a TBC domain have been reported their involvement in carcinogenesis (Pei L, Peng Y, Yang Y, et al. Cancer Res 2002; 62:5420-24). For example, the TRE17 oncogene is expressed in Ewing sarcoma and is involved in actin remodeling as a component of an effecter pathway for Rho GTPases Cdc42 and Rac1 (Masuda-Robens J M, Kutney S N, Qi H, et al. Mol Cell Biol 2003; 23:2151-61).

On the other hand, TBC1D7 has a consensus mode-1 14-3-3 binding motif (RSxpSxP) and it was demonstrated the interaction between TBC1D7 and 14-3-3 zeta using immunoprecipitation assay. 14-3-3 proteins are a family of highly conserved cellular proteins that play key roles in the regulation of central physiological pathways. More than 200 14-3-3 target proteins have been identified, including proteins involved in mitogenic and cell survival signaling, cell cycle control and apoptotic cell death. Importantly, the involvement of 14-3-3 proteins in the regulation of various oncogenes and tumor suppressor genes points to a potential role in human cancer (Tzivion G. et al. Semin Cancer Biol. 2006; 16:203-13). The optimal binding motifs correspond to mode-1 (RSXpSXP) and mode-2 (RXF/YXpSXP, where pS denotes phosphoserine or phosphothreonine) sequences that are recognized by all 14-3-3 isoforms (Gardino A K et al. Semin Cancer Biol. 2006; 16:173-82). No phosphorylation of TBC1D7 was detected in our phosphatase assay (data not shown), suggesting the possibility that this interaction is indirect. Future study of 14-3-3 binding motif in TBC1D7 may help the understanding of the association of these proteins and TBC1D7 oncogenic function(s).

The data herein revealed that TSC1 could interact with and stabilize TBC1D7. The inhibition of the interaction of these molecules with dominant negative cell permeable peptide of TBC1D7 resulted in suppression of cancer cell growth, indicating that this interaction has a crucial role in the growth of cancer cells. Importantly, this permeable peptide has no toxic effect on normal human cells that do not express TBC1D7. Although the detailed function of the TBC1D7-TSC1 complex in cancer cells remains to be clarified, specific inhibition of TBC1D7-TSC1 complex as well as TBC1D7 function is likely to be an effective approach to treat lung cancer.

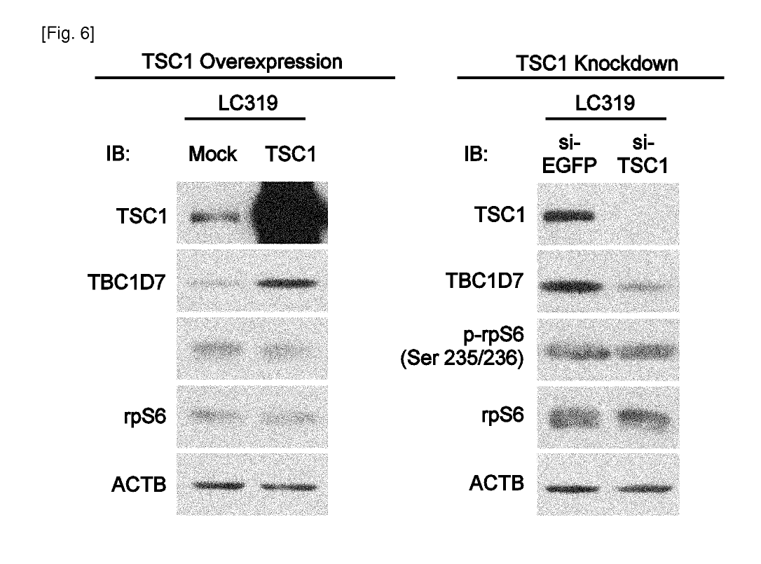

TSC1 encodes a 130-kDa protein and loss of TSC1 in tuberous sclerosis complex (TSC) is responsible for benign tumor syndrome such as hamartoma with a low risk of malignancy (Consortium TEC1T. Cell 1993; 75:1305-15, Crino P B, Nathanson K L, Henske E P. N Engl J Med 2006; 355:1345-56, Huang J, Dibble C C, Matsuzaki M, et al. Mol Cell Biol 2008; 28:4104-15). The TSC1-TSC2 complex plays a central role in signal-integrating nodes within the cell (Huang J, Dibble C C, Matsuzaki M, et al. Mol Cell Biol 2008; 28:4104-15). Recent studies intriguingly suggest that high levels of TSC2 expression were correlated with increased tumor invasiveness and poor prognosis for breast cancer patients (Liu H, Radisky D C, Nelson C M, et al. Proc Natl Acad Sci USA 2006; 103:4134-9). Although it is now clear that the TSC1-TSC2 complex is a critical upstream inhibitor of mTORC1, a role for this complex in the regulation of other downstream targets remains unclear (Huang J, Dibble C C, Matsuzaki M, et al. Mol Cell Biol 2008; 28:4104-15). In fact, loss of the TSC1-TSC2 complex leads to a general reduction in AKT phosphorylation (Huang J, Dibble C C, Matsuzaki M, et al. Mol Cell Biol 2008; 28:4104-15), which may indicate that TSC1 expression plays an important role in cellular survival. To further assess whether expression of TSC1 could affect mTORC1 pathway in lung cancer cells, it was examined the levels of p-rpS6 (Ser235/236), which is downstream target of mTORC1 after suppression or overexpression of TSC1 in LC319 cells. Treatment of LC319 cells with siRNA against TSC1 suppressed expression of the endogenous TSC1 and reduced the levels of TBC1D7 protein, while the levels of p-rpS6 (Ser235/236) protein was not changed (FIG. 6, left panels). On the other hand, overexpression of TSC1 resulted in the increase of TBC1D7 protein, while the levels of p-rpS6 (Ser235/236) protein was not affected (FIG. 6, right panels). These results may suggest that TSC1-TBC1D7 complex function is independent on mTORC1 pathway in lung cancer cells.

In summary, human TBC1D7 has an important functional role in growth/survival and malignant nature of lung and esophageal cancers. Our data provide means for designing new small molecule compounds to specifically target the enzymatic activity of TBC1D7. TBC1D7 overexpression in resected tumor specimens is a useful index as a prognostic biomarker for application of adjuvant therapy to the patients who are likely to have poor prognosis.

### INDUSTRIAL APPLICABILITY

The gene-expression analysis of cancers described herein, using the combination of laser-capture dissection and genome-wide cDNA microarray, has identified specific genes as targets for cancer prevention and therapy. Based on the expression of a subset of these differentially expressed genes, the present invention provides molecular diagnostic markers for identifying and detecting cancers as well as assessing the prognosis.

The methods described herein are also useful for the identification of additional molecular targets for prevention, diagnosis, and treatment of cancers. The data provided herein add to a comprehensive understanding of cancers, facilitate development of novel diagnostic strategies, and provide clues for identification of molecular targets for therapeutic drugs and preventative agents. Such information contributes to a more profound understanding of tumorigenesis, and provides indicators for developing novel strategies for diagnosis, treatment, and ultimately prevention of cancers.

All patents, patent applications, and publications cited herein are incorporated by reference in their entirety.

Furthermore, while the invention has been described in detail and with reference to specific embodiments thereof, it is to be understood that the foregoing description is exemplary and explanatory in nature and is intended to illustrate the invention and its preferred embodiments. Through routine experimentation, one skilled in the art will readily recognize that various changes and modifications can be made therein without departing from the spirit and scope of the invention. Thus, the invention is intended to be defined not by the above description, but by the following claims and their equivalents.

1. A method of detecting or diagnosing cancer in a subject, comprising determining an expression level of TBC1D7 in a patient-derived biological sample, wherein an increase of said level compared to a normal control level of said gene indicates that said subject suffers from or is at risk of developing cancer, wherein the expression level is determined by any one method selected from the group consisting of:
(a) detecting a mRNA of TBC1D7,
(b) detecting a protein encoded by TBC1D7, and
(c) detecting biological activity of the protein encoded by TBC1D7.

2. The method of claim 1, wherein said increase is at least 10% greater than said normal control level.

3. The method of claim 1, wherein the patient-derived biological sample is a biopsy.

4. The method of claim 1, wherein the cancer is selected from the group consisting of lung cancer and esophageal cancer.

5. A kit for detecting or diagnosing cancer, which comprises a detection reagent that binds to a transcription or translation product of TBC 1 D7.

6. A method for assessing prognosis of a patient with lung cancer and/or esophageal cancer, which method comprises the steps of:
(a) detecting expression level of TBC1D7 in a biological sample;
(b) comparing the detected expression level to a control level; and
(c) determining prognosis of the patient based on the comparison of (b).

7. The method of claim 6, wherein the control level is a good prognosis control level and an increase of the expression level compared to the control level is determined as poor prognosis.

8. The method of claim 7, wherein the increase is at least 10% greater than said control level.

9. The method of claim 6, wherein said expression level is determined by any one method selected from the group consisting of:
(a) detecting a mRNA of TBC1D7;
(b) detecting a protein encoded by TBC1D7; and
(c) detecting biological activity of the protein encoded by TBC1D7.

10. A method of screening for a candidate compound for treating or preventing cancer or inhibiting cancer cell growth, said method comprising the steps of:
a) contacting a test compound with a polypeptide encoded by TBC1D7;
b) detecting binding activity between the polypeptide and the test compound or detecting biological activity of the polypeptide of step (a); and
c) selecting a compound that binds to the polypeptide or selecting a compound that suppresses biological activity of the polypeptide in comparison with biological activity detected in absence of the test compound.

11. A method of screening for a candidate compound for treating or preventing cancer or inhibiting cancer cell growth, said method comprising the steps of
a) contacting a test compound with a cell expressing TBC1D7; and
b) selecting a compound that reduces expression level of TBC1D7.

12. (canceled)

13. The method of claim 10, wherein the biological activity is cell proliferative activity or invasion activity.

14. A method of screening for a candidate compound for treating or preventing cancer or inhibiting cancer cell growth, said method comprising the steps of:
a) contacting a test compound with a cell into which a vector comprising a transcriptional regulatory region of a TBC1D7 gene and a reporter gene that is expressed under control of the transcriptional regulatory region has been introduced;
b) measuring expression or activity of said reporter gene; and
c) selecting a compound that reduces expression or activity level of said reporter gene, as compared to a level in absence of the test compound.

15. A method of screening for a candidate compound that inhibits a binding between a TBC1D7 polypeptide and a 14-3-3 zeta polypeptide, a RAB17 polypeptide, or a TSC1 polypeptide, said method comprising steps of:
(a) contacting TBC1D7 polypeptide or functional equivalent thereof with a 14-3-3 zeta, a RAB17, or a TSC1 polypeptide or functional equivalent thereof in presence of a test agent;
(b) detecting a binding between the polypeptides;
(c) comparing binding level detected in the step (b) with those detected in absence of the test agent; and
(d) selecting the test agent that reduces or inhibits binding level comparing with those detected in absence of the test agent in step (c).

16. The method of claim 15, wherein the functional equivalent of TBC1D7 comprises 14-3-3 zeta, RAB17, or TSC1-binding domain.

17. The method of claims 10, wherein the cancer is lung or esophageal cancer.

18. A double-stranded molecule comprising a sense strand and an antisense strand, wherein the sense strand comprises a nucleotide sequence corresponding to a target sequence consisting of SEQ ID NO: 18 or 19, and wherein the antisense strand comprises a nucleotide sequence which is complementary to said sense strand, wherein said sense strand and said antisense strand hybridize to each other to form said double-stranded molecule, and wherein said double-stranded molecule, when introduced into a cell expressing the TBC1D7 gene, inhibits expression of said gene.

19. The double-stranded molecule of claim 18, wherein the double-stranded molecule is an oligonucleotide of between about 19 and about 25 nucleotides in length.

20. The double-stranded molecule of claim 18, wherein said double-stranded molecule is a single nucleotide transcript comprising the sense strand and the antisense strand linked via a single-stranded nucleotide sequence.

21. The double-stranded molecule of claim 20, wherein said polynucleotide has a general formula 5’-[A]-[B]-[A′]-3′ wherein [A] is a nucleotide sequence comprising SEQ ID NO: 18 or 19; [B] is a nucleotide sequence consisting of about 3 to about 23 nucleotides; and [A′] is a nucleotide sequence complementary to [A].

22. The double-stranded molecule of claim 18, wherein a cell expressing the TBC1D7 gene is selected from the group of bladder cancer cell, gastric cancer cell, colon and rectum cancer cell, breast cancer cell, esophagus cancer cell, lung cancer cell, lymphoma cell, pancreatic cancer cell and testicular cancer cell.

23. A vector comprising each or both of a combination of polynucleotide comprising a sense strand nucleic acid and an antisense strand nucleic acid, wherein said sense strand nucleic acid comprises the nucleotide sequence of SEQ ID NOs: 18 or 19, and wherein the antisense strand comprises a nucleotide sequence which is complementary to said sense strand, wherein transcripts of said sense strand and said antisense strand hybridize to each other to form said double-stranded molecule, and wherein said vector, when introduced into a cell expressing the TBC1D7 gene, inhibits expression of said gene.

24. The vector of claim 23, wherein the polynucleotide is an oligonucleotide of between about 19 and about 25 nucleotides in length.

25. The vector of claim 23, wherein said double-stranded molecule is a single nucleotide transcript comprising the sense strand and the antisense strand linked via a single-stranded nucleotide sequence.

26. The vector of claim 25, wherein said polynucleotide has a general formula 5′-[A]-[B]-[A′]-3′ wherein [A] is a nucleotide sequence comprising SEQ ID NO: 18 or 19; [B] is a nucleotide sequence consisting of about 3 to about 23 nucleotides; and [A′] is a nucleotide sequence complementary to [A].

27. A method of treating or preventing cancer in a subject comprising administering to said subject a pharmaceutically effective amount of a double-stranded molecule against a TBC1D7 or a vector comprising said double-stranded molecule that inhibits cell proliferation contacting with a cell expressing TBC1D7 gene, and a pharmaceutically acceptable carrier.

28. The method of claim 27, wherein the double-stranded molecule comprises a sense strand and an antisense strand, wherein the sense strand comprises a nucleotide sequence corresponding to a target sequence consisting of SEQ ID NO: 18 or 19, and wherein the antisense strand comprises a nucleotide sequence which is complementary to said sense strand, wherein said sense strand and said antisense strand hybridize to each other to form said double-stranded molecule, and wherein said double-stranded molecule, when introduced into a cell expressing the TBC1D7 gene, inhibits expression of said gene, and wherein the vector comprises each or both of a combination of polynucleotide comprising a sense strand nucleic acid and an antisense strand nucleic acid, wherein said sense strand nucleic acid comprises the nucleotide sequence of SEQ ID NOs: 18 or 19, and wherein the antisense strand comprises a nucleotide sequence which is complementary to said sense strand, wherein transcripts of said sense strand and said antisense strand hybridize to each other to form said double-stranded molecule, and wherein said vector, when introduced into a cell expressing the TBC1D7 gene, inhibits expression of said gene.

29. The method of claim 27, wherein the cancer is selected from lung cancer and esophageal cancer.

30. A composition for treating or preventing cancer, which comprises a pharmaceutically effective amount of a double-stranded molecule against a TBC1D7 or a vector comprising said double-stranded molecule that inhibits cell proliferation when in contact with a cell expressing TBC1D7 gene, and a pharmaceutically acceptable carrier.

31. The composition of claim 30, wherein the double-stranded molecule comprises a sense strand and an antisense strand, wherein the sense strand comprises a nucleotide sequence corresponding to a target sequence consisting of SEQ ID NO: 18 or 19, and wherein the antisense strand comprises a nucleotide sequence which is complementary to said sense strand, wherein said sense strand and said antisense strand hybridize to each other to form said double-stranded molecule, and wherein said double-stranded molecule, when introduced into a cell expressing the TBC1D7 gene, inhibits expression of said gene, and wherein the vector comprises each or both of a combination of polynucleotide comprising a sense strand nucleic acid and an antisense strand nucleic acid, wherein said sense strand nucleic acid comprises the nucleotide sequence of SEQ ID NOs: 18 or 19, and wherein the antisense strand comprises a nucleotide sequence which is complementary to said sense strand, wherein transcripts of said sense strand and said antisense strand hybridize to each other to form said double-stranded molecule, and wherein said vector, when introduced into a cell expressing the TBC1D7 gene, inhibits expression of said gene.

32. The composition of claim 30, wherein the cancer is selected from the group of lung and esophageal cancer.

33. A polypeptide selected from the group consisting of:
(a) a polypeptide comprising YWITRRFVNQLNTKYRDSLP (SEQ ID NO: 28), and
(b) a polypeptide having an amino acid sequence of a polypeptide functionally equivalent to the polypeptide consisting of YWITRRFVNQLNTKYRDSLP (SEQ ID NO: 28),
wherein the polypeptide lacks the biological function of a polypeptide consisting of the amino acid sequence of SEQ ID NO: 2.

34. A polynucleotide encoding the polypeptide of claim 33.

35. The polypeptide of the claim 33, wherein the biological function is cell proliferation activity or invasion activity.

36. The polypeptide of claim 33, wherein the polypeptide consists of 20 to 60 residues.

37. The polypeptide of claim 33, wherein the polypeptide is modified with a cell-membrane permeable substance.

38. The polypeptide of claim 37, which has the following general formula: [R]-[D]; wherein [R] represents the cell-membrane permeable substance; and [D] represents the amino acid sequence of a fragment sequence which comprises YWITRRFVNQLNTKYRDSLP (SEQ ID NO: 28); or the amino acid sequence of a polypeptide functionally equivalent to the polypeptide comprising said fragment sequence, wherein the polypeptide lacks the biological function of a polypeptide consisting of the amino acid sequence of SEQ ID NO: 2, wherein [R] and [D] can be linked directly or indirectly through a linker.

39. The polypeptide of claim 38, wherein the cell-membrane permeable substance is any one selected from the group consisting of:
SEQ ID NO: 43
poly-arginine/RRRRRRRRRRR/;
SEQ ID NO: 29
Tat/RKKRRQRRR/;
SEQ ID NO: 30
Penetratin/RQIKIWFQNRRMKWKK/;
SEQ ID NO: 31
Buforin II/TRSSRAGLQFPVGRVHRLLRK/;
SEQ ID NO: 32
Transportan/GWTLNSAGYLLGKINLKALAALAKKIL/;
SEQ ID NO: 33
MAP (model amphipathic peptide)/
KLALKLALKALKAALKLA/;
SEQ ID NO: 34
K-FGF/AAVALLPAVLLALLAP/;
SEQ ID NO: 35
Ku70/VPMLK/
SEQ ID NO: 36
Ku70/PMLKE/;
SEQ ID NO: 37
Prion/MANLGYWLLALFVTMWTDVGLCKKRPKP/;
SEQ ID NO: 38
pVEC/LLIILRRRIRKQAHAHSK/;
SEQ ID NO: 39
Pep-1/KETWWETWWTEWSQPKKKRKV/;
SEQ ID NO: 40
SynB1/RGGRLSYSRRRFSTSTGR/;
SEQ ID NO: 41
Pep-7/SDLWEMMMVSLACQY/;
and
SEQ ID NO: 42
HN-1/TSPLNIHNGQKL/.

40. The method of claim 11, wherein the cancer is lung or esophageal cancer.

41. The method of claim 14, wherein the cancer is lung or esophageal cancer.


SEQUENCE LISTING (45 sequences)

SEQ ID NO: 1 - DNA, 1147 bp, Homo sapiens
  CDS (94)..(975)
cggcggtcggtcagcgcacagggcacggcttcctctgcttctcccaccacttagtctcaacccgggtgtgtttgcactactagagaatgaaatatgactgaggactctcagagaaactttcgttcagtatattatgagaaagtggggtttcgtggagttgaagaaaagaaatcattagaaattctcctaaaagatgaccgtctggatactgagaaactttgtacttttagtcagaggttccctctcccgtccatgtaccgtgcattggtatggaaggtgcttctaggaatcttgcctccacaccacgagtcccatgccaaggtgatgatgtatcgtaaggagcagtacttggatgtccttcatgccctgaaagtcgttcgctttgttagtgatgccacacctcaggctgaagtctatctccgcatgtatcagctggagtctgggaagttacctcgaagtccctcttttccactggagccagatgatgaagtgtttcttgccatagctaaagccatggaggaaatggtggaagatagtgtcgactgttactggatcacccgacgctttgtgaaccaattaaataccaagtaccgggattccttgccccagttgccaaaagcgtttgaacaatacttgaatctggaagatggcagactgctgactcatctgaggatgtgttccgcggcgcccaaacttccttatgatctctggttcaagaggtgctttgcgggatgtttgcctgaatccagtttacagagggtttgggataaagttgtgagtggatcctgtaagatcctagtttttgtagctgtcgaaattttattaacctttaaaataaaagttatggcactgaacagtgcagagaagataacaaagtttctggaaaatattccccaggacagctcagacgcgatcgtgagcaaggccattgacttgtggcacaaacactgtgggaccccggtccattcaagctgaacgcacccgctggttgtggaccgtctgccaggcaccacagtgagcattgtgttcttggcatgtgatctgggaaactgattgaataatacacttttcttgctttggtgctcaaagtggtttttttcccccaataaaattatttaattgaaaaaaaaaaaaaaaaaaaaaaaaa

SEQ ID NO: 2 - protein, 293 aa, Homo sapiens
MTEDSQRNFRSVYYEKVGFRGVEEKKSLEILLKDDRLDTEKLCTFSQRFPLPSMYRALVWKVLLGILPPHHESHAKVMMYRKEQYLDVLHALKVVRFVSDATPQAEVYLRMYQLESGKLPRSPSFPLEPDDEVFLAIAKAMEEMVEDSVDCYWITRRFVNQLNTKYRDSLPQLPKAFEQYLNLEDGRLLTHLRMCSAAPKLPYDLWFKRCFAGCLPESSLQRVWDKVVSGSCKILVFVAVEILLTFKIKVMALNSAEKITKFLENIPQDSSDAIVSKAIDLWHKHCGTPVHSS

SEQ ID NO: 3 - DNA, 21 bp, Artificial Sequence
  a target sequence of siRNA
gaacagtgcagagaagatatt

SEQ ID NO: 4 - DNA, 21 bp, Artificial Sequence
  a target sequence of siRNA
gataaagttgtgagtggattt

SEQ ID NO: 5 - DNA, 23 bp, Artificial Sequence
  an artificial synthesized RT-PCR primer
ccctagtttttgtagctgtcgaa

SEQ ID NO: 6 - DNA, 22 bp, Artificial Sequence
  an artificial synthesized RT-PCR primer
gatcacatgccaagaacacaat

SEQ ID NO: 7 - DNA, 21 bp, Artificial Sequence
  an artificial synthesized RT-PCR primer
gaggtgatagcattgctttcg

SEQ ID NO: 8 - DNA, 21 bp, Artificial Sequence
  an artificial synthesized RT-PCR primer
caagtcagtgtacaggtaagc

SEQ ID NO: 9 - DNA, 19 bp, Artificial Sequence
  a target sequence of siRNA
gaagcagcacgacttcttc

SEQ ID NO: 10 - DNA, 19 bp, Artificial Sequence
  a target sequence of siRNA
gcgcgctttgtaggattcg

SEQ ID NO: 11 - DNA, 1980 bp, Homo sapiens
  CDS (645)..(1283)
aaaaaaaaaaaaaaactcagttgcctctggccagtgcagggctcagccagggatggcttctagctgacagtgggaggaattaattcatctgaccggaatattcttttctcttctgggctgttggtttttcaagtgcaacaaagattccatacagctccaaggaaggagccaagaaaaacattctgtgccaaagtgagatcctggaagtgaaaccccggaataaagctgaaaagcgggctccagttgggtgccaggaaatgcaggactggaatgtgacttgacttccggcagcgcgcaggtgctcccgggtcacctgctttgaggtccagcctcctgccctgcctcaggtgaccacatgaccactgtggactttgccctgaaaccttctgggaggagaagaggcctgaccttggcgctggggtccagtgggcattgctctggtccgaggctgctgctcttgacctctgctctgcggctgttttccattggagtagaggctcctcctgtcctgtcctgcctgtggagggaagcaaaccttcccctggaccagagagaggagaaagcggagacaggtagcaacgctgtggactggtgatgacaggctcttcagctccctgcaagtgaccgggcctggggaacagggcatggcacaggcacacaggaccccccagcccagggctgcccccagccagccccgtgtgttcaagctggttctcctgggaagtggctccgtgggtaagtccagcttggctcttcggtacgtgaagaacgacttcaagagtatcctgcctacggtgggctgtgcgttcttcacaaaggtggtggatgtgggtgccacctctctgaagcttgagatctgggacacagctggccaggagaagtaccacagcgtctgccacctctacttcaggggtgccaacgctgcgcttctggtgtacgacatcaccaggaaggattccttcctcaaggctcagcagtggctgaaggacctggaggaggagctgcacccaggagaagtcctggtgatgctggtgggcaacaagacggacctcagccaggagcgggaggtgaccttccaggaagggaaggagtttgccgacagccagaagttgctgttcatggaaacttcggccaaactgaaccaccaggtgtcggaggtgttcaatacagtggcccaagagctactgcagagaagcgacgaggagggccaggctctacggggggatgcagctgtggctctgaacaaggggcccgcgaggcaggccaaatgctgcgcccactaggtgcagccactcctgggggctgtggggaagacaccccctgcctgggccatggccagctctaggtggattctgattcactgtcaatgctgggttgctcccgagccctagatgttcctggaagttggccccctttatgaaaaccacttcccacagccagtgggaactgccagaggaagatctggcgtcacatggctcccaggaaagtgctgtgccctatccccactgataccatctgattccccgatgcctgtgcctgttccacctggacggtggccccctcagcctggcagcctctggacagagaggaaggaaggattggaaaagtccccgcagcacagcgacggtgggaagatgccttacgtctgatcttgatgggggcactggcctggagcctgggcccacctgcttctggggggttggggagcaggccagatggaggtggtggtgccaggaagaaatggagcgatgactgactgtggggtgggcccaggatttccacatcttggtgaagttgcccctgggaagggcagctgggggcagtggcgccagttcccttccatggtctcccggctggcaatgtggtgaagctgagtttctgtccaatgagcaggaagattctgagacatttcgcctgagatataagttgtactgcgtatgcagtttttcctccaaaaattaaattgcttttgacaatctg

SEQ ID NO: 12 - protein, 212 aa, Homo sapiens
MAQAHRTPQPRAAPSQPRVFKLVLLGSGSVGKSSLALRYVKNDFKSILPTVGCAFFTKVVDVGATSLKLEIWDTAGQEKYHSVCHLYFRGANAALLVYDITRKDSFLKAQQWLKDLEEELHPGEVLVMLVGNKTDLSQEREVTFQEGKEFADSQKLLFMETSAKLNHQVSEVFNTVAQELLQRSDEEGQALRGDAAVALNKGPARQAKCCAH

SEQ ID NO: 13 - DNA, 2834 bp, Homo sapiens
  CDS (85)..(822)
gcccactcccaccgccagctggaaccctggggactacgacgtccctcaaaccttgcttctaggagataaaaagaacatccagtcatggataaaaatgagctggttcagaaggccaaactggccgagcaggctgagcgatatgatgacatggcagcctgcatgaagtctgtaactgagcaaggagctgaattatccaatgaggagaggaatcttctctcagttgcttataaaaatgttgtaggagcccgtaggtcatcttggagggtcgtctcaagtattgaacaaaagacggaaggtgctgagaaaaaacagcagatggctcgagaatacagagagaaaattgagacggagctaagagatatctgcaatgatgtactgtctcttttggaaaagttcttgatccccaatgcttcacaagcagagagcaaagtcttctatttgaaaatgaaaggagattactaccgttacttggctgaggttgccgctggtgatgacaagaaagggattgtcgatcagtcacaacaagcataccaagaagcttttgaaatcagcaaaaaggaaatgcaaccaacacatcctatcagactgggtctggcccttaacttctctgtgttctattatgagattctgaactccccagagaaagcctgctctcttgcaaagacagcttttgatgaagccattgctgaacttgatacattaagtgaagagtcatacaaagacagcacgctaataatgcaattactgagagacaacttgacattgtggacatcggatacccaaggagacgaagctgaagcaggagaaggaggggaaaattaaccggccttccaacttttgtctgcctcattctaaaatttacacagtagaccatttgtcatccatgctgtcccacaaatagttttttgtttacgatttatgacaggtttatgttacttctatttgaatttctatatttcccatgtggtttttatgtttaatattaggggagtagagccagttaacatttagggagttatctgttttcatcttgaggtggccaatatggggatgtggaatttttatacaagttataagtgtttggcatagtacttttggtacattgtggcttcaaaagggccagtgtaaaactgcttccatgtctaagcaaagaaaactgcctacatactggtttgtcctggcggggaataaaagggatcattggttccagtcacaggtgtagtaattgtgggtactttaaggtttggagcacttacaaggctgtggtagaatcataccccatggataccacatattaaaccatgtatatctgtggaatactcaatgtgtacacctttgactacagctgcagaagtgttcctttagacaaagttgtgacccattttactctggataagggcagaaacggttcacattccattatttgtaaagttacctgctgttagctttcattatttttgctacactcattttatttgtatttaaatgttttaggcaacctaagaacaaatgtaaaagtaaagatgcaggaaaaatgaattgcttggtattcattacttcatgtatatcaagcacagcagtaaaacaaaaacccatgtatttaacttttttttaggatttttgcttttgtgattttttttttttttttttgatacttgcctaacatgcatgtgctgtaaaaatagttaacagggaaataacttgagatgatggctagctttgtttaatgtcttatgaaattttcatgaacaatccaagcataattgttaagaacacgtgtattaaattcatgtaagtggaataaaagttttatgaatggacttttcaactactttctctacagcttttcatgtaaattagtcttggttctgaaacttctctaaaggaaattgtacattttttgaaatttattccttattccctcttggcagctaatgggctcttaccaagtttaaacacaaaatttatcataacaaaaatactactaatataactactgtttccatgtcccatgatcccctctcttcctccccaccctgaaaaaaatgagttcctattttttctgggagagggggggattgattagaaaaaaatgtagtgtgttccatttaaaattttggcatatggcattttctaacttaggaagccacaatgttcttggcccatcatgacattgggtagcattaactgtaagttttgtgcttccaaatcactttttggtttttaagaatttcttgatactcttatagcctgccttcaattttgatcctttattctttctatttgtcaggtgcacaagattaccttcctgttttagccttctgtcttgtcaccaaccattcttacttggtggccatgtacttggaaaaaggccgcatgatctttctggctccactcagtgtctaaggcaccctgcttcctttgcttgcatcccacagactatttccctcatcctatttactgcagcaaatctctccttagttgatgagactgtgtttatctccctttaaaaccctacctatcctgaatggtctgtcattgtctgcctttaaaatccttcctctttcttcctcctctattctctaaataatgatggggctaagttatacccaaagctcactttacaaaatatttcctcagtactttgcagaaaacaccaaacaaaaatgccattttaaaaaaggtgtattttttcttttagaatgtaagctcctcaagagcagggacaatgttttctgtatgttctattgtgcctagtacactgtaaatgctcaataaatattgatgatgggaggcagtgagtcttgatgataagggtgagaaactgaaatccc

SEQ ID NO: 14 - protein, 245 aa, Homo sapiens
MDKNELVQKAKLAEQAERYDDMAACMKSVTEQGAELSNEERNLLSVAYKNVVGARRSSWRVVSSIEQKTEGAEKKQQMAREYREKIETELRDICNDVLSLLEKFLIPNASQAESKVFYLKMKGDYYRYLAEVAAGDDKKGIVDQSQQAYQEAFEISKKEMQPTHPIRLGLALNFSVFYYEILNSPEKACSLAKTAFDEAIAELDTLSEESYKDSTLIMQLLRDNLTLWTSDTQGDEAEAGEGGEN

SEQ ID NO: 15 - DNA, 22 bp, Artificial Sequence
  an artificial synthesized RT-PCR primer
cctagtttttgtagctgtcgaa

SEQ ID NO: 16 - DNA, 20 bp, Artificial Sequence
  an artificial synthesized RT-PCR primer
ctccacagccagatcagaca

SEQ ID NO: 17 - DNA, 20 bp, Artificial Sequence
  an artificial synthesized RT-PCR primer
gctgcctgttcaagaactcc

SEQ ID NO: 18 - RNA, 19 nt, Artificial Sequence
  a target sequence of siRNA
gaacagugcagagaagaua

SEQ ID NO: 19 - RNA, 19 nt, Artificial Sequence
  a target sequence of siRNA
gauaaaguugugaguggau

SEQ ID NO: 20 - RNA, 19 nt, Artificial Sequence
  a target sequence of siRNA
cgacacggcugauaacuga

SEQ ID NO: 21 - RNA, 19 nt, Artificial Sequence
  a target sequence of siRNA
cguacgcggaauacuucga

SEQ ID NO: 22 - RNA, 19 nt, Artificial Sequence
  a target sequence of siRNA
gaagcagcacgacuucuuc

SEQ ID NO: 23 - protein, 34 aa, Artificial Sequence
  a dominant-negative peptide
RRRRRRRRRRRGGGYQLESGKLPRSPSFPLEPDD

SEQ ID NO: 24 - protein, 34 aa, Artificial
  a dominant-negative peptide
RRRRRRRRRRRGGGEVFLAIAKAMEEMVEDSVDC

SEQ ID NO: 25 - protein, 34 aa, Artificial
  a dominant-negative peptide
RRRRRRRRRRRGGGYWITRRFVNQLNTKYRDSLP

SEQ ID NO: 26 - protein, 20 aa, Artificial
  a dominant-negative peptide
YQLESGKLPRSPSFPLEPDD

SEQ ID NO: 27 - protein, 20 aa, Artificial
  a dominant-negative peptide
EVFLAIAKAMEEMVEDSVDC

SEQ ID NO: 28 - protein, 20 aa, Artificial
  a dominant-negative peptide
YWITRRFVNQLNTKYRDSLP

SEQ ID NO: 29 - protein, 9 aa, Artificial
  a cell-membrane permeable substance
RKKRRQRRR

SEQ ID NO: 30 - protein, 16 aa, Artificial
  a cell-membrane permeable substance
RQIKIWFQNRRMKWKK

SEQ ID NO: 31 - protein, 21 aa, Artificial
  a cell-membrane permeable substance
TRSSRAGLQFPVGRVHRLLRK

SEQ ID NO: 32 - protein, 27 aa, Artificial
  a cell-membrane permeable substance
GWTLNSAGYLLGKINLKALAALAKKIL

SEQ ID NO: 33 - protein, 18 aa, Artificial
  a cell-membrane permeable substance
KLALKLALKALKAALKLA

SEQ ID NO: 34 - protein, 16 aa, Artificial
  a cell-membrane permeable substance
AAVALLPAVLLALLAP

SEQ ID NO: 35 - protein, 5 aa, Artificial
  a cell-membrane permeable substance
VPMLK

SEQ ID NO: 36 - protein, 5 aa, Artificial
  a cell-membrane permeable substance
PMLKE

SEQ ID NO: 37 - protein, 28 aa, Artificial
  a cell-membrane permeable substance
MANLGYWLLALFVTMWTDVGLCKKRPKP

SEQ ID NO: 38 - protein, 18 aa, Artificial
  a cell-membrane permeable substance
LLIILRRRIRKQAHAHSK

SEQ ID NO: 39 - protein, 21 aa, Artificial
  a cell-membrane permeable substance
KETWWETWWTEWSQPKKKRKV

SEQ ID NO: 40 - protein, 18 aa, Artificial
  a cell-membrane permeable substance
RGGRLSYSRRRFSTSTGR

SEQ ID NO: 41 - protein, 15 aa, Artificial
  a cell-membrane permeable substance
SDLWEMMMVSLACQY

SEQ ID NO: 42 - protein, 12 aa, Artificial
  a cell-membrane permeable substance
TSPLNIHNGQKL

SEQ ID NO: 43 - protein, 11 aa, Artificial
  a cell-membrane permeable substance
RRRRRRRRRRR

SEQ ID NO: 44 - DNA, 8626 bp, Homo sapiens
  CDS (235)..(3729)
acgacgggggaggtgctgtacgtccaagatggcggcgccctgtaggctggagggactgtgaggtaaacagctgagggggaggagacggtggtgaccatgaaagacaccaggttgacagcactggaaactgaagtaccagttgtcgctagaacagtttggtagtggccccaatgaagaaccttcagaacctgtagcacacgtcctggagccagcacagcgccttcgagcgagagaatggcccaacaagcaaatgtcggggagcttcttgccatgctggactcccccatgctgggtgtgcgggacgacgtgacagctgtctttaaagagaacctcaattctgaccgtggccctatgcttgtaaacaccttggtggattattacctggaaaccagctctcagccggcattgcacatcctgaccaccttgcaagagccacatgacaagcacctcttggacaggattaacgaatatgtgggcaaagccgccactcgtttatccatcctctcgttactgggtcatgtcataagactgcagccatcttggaagcataagctctctcaagcacctcttttgccttctttactaaaatgtctcaagatggacactgacgtcgttgtcctcacaacaggcgtcttggtgttgataaccatgctaccaatgattccacagtctgggaaacagcatcttcttgatttctttgacatttttggccgtctgtcatcatggtgcctgaagaaaccaggccacgtggcggaagtctatctcgtccatctccatgccagtgtgtacgcactctttcatcgcctttatggaatgtacccttgcaacttcgtctcctttttgcgttctcattacagtatgaaagaaaacctggagacttttgaagaagtggtcaagccaatgatggagcatgtgcgaattcatccggaattagtgactggatccaaggaccatgaactggaccctcgaaggtggaagagattagaaactcatgatgttgtgatcgagtgtgccaaaatctctctggatcccacagaagcctcatatgaagatggctattctgtgtctcaccaaatctcagcccgctttcctcatcgttcagccgatgtcaccaccagcccttatgctgacacacagaatagctatgggtgtgctacttctaccccttactccacgtctcggctgatgttgttaaatatgccagggcagctacctcagactctgagttccccatcgacacggctgataactgaaccaccacaagctactctttggagcccatctatggtttgtggtatgaccactcctccaacttctcctggaaatgtcccacctgatctgtcacacccttacagtaaagtctttggtacaactgcaggtggaaaaggaactcctctgggaaccccagcaacctctcctcctccagccccactctgtcattcggatgactacgtgcacatttcactcccccaggccacagtcacaccccccaggaaggaagagagaatggattctgcaagaccatgtctacacagacaacaccatcttctgaatgacagaggatcagaagagccacctggcagcaaaggttctgtcactctaagtgatcttccagggtttttaggtgatctggcctctgaagaagatagtattgaaaaagataaagaagaagctgcaatatctagagaactttctgagatcaccacagcagaggcagagcctgtggttcctcgaggaggctttgactctcccttttaccgagacagtctcccaggttctcagcggaagacccactcggcagcctccagttctcagggcgccagcgtgaaccctgagcctttacactcctccctggacaagcttgggcctgacacaccaaagcaagcctttactcccatagacctgccctgcggcagtgctgatgaaagccctgcgggagacagggaatgccagacttctttggagaccagtatcttcactcccagtccttgtaaaattccacctccgacgagagtgggctttggaagcgggcagcctcccccgtatgatcatctttttgaggtggcattgccaaagacagcccatcattttgtcatcaggaagactgaggagctgttaaagaaagcaaaaggaaacacagaggaagatggtgtgccctctacctccccaatggaagtgctggacagactgatacagcagggagcagacgcgcacagcaaggagctgaacaagttgcctttacccagcaagtctgtcgactggacccactttggaggctctcctccttcagatgagatccgcaccctccgagaccagttgcttttactgcacaaccagttactctatgagcgttttaagaggcagcagcatgccctccggaacaggcggctcctccgcaaggtgatcaaagcagcagctctggaggaacataatgctgccatgaaagatcagttgaagttacaagagaaggacatccagatgtggaaggttagtctgcagaaagaacaagctagatacaatcagctccaggagcagcgtgacactatggtaaccaagctccacagccagatcagacagctgcagcatgaccgagaggaattctacaaccagagccaggaattacagacgaagctggaggactgcaggaacatgattgcggagctgcggatagaactgaagaaggccaacaacaaggtgtgtcacactgagctgctgctcagtcaggtttcccaaaagctctcaaacagtgagtcggtccagcagcagatggagttcttgaacaggcagctgttggttcttggggaggtcaacgagctctatttggaacaactgcagaacaagcactcagataccacaaaggaagtagaaatgatgaaagccgcctatcggaaagagctagaaaaaaacagaagccatgttctccagcagactcagaggcttgatacctcccaaaaacggattttggaactggaatctcacctggccaagaaagaccaccttcttttggaacagaagaaatatctagaggatgtcaaactccaggcaagaggacagctgcaggccgcagagagcaggtatgaggctcagaaaaggataacccaggtgtttgaattggagatcttagatttatatggcaggttggagaaagatggcctcctgaaaaaacttgaagaagaaaaagcagaagcagctgaagcagcagaagaaaggcttgactgttgtaatgacgggtgctcagattccatggtagggcacaatgaagaggcatctggccacaacggtgagaccaagacccccaggcccagcagcgcccggggcagtagtggaagcagaggtggtggaggcagcagcagcagcagcagcgagctttctaccccagagaaacccccacaccagagggcaggcccattcagcagtcggtgggagacgactatgggagaagcgtctgccagcatccccaccactgtgggctcacttcccagttcaaaaagcttcctgggtatgaaggctcgagagttatttcgtaataagagcgagagccagtgtgatgaggacggcatgaccagtagcctttctgagagcctaaagacagaactgggcaaagacttgggtgtggaagccaagattcccctgaacctagatggccctcacccgtctcccccgaccccggacagtgttggacagctacatatcatggactacaatgagactcatcatgaacacagctaaggaatgatggtcaatcagtgttaacttgcatattgttggcacagaacaggaggtgtgaatgcacgtttcaaagctttcctgtttccagggtctgagtgcaagttcatgtgtggaaatgggacggaggtcctttggacagctgactgaatgcagaacggtttttggatctggcattgaaatgcctcttgaccttcccctccacccgccctaaccccctctcatttacctcgcagtgtgttctaatccaagggccagttggtgttcctcagtagctttactttcttcctttcccccccaaatggttgcgtcctttgaacctgtgcaatatgaggccaaatttaatctttgagtctaacacaccactttctgctttcccgaagttcagataactgggttggctctcaattagaccaggtagtttgttgcattgcaggtaagtctggttttgtcccttccaggaggacatagcctgcaaagctggttgtctttacatgaaagcgtttacatgagactttccgactgcttttttgattctgaagttcagcatctaaagcagcaggtctagaagaacaacggtttattcatacttgcattcttttggcagttctgataagcttcctagaaagttctgtgtaaacagaagcctgtttcagaaatctggagctggcactgtggagaccacacaccctttgggaaagctcttgtctcttcttcccccactacctcttatttatttggtgtttgcttgaatgctggtactattgtgaccacaggctggtgtgtaggtggtaaaacctgttctccataggagggaaggagcagtcactgggagaggttacccgagaagcacttgagcatgaggaactgcacctttaggccatctcagcttgctgggccttttgttaaacccttctgtctactggcctccctttgtgtgcatacgcctcttgttcatgtcagcttatatgtgacactgcagcagaaaggctctgaaggtccaaagagtttctgcaaagtgtatgtgaccatcatttcccaggccattagggttgcctcactgtagcaggttctaggctaccagaagaggggcagctttttcataccaattccaactttcaggggctgactctccagggagctgatgtcatcacactctccatgttagtaatggcagagcagtctaaacagagtccgggagaatgctggcaaaggctggctgtgtatacccactaggctgccccacgtgctcccgagagatgacactagtcagaaaattggcagtggcagagaatccaaactcaacaagtgctcctgaaagaaacgctagaagcctaagaactgtggtctggtgttccagctgaggcagggggatttggtaggaaggagccagtgaacttggctttcctgtttctatctttcattaaaaagaatagaaggattcagtcataaagaggtaaaaaactgtcacggtacgaaatcttagtgcccacggaggcctcgagcagagagaatgaaagtcttttttttttttttttttttttagcatggcaataaatattctagcatccctaactaaaggggactagacagttagagactctgtcaccctagctataccagcagaaaacctgttcaggcaggctttctgggtgtgactgattcccagcctgtggcagggcgtggtcccaactactcagcctagcacaggctggcagttggtactgaattgtcagatgtggagtattagtgacaccacacatttaattcagctttgtccaaaggaaagcttaaaacccaatacagtctagtttcctggttccgttttagaaaaggaaaacgtgaacaaacttagaaagggaaggaaatcccatcagtgaatcctgaaactggttttaagtgctttccttctcctcatgcccaagagatctgtgccatagaacaagataccaggcacttaaagccttttcctgaattggaaaggaaaagaggcccaagtgcaaaagaaaaaacattttagaaacggacagcttataaaaataaagggaagaaaggaggcagcatggagagaggcctgtgctagaagctccatggacgtgtctgcacagggtcctcagctcatccatgcggcctgggtgtccttttactcagctttataacaaatgtggctccaagctcaggtgcctttgagttctaggaggctgtgggttttattcaactacggttgggagaatgagacctggagtcatgttgaaggtgcccaacctaaaaatgtaggctttcatgttgcaaagaactccagagtcagtagttaggtttggtttggttttggacatgataaacctgccaagagtcaacaggtcacttgatcatgctgcagtgggtagttctaaggatggaaaggtgacagtattactctcgagaggcaattcagtcctgggcaaaggtattagtacaataagcgttaagggcagagtctaccttgaaaccaattaagcagcttggtattcataaatattgggattggatggcctccatccagaaatcactatgggtgagcatacctgtctcagctgtttggccaatgtgcataacctactcggatccccacctgacactaaccagagtcagcacaggccccgaggagcccgaagtctgctgctgtgcagcatggaattcctttaaaaaggtgcactacagttttagcggggagggggataggaagacgcagagcaaatgagctccggagtccctgcaggtgaataaacacacagatctgcatctgatagaactttgatggattttcaaaaagccgttgacaaggctctgctatacagtctataaaaattgttattatgggattggaagaaacacgtggtcatgaatagaaaaaaaacaaacccaaaggtaggaaggtcaaggtcatttcttagatggagaagttgtgaaagatgtccttggagatgagttttaggaccagcattactaaggcaggtgggcagacagtgacctctctaggtgtgtccacagagtttttcaggagagaaaactgcctgacctttgggactaagctgcggaatcttcttactaagcttgaagagtggagaggcgagaggtgagctactttgtgagccaaagcttatgtgacatggttggggaaacagtccaaactgttctgagaaggtgaactgttacgacccaggacaattagaaaaattcacccaccatgccgcacattactgggtaaaagcagggcagcagggaacaaaactccagactcttgggccgtccccatttgcaacagcacacatagtttctggtatatttgttgggaaagataaaactctagcagttgttgaggggaggatgtataaaatggtcatggggatgaaaggatctctgagaccacagaggctcagactcactgttaagaatagaaaactgggtatgcgtttcatgtagccagcagaactgaagtgtgctgtgacaagccaatgtgaatttctaccaaatagtagagcataccacttgaagaaggaaagaaccgaagagcaaacaaaagttctgcgtaatgagactcaccttttctcgctgaaagcactaagaggtgggaggaggcctgcacaggctggaggagggtttgggcagagcgaagacccggccaggaccttggtgagatggggtgccgcccacctcctgcggatactcttggagagttgttcccccagggggctctgccccacctggagaaggaagctgcctggtgtggagtgactcaaatcagtatacctatctgctgcaccttcactctccagggtacatgctttaaaaccgacccgcaacaagtattggaaaaatgtatccagtctgaagatgtttgtgtatctgtttacatccagagttctgtgacacatgccccccagattgctgcaaagatcccaaggcattgattgcacttgattaagcttttgtctgtaggtgaaagaacaagtttaggtcgaggactggcccctaggctgctgctgtgacccttgtcccatgtggcttgtttgcctgtccgggactcttcgatgtgcccaggggagcgtgttcctgtctcttccatgccgtcctgcagtccttatctgctcgcctgagggaagagtagctgtagctacaagggaagcctgcctggaagagccgagcacctgtgcccatggcttctggtcatgaaacgagttaatgatggcagaggagcttcctccccacttcgcagcgccacattatccatcctctgagataagtaggctggtttaaccattggaatggacctttcagtggaaaccctgagagtctgagaacccccagaccaacccttccctccctttccccacctcttacagtgtttggacaggagggtatggtgctgctctgtgtagcaagtactttggcttatgaaagaggcagccacgcattttgcactaggaagaatcagtaatcacttttcagaagacttctatggaccacaaatatattacggaggaacagattttgctaagacataatctagttttataactcaatcatgaatgaaccatgtgtggcaaacttgcagtttaaaggggtcccatcagtgaaagaaactgattttttttaacggactgcttttagttaaattgaagaaagtcagctcttgtcaaaaggtctaaactttcccgcctcaatcctaaaagcatgtcaacaatccacatcagatgccataaatatgaactgcaggataaaatggtacaatcttagtgaatgggaattggaatcaaaagagtttgctgtccttcttagaatgttctaaaatgtcaaggcagttgcttgtgtttaactgtgaacaaataaaaatttattgttttgcactacaaaaaaaaaa

SEQ ID NO: 45 - protein, 1164 aa, Homo sapiens
MAQQANVGELLAMLDSPMLGVRDDVTAVFKENLNSDRGPMLVNTLVDYYLETSSQPALHILTTLQEPHDKHLLDRINEYVGKAATRLSILSLLGHVIRLQPSWKHKLSQAPLLPSLLKCLKMDTDVVVLTTGVLVLITMLPMIPQSGKQHLLDFFDIFGRLSSWCLKKPGHVAEVYLVHLHASVYALFHRLYGMYPCNFVSFLRSHYSMKENLETFEEVVKPMMEHVRIHPELVTGSKDHELDPRRWKRLETHDVVIECAKISLDPTEASYEDGYSVSHQISARFPHRSADVTTSPYADTQNSYGCATSTPYSTSRLMLLNMPGQLPQTLSSPSTRLITEPPQATLWSPSMVCGMTTPPTSPGNVPPDLSHPYSKVFGTTAGGKGTPLGTPATSPPPAPLCHSDDYVHISLPQATVTPPRKEERMDSARPCLHRQHHLLNDRGSEEPPGSKGSVTLSDLPGFLGDLASEEDSIEKDKEEAAISRELSEITTAEAEPVVPRGGFDSPFYRDSLPGSQRKTHSAASSSQGASVNPEPLHSSLDKLGPDTPKQAFTPIDLPCGSADESPAGDRECQTSLETSIFTPSPCKIPPPTRVGFGSGQPPPYDHLFEVALPKTAHHFVIRKTEELLKKAKGNTEEDGVPSTSPMEVLDRLIQQGADAHSKELNKLPLPSKSVDWTHFGGSPPSDEIRTLRDQLLLLHNQLLYERFKRQQHALRNRRLLRKVIKAAALEEHNAAMKDQLKLQEKDIQMWKVSLQKEQARYNQLQEQRDTMVTKLHSQIRQLQHDREEFYNQSQELQTKLEDCRNMIAELRIELKKANNKVCHTELLLSQVSQKLSNSESVQQQMEFLNRQLLVLGEVNELYLEQLQNKHSDTTKEVEMMKAAYRKELEKNRSHVLQQTQRLDTSQKRILELESHLAKKDHLLLEQKKYLEDVKLQARGQLQAAESRYEAQKRITQVFELEILDLYGRLEKDGLLKKLEEEKAEAAEAAEERLDCCNDGCSDSMVGHNEEASGHNGETKTPRPSSARGSSGSRGGGGSSSSSSELSTPEKPPHQRAGPFSSRWETTMGEASASIPTTVGSLPSSKSFLGMKARELFRNKSESQCDEDGMTSSLSESLKTELGKDLGVEAKIPLNLDGPHPSPPTPDSVGQLHIMDYNETHHEHS